(19) United States
(12) Patent Application Publication
(10) Pub. No.: US 2024/0141325 A1
(43) Pub. Date: May 2, 2024

(54) GENERATION OF NOVEL CRISPR GENOME EDITING AGENTS USING COMBINATORIAL CHEMISTRY

(71) Applicant: Duke University, Durham, NC (US)

(72) Inventors: Bruce SULLENGER, Durham, NC (US); Korie BUSH, Durham, NC (US); Telmo LLANGA, Durham, NC (US)

(21) Appl. No.: 18/550,928

(22) PCT Filed: Mar. 15, 2022
(86) PCT No.: PCT/US2022/020416

§ 371 Date: Sep. 15, 2023

Related U.S. Application Data
(60) Provisional application No. 63/161,222, filed on Mar. 15, 2021.

(51) Int. Cl. C12N 15/10; C12N 9/12; C12N 9/22; C12N 15/113

(52) U.S. Cl. CPC C12N 15/1048; C12N 9/1264; C12N 9/22; C12N 15/113; C12N 2310/141; C12N 2310/20; C12N 2320/13

(57) ABSTRACT

Methods of generating novel guide nucleic acids comprising a template-conserved target complementary region to a template and template-randomized region, novel guide nucleic acids generated by the methods, mixtures and complexes comprising the novel guide nucleic acids are disclosed.

### CROSS-REFERENCE TO RELATED APPLICATIONS

This patent application claims the benefit of priority of U.S. Provisional Patent Application No. 63/161,222, filed Mar. 15, 2021, which is incorporated herein by reference in its entirety.

This application includes an electronically filed Sequence Listing submitted in .txt format. The Sequence Listing is entitled “155554.00638_ST25.txt” was created on Apr. 28, 2022, is 149,600 bytes in size and is hereby incorporated by reference in its entirety.

### BACKGROUND OF THE INVENTION

Current gene editing approaches to genetic therapy are based upon targeted DNA endonucleases such as CRISPR/Cas9-based RNA-guided DNA endonucleases (RGENs) and other Cas based technologies that utilize Cas/gRNA complexes as a means to target specific nucleotide sequences for expression, repression, and template-based editing. Critical to the use of Cas-based technologies is the binding interaction between the Cas protein and the guide RNA (gRNA or sgRNA). As a result, a need exists for novel guide nucleic acids for optimizing the guide nucleic acids and their interaction with the Cas proteins.

### BRIEF SUMMARY OF THE INVENTION

In one aspect of the current disclosure, methods for generating guide nucleic acids that bind a Cas protein are provided. In some embodiments, the methods comprise: (a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′portion and an invariant 3′ end, (b) partitioning candidate guide nucleic acids having an increased binding affinity to the Cas protein from candidate guide nucleic acids having a reduced binding affinity to the Cas protein; and (c) amplifying the candidate guide nucleic acids having the increased binding affinity to the Cas protein to generate a candidate mixture enriched for candidate guide nucleic acids having binding affinity for the Cas protein. In some embodiments, the candidate mixture is enriched for candidate guide nucleic acids having binding affinity for the Cas protein. In some embodiments, the candidate mixture enriched for candidate guide nucleic acids having binding affinity for the Cas protein is provided by: (i) contacting the Cas protein with the candidate guide nucleic acids and the target nucleic acid, (ii) partitioning candidate guide nucleic acids of step (i) having an increased binding affinity to the Cas protein from candidate guide nucleic acids having a reduced binding affinity to the Cas protein; and (iii) amplifying the candidate guide nucleic acids of step (i) having the increased binding affinity to the Cas protein from step (ii) to generate the candidate mixture enriched for candidate guide nucleic acids having binding affinity for the Cas protein. In some embodiments, the Cas protein is a Cas9 endonuclease. In some embodiments, the cleaved double-stranded target nucleic acid further comprises a second label. In some embodiments, the Cas9 endonuclease is Streptococcus pyogenes Cas9 endonuclease or functional variant thereof. In some embodiments, the Cas9 endonuclease is Staphylococcus aureus Cas9 endonuclease or functional variant thereof.

In another aspect of the current disclosure, methods for generating guide nucleic acids that allow cleavage of a double-stranded nucleic acid target when in complex with a Cas protein are provided. In some embodiments, the methods comprise: (a) contacting a Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′ portion and an invariant 3′ end, thereby forming one or more Cas protein-candidate guide nucleic acid complexes; (b) partitioning candidate guide nucleic acids having an increased Cas complex cleavage activity by selecting the Cas protein-candidate guide nucleic acid complexes having a free single-stranded DNA 3′ end from candidate guide nucleic acids having a reduced Cas complex cleavage activity; and (c) amplifying the candidate guide nucleic acids having the increased Cas complex cleavage activity to generate a candidate mixture enriched for candidate guide nucleic acids having Cas complex cleavage activity. In some embodiments, the Cas protein is further contacted with a polymerase and a labeled nucleotide and the partitioning step comprises labeling the free PAM-distal non-target strand with the labeled nucleotide. In some embodiments, the polymerase is a terminal deoxynucleotidyl transferase (TdT) and/or the labeled nucleotide is biotin-16-aminoallyl-2′-dATP. In some embodiments, the candidate mixture is enriched for candidate guide nucleic acids having binding affinity for the Cas protein. In some embodiments, the candidate mixture enriched for candidate guide nucleic acids having binding affinity for the Cas protein is provided by: (i) contacting the Cas protein with the candidate guide nucleic acids and the target nucleic acid, (ii) partitioning candidate guide nucleic acids of step (i) having an increased binding affinity to the Cas protein from candidate guide nucleic acids having a reduced binding affinity to the Cas protein; and (iii) amplifying the candidate guide nucleic acids of step (i) having the increased binding affinity to the Cas protein from step (ii) to generate the candidate mixture enriched for candidate guide nucleic acids having binding affinity for the Cas protein. In some embodiments, the Cas protein is a Cas9 endonuclease. In some embodiments, the cleaved double-stranded target nucleic acid further comprises a second label. In some embodiments, the Cas9 endonuclease is Streptococcus pyogenes Cas9 endonuclease or functional variant thereof. In some embodiments, the Cas9 endonuclease is Staphylococcus aureus Cas9 endonuclease or functional variant thereof.

In another aspect of the current disclosure, methods for generating a guide nucleic acid having miRNA activity are provided. In some embodiments, the methods comprise: (a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′ portion and an invariant 3′ end, (b) partitioning candidate guide nucleic acids having an increased binding affinity to the Cas protein from candidate guide nucleic acids having a reduced binding affinity to the Cas protein; and (c) amplifying the candidate guide nucleic acids having the increased binding affinity to the Cas protein to generate a candidate mixture enriched for candidate guide nucleic acids having binding affinity for the Cas protein and identifying an amplified candidate guide nucleic acid having the miRNA domain, and optionally isolating or purifying the amplified candidate guide nucleic acid having the miRNA domain. In some embodiments, the methods comprise: (a) contacting a Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′ portion and an invariant 3′ end, thereby forming one or more Cas protein-candidate guide nucleic acid complexes; (b) partitioning candidate guide nucleic acids having an increased Cas complex cleavage activity by selecting the Cas protein-candidate guide nucleic acid complexes having a free single-stranded DNA 3′ end from candidate guide nucleic acids having a reduced Cas complex cleavage activity; and (c) amplifying the candidate guide nucleic acids having the increased Cas complex cleavage activity to generate a candidate mixture enriched for candidate guide nucleic acids having Cas complex cleavage activity. In some embodiments, the candidate guide nucleic acids comprise a template-conserved miRNA domain. In some embodiments, the methods further comprise identifying an amplified candidate guide nucleic acid having a miRNA binding domain, and optionally isolating or purifying the amplified candidate guide nucleic acid having the miRNA binding domain. In some embodiments, the candidate guide nucleic acids comprise a template-conserved miRNA binding domain. In some embodiments, the method comprises identifying an amplified candidate guide nucleic acid having Cas complex cleavage activity greater than the template, and optionally isolating or purifying the amplified candidate guide nucleic acid. In some embodiments, the increased Cas complex cleavage activity is cell type specific.

In another aspect of the current disclosure, guide nucleic acids are provided. In some embodiments, the guide nucleic acids comprise a template-conserved target complementary region and a template-randomized scaffold, wherein the template-conserved target complementary region is configured to hybridize to a double-stranded nucleic acid target proximate to a protospacer adjacent motif (PAM) and wherein the template-randomized region has binding affinity for a Cas protein, wherein the guide nucleic acid comprises any one of the RNAs according to Table 1, Table 2, or Table 3. In some embodiments, the guide nucleic acid comprises a functional site, wherein the functional site is optionally a miRNA domain or a miRNA binding domain. In some embodiments, a complex formed by the guide nucleic acid and the Cas protein has Cas complex cleavage activity. In some embodiments, a complex formed by the guide nucleic acid and the Cas protein has Cas complex cleavage activity greater than the template gRNA-Cas complex in the presence of miRNA. In some embodiments, a complex formed by the guide nucleic acid and the Cas protein has cell-specific, increased Cas complex cleavage activity than the template gRNA-Cas complex. In some embodiments, the Cas protein the guide nucleic acid binds to is a Cas9 endonuclease, and optionally wherein the Cas9 endonuclease is Streptococcus pyogenes Cas9 endonuclease or Staphylococcus aureus Cas9 endonuclease or functional variants thereof.

In another aspect of the current disclosure, mixtures are provided. In some embodiments, the mixtures are comprised of more than one candidate guide nucleic acid, the candidate guide nucleic acids having a common template-conserved target complementary region and each candidate guide nucleic acid having a distinct template-randomized scaffold, wherein the template-conserved target complementary region is configured to hybridize to a double-stranded DNA proximate to a protospacer adjacent motif (PAM) and wherein the template-randomized scaffold has binding affinity for a Cas protein. In some embodiments, the mixtures further comprise a polymerase and a labeled nucleotide. In some embodiments, the polymerase is a terminal deoxynucleotidyl transferase (TdT). In some embodiments, the labeled nucleotide is biotin-16-aminoallyl-2′-dATP. In some embodiments, the mixture is enriched for candidate guide nucleic acids having binding affinity for a Cas protein and/or Cas complex cleavage activity. In some embodiments, the mixture was made by the methods provided herein. In some embodiments, at least one of the candidate guide nucleic acids is selected from the guide nucleic acids comprising a template-conserved target complementary region and a template-randomized scaffold, wherein the template-conserved target complementary region is configured to hybridize to a double-stranded nucleic acid target proximate to a protospacer adjacent motif (PAM) and wherein the template-randomized region has binding affinity for a Cas protein, wherein the guide nucleic acid comprises any one of the RNAs according to Table 1, Table 2, or Table 3.

In another aspect of the current disclosure, Cas complexes are provided. In some embodiments, the Cas complexes comprise: (a) a Cas protein, (b) a candidate guide nucleic acid, the candidate guide nucleic acid comprising a template-conserved target complementary region and a template-randomized scaffold having binding affinity for the Cas protein; and (c) a cleaved target nucleic acid, the cleaved target nucleic acid comprising a free single-stranded labeled 3′ end. In some embodiments, the Cas protein is a Cas9 endonuclease. In some embodiments, the Cas9 endonuclease is Streptococcus pyogenes Cas9 endonuclease, Staphylococcus aureus Cas9 endonuclease or a functional variant thereof. In some embodiments, the free single-stranded labeled 3′ end of the target nucleic acid is biotinylated. In some embodiments, the cleaved target nucleic acid further comprises a second label. In some embodiments, the candidate guide nucleic comprises one or more candidate guide nucleic acids comprising a template-conserved target complementary region and a template-randomized scaffold, wherein the template-conserved target complementary region is configured to hybridize to a double-stranded nucleic acid target proximate to a protospacer adjacent motif (PAM) and wherein the template-randomized region has binding affinity for a Cas protein, wherein the guide nucleic acid comprises any one of the RNAs according to Table 1, Table 2, or Table 3 or the candidate mixture comprising more than one candidate guide nucleic acid, the candidate guide nucleic acids having a common template-conserved target complementary region and each candidate guide nucleic acid having a distinct template-randomized scaffold, wherein the template-conserved target complementary region is configured to hybridize to a double-stranded DNA proximate to a protospacer adjacent motif (PAM) and wherein the template-randomized scaffold has binding affinity for a Cas protein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates a Cas complex. sgRNA (SEQ ID NO: 579); Target strand 1 (SEQ ID NO: 566); Target strand 2 (SEQ ID NO: 567).

FIG. 2 illustrates a guide nucleic acid.

FIG. 3 illustrates candidate guide nucleic acid generation (SEQ ID NO: 568-571 from top to bottom).

FIG. 4 illustrates an exemplary system for partitioning candidate guide nucleic acids.

FIG. 5 illustrates an exemplary method for partitioning candidate guide nucleic acids.

FIG. 6 illustrates a Cas complex having a cleaved double-stranded DNA forming a single-stranded DNA 3′ end which can form a labeled PAM-distal non-target strand.

FIG. 7 illustrates a Cas9 cleavage assay after successive rounds of selection.

FIG. 8 illustrates cleavage activity of candidate guide nucleic acids. Consensus sequence: SEQ ID NO: 580 and 572; Original Guide RNA: SEQ ID NO: 581; Colony 254 Plate: SEQ ID NO: 573; Colony 264 Plate: SEQ ID NO: 574; Colony 258 Plate: SEQ ID NO: 575, Colony 243 Plate: SEQ ID NO: 576.

FIG. 9 illustrates cleavage activity of candidate guide nucleic acids 1-20. Order of guides is presented in accordance with their abundance from high to low.

FIG. 10 illustrates cleavage activity of candidate guide nucleic acids 21-34. Order of guides is presented in accordance with their abundance from high to low.

FIG. 11 illustrates cleavage activity of candidate guide nucleic acids 35-48. Order of guides is presented in accordance with their abundance from high to low.

FIG. 12 illustrates cleavage activity of candidate guide nucleic acids 49-60. Order of guides is presented in accordance with their abundance from high to low.

FIG. 13 illustrates sequence diversity of functional guide nucleic acids, with different colors representing different nucleotides (SEQ ID NO: 582).

FIG. 14 illustrates that variants derived from the gRNA functional selection are capable of cleaving target DNA.

FIG. 15 illustrates sequence diversity of functional guide nucleic acids identified with labeled PAM-distal non-target strand enhanced selection, with different colors representing different nucleotides (SEQ ID NO: 582).

FIG. 16 illustrates sequence diversity of functional guide nucleic acids, with different colors representing different nucleotides. (SEQ ID NO: 582)

FIG. 17 illustrates cleavage efficiency below a 1:10 DNA to RNP ratio.

FIG. 18 illustrates cleavage efficiency below a 1:3 DNA to RNP ratio.

FIG. 19 illustrates a wild type scaffold and variant scaffolds targeting the same gene within the factor XII gene.

FIG. 20 illustrates in vitro cleavage assay comparing GFP cleavage of the wild type scaffold to the variant scaffolds identified via SELEX. 2 ratios were used, a 1:1 ratio of 1 picomole of Cas9 RNP to 1 picomole of target DNA and a 1:3 ratio of 1 picomole of Cas9 RNP to 3 picomoles of target DNA.

FIG. 21 shows that CRISPR single guide RNAs (sgRNAs) are composed of two functional domains that must complement one another to support editing activity. The DNA binding domains are designed to be complementary to target sites of interest. Some of them (e.g. against Target 1-black) allow for proper folding of the sgRNA when appended to the wild type Cas9-aptamer binding domain resulting in a functional sgRNA that can form a sgRNA-Cas9-DNA complex to edit DNA (bottom left with properly folded aptamer domain (green)). Many other DNA binding domains (e.g. against Target 2) result in improper folding and a dysfunctional sgRNA that is unable to edit DNA (bottom right with red misfolded aptamer binding domain and purple DNA binding domain).

FIG. 22 shows a partially randomized library used in Guide SELEX for cleavage capable variant gRNA scaffolds. a). Guide SELEX was performed using a partially randomized degenerate pool library based on the canonical SpCas9 guide RNA sequence. Each position of the randomized region had a 58% chance of being the canonical nucleotide and a 14% chance of being any of the other 3 nucleotides. Standard Scaffold Sequence: SEQ ID NO: 578. b). Guide SELEX consisted of two parts: a selection for RNP formation or binding of the pool to Cas9 (1), and a separate TdT-based screen for variant guides permitting cleavage (2). TdT adds a poly(A) tail to the cleavage site which becomes a handle for capture by a biotinylated Oligo(dT) probe and streptavidin beads. c). A graph showing the percent of radiolabeled DNA recovered in the indicated fration. D). Cleavage of a Cy5-labeled DNA target by w.t., pool, or each round of gRNA library can be detected by Oligo(dT) capture of the fluorescently-labeled substrate/RNP complex on streptavidin beads and interrogation by flow cytometry.

FIG. 23 shows selected variant gRNA sequence differences from w.t. gRNA. a). The sites of base changes in variant gRNAs are shown in complex with SpCas9 viewed from two different angles (“Front” and “Back”). The degree of conservation between a given base and the w.t. scaffold is color coded as follows: 95-100% conserved in red, 90-95% conserved in orange, 75-90% in yellow, and 0-75% conserved in green. The features of both the scaffold and protein are as indicated. Constant regions of the library (gRNA spacer and stem loop 3) are shown in black, and the target DNA is shown in purple. b). The variability of selected functional variant gRNA sequences is mapped onto the w.t. gRNA sequence. A 100% identity means the base at that site remained unchanged among all sequences analyzed. Note that stem loop 3 was held constant for the selection and remains identical to the w.t. sequence. Individual sequences used in the analysis are shown with specific base changes indicated (SEQ ID NO: 583).

FIG. 24 shows selected gRNA variants display a range of cleavage activity in vitro and in cells. One hundred sequences representing significant nodes of a phylogenetic map (FIG. 29) as well as clones bearing interesting features were tested both in vitro and in HEK293-GFP cells for cleavage activity. Outside numbers are the sequence identities of each clone. Inside numbers tell the number of mutations varied from the w.t. sequence of each clone. The outer ring of boxes (colored in shades of red) signify those clones capable of cleavage in tissue culture cells, with intensity of color representing higher cleavage activity. Inner boxes show cleavage activity in in vitro tests. Black boxes in both rings represent no cleavage. Representatives from significant nodes were chosen for further investigation. b). Selected gRNA variants were tested for knockdown of GFP in 3 different cell lines constitutively expressing GFP: HEK293, HeLa and PC3. Of note, the HeLa-GFP cells express a destabilized, short-lived version of GFP. GFP expression was determined by flow cytometry on day 6 post-transfection, and data are shown as the percentage of cells that were GFP negative in the population. Data represent 3 replicates of each sample.

FIG. 25 shows different scaffold and targeting domain combinations yield a range of editing abilities. a). Ten selected variant gRNAs were re-targeted to other sites on the GFP gene, and GFP knockdown was assessed in HEK293-GFP and HeLa-GFP cells on day 6 post-transfection. The degree of GFP knockdown is shown for each variant with each target. White colored boxes represent no knockdown, black indicate 50% knockdown, and red represent >80% reduction in GFP. Variant 226 (b) and 232 (c) are shown in complex with SpCas9. Sites on the variant scaffolds that maintain sequence identity to the w.t. scaffold is shown in red, and sites that differ from the w.t. scaffold are colored in green. The features of both the scaffolds and protein are as indicated. Constant regions of the library (gRNA spacer and stem loop 3) are shown in black, and the target DNA is shown in purple. d) An overlap of the 226-Cas9 complex with the 232-Casp9 complex displays the contrasting mutational landscape between the variant guide RNAs. Sequence changes in variant 226 from the wild type are shown in yellow, and sequence changes of variant 232 are shown in blue. Both guides have a similar number of mutations within the different portions of the scaffold; however, the specific location and composition of these mutations differ. These changes result in altered cleavage activity between the two sequences, enabling scaffold 226 to cleave more efficiently against Target 10 and less efficiently against Target 5, while scaffold 232 displays the opposite cleavage pattern.

FIG. 26 shows selection for RNP formation does not necessarily yield cleavage capable guide variants. Clones from the RNP-forming/binding selection aligned with Geneious and rank ordered with FastAptamer. The top 60 clones were complexed with Cas9 and incubated with Substrate 1 (top 2 images) or Substrate 2 (bottom 3 images; see Supplementary Table 1) for 30 minutes at 37° C., as described in Materials & Methods. The reactions were treated with 1 uL of 20 mg/mL proteinase K, run on 3% LE-agarose gels stained with SYBR SAFE, and imaged using BioRad Image Lab software. Cleavage of the is detected by the appearance of lower band. Of the clones analyzed from the RNP formation/binding selection, only a few demonstrated significant cleavage, and fewer still led to cleavage on par with the w.t. scaffold. A functional screen was added to the selection process to enable selection of cleavage-capable variants.

FIG. 27 shows TdT-based capture of cleaved RNP complexes. Variant or w.t. gRNAs are complexed to SpCas9 and a radiolabeled or fluorescently labeled DNA substrate. Upon cleavage by Cas9, TdT adds a poly(A) chain to the PAM-distal DNA free single-stranded 3′ end. A biotinylated Oligo(dT) probe binds the poly(A) tail, and the whole complex can be captured with magnetic streptavidin coated beads. Captured complexes are analyzed by scintillation counting or flow cytometry. Shown is the scheme for Cy5 labeling of cleaved RNP complexes for analysis by flow cytometry.

FIG. 28 shows validation of RNP cleavage capture via TdT. Cleavage by the w.t. scaffold complexed with inactive “dead” SpCas9 or active SpCas9 was assessed by radiolabeled bead-based capture assays using TdT in an A-tailing reaction (FIG. 27). Capture of the radiolabeled substrate/RNP complex on streptavidin beads or in the wash fraction was determined by scintillation counting.

FIG. 29 shows phylogenetic mapping of select clones. Sequences from the selections were aligned with Geneious and frequency ranked using FastAptamer. Top ranking clones as well as clones bearing interesting features were grouped phylogenetically demonstrating variance from the w.t. scaffold sequence. For ease, only 1,000 clones are shown on this phylogenetic map.

FIG. 30 shows gRNA variants selected from the functional screen are largely capable of Cas9 mediated cleavage. Representative clones following the TdT functional screens were complexed with Cas9 and incubated with Substrate 2 (see Supplementary Table 1) for 30 minutes at 37° C., as described in Materials & Methods. The reactions were treated with proteinase K, run on 3% LE-agarose gels stained with SYBR SAFE, and imaged using BioRad Image Lab software. Cleavage of the 600 base pair Substrate 1 is detected by the appearance of a lower 300 bp band. Of the 200 clones analyzed from the functional selection, 109 led to cleavage comparable to that of the wild type scaffold. FIG. 31 shows binding of w.t. and variant 226 and 232 to Cas9 when targeted to 2 different sites. When directed to Target 5, all 3 scaffolds tested appear to bind similarly to Cas9, with variant scaffold 232 slightly higher than the w.t. scaffold. On the other hand, when directed to Target 10, variant scaffold 226 appears to have slightly higher affinity for Cas9. While the binding differences appear modest, the cleavage efficiencies of these scaffolds were more dramatic (FIG. 25) with trends matching the binding data. For these assays, gRNA scaffolds were end labeled using 20 U T5 Polynucleotide Kinase (NEB) and 20 Ci (5000 Ci/mmole) adenosine 5-[-32P]-triphosphate (GE Healthcare) at 37° C. for 1 hour. Radiolabeled RNAs were cleaned with Bio-Spin P30 columns (BioRad) and eluted in TE to remove unincorporated nucleotides. A constant trace amount of radiolabeled sgRNA scaffolds were incubated with decreasing levels of Cas9 to form a titration curve. The complexes were filtered in a double-filter nitrocellulose binding assay, read on a GE Storm 840 Phosphorimager, and the fraction of bound RNA and non-specific background corrections were conducted and assessed as previously described (Wong & Lohman, 1993, PNAS 90(12):5428).

FIG. 32 demonstrates the strategy for developing a starting library for selection of variant Staphylococcus aureus gRNAs based the disclosed methods.

FIG. 33 shows the starting DNA library mapped against wt Staphylococcus aureus Cas9 (saCas9) Grna (SEQ ID NO: 1).

FIG. 34 demonstrates the strategy for ligand evolution of CRISPR gRNAs for saCas9 using SELEX.

FIG. 35 demonstrates the aptamer-binding step of the disclosed methods.

FIG. 36 shows the ribonucleoprotein (RNP) and DNA binding round of the disclosed methods.

FIG. 37 shows that the SaCas9 assay preferentially pulls down the PAM proximal biotin labeled DNA and that a middle level of detergent was optimal for PAM proximal pull down.

FIG. 38 shows the DNA sequence used for pull-down (SEQ ID NO: 2).

FIG. 39 shows that all the tested processes described in FIG. 37 yielded gRNAs capable of cleaving DNA in combination with saCas9 in vitro.

FIG. 40 shows a schematic of the cleaving assay used in an exemplary agarose gel demonstrating the cleavage of gRNAs pools from selected rounds of the processes described in FIG. 34-36.

FIG. 41 shows sequencing results of each of the processes.

FIG. 42 shows results of cleavage assays for gRNAs isolated from the indicated processes described in FIG. 37 and demonstrates that process 1 variants produced cleavage products in combination with Cas9 and target DNA.

FIG. 43 shows cleavage of gRNA pooled variants from process 1 after 3 and 6 rounds and demonstrates increased cleavage of target DNA with pooled variant gRNAs from process 1 round 6 compared to process 1 round 3.

FIG. 44 shows target DNA cleavage by SaCas9 in complex with various gRNA variants discovered by the disclosed novel methods. In particular, variants (scaffold #s) 4-6 and 8-16 are able to cleave target DNA in combination with SaCas9.

FIG. 45 shows a mutation map of the variant Staph. a. gRNAs (From top to bottom: SEQ ID NOs: 547-563).

FIG. 46 shows a diagram of a method of testing the cleaving of GFP of the variant gRNAs in a cell line.

FIG. 47 shows the eGFP gene with the target site labeled at 130-155 and shows eGFP targeting by the Cas9-indicated gRNA complexes demonstrating cleavage of the target in each case in vitro. Target sequence (CCGGTGGTGCAGATGAACTT (SEQ ID NO: 577)).

FIG. 48 shows the results using the same gRNAs when they were introduced into a cell expressing target DNA (GFP), gRNAs 3, 5, and 14 showed comparable knockdown levels to the wild type gRNA. gRNAs 3, 5, and 14 relate to scaffolds from SEQ ID NOs: 550, 552 and 561.

### DETAILED DESCRIPTION OF THE INVENTION

Disclosed herein are novel methods of generating CRISPR genome editing agents using a combinatorial chemistry approach. As demonstrated in the Examples, a combinatorial library of potential novel guide RNA molecules on the order of 10¹⁵ was prepared and screened for those guide RNA molecules able to bind Cas9 and support Cas9-mediated cleavage of target DNA. Thereby, the inventors have discovered and optimized an inventive approach to developing reagents for targeted gene editing. In addition, the inventors have identified novel nucleic acids that can serve as guide RNAs for nucleic acid editing. Traditional approaches to CRISPR-based gene editing involve selecting a sequence complementary to the target region to be modified and inserting the sequence into existing gRNA “scaffolds” which comprise the elements that allow binding to Cas proteins and promote cleavage of the target, e.g., the tetraloop, stem loop 1, stem loop 2, and stem loop 3. The technologies disclosed herein revolutionize the existing approaches to the development of gene editing reagents by allowing one of skill in the art to not only select a target region to be modified, but also to develop entirely novel gRNAs to fine-tune the editing to the user's satisfaction. Furthermore, the inventors disclose herein novel gRNAs which may be used in place of existing gRNAs that are derived from gRNA sequences found in nature.

CRISPR (clustered regularly interspaced short palindromic repeats) loci are found in a wide range of bacteria and have now been shown to be transcribed to generate a family of targeting RNAs specific for a range of different DNA bacteriophage that can infect the bacterium. In bacteria that express a type II CRISPR/Cas system, these phage-derived sequences are transcribed along with sequences from the adjacent constant region to give a CRISPR RNA (crRNA) which forms a complex with the invariant trans-activating crRNA (tracrRNA), using sequence complementarity between the tracrRNA and an invariant region of the crRNA. This heterodimer, referred to as a guide RNA (gRNA), is then bound by the effector protein of the type II CRISPR/Cas systems, called Cas9. Cas9 has the ability to directly recognize a short DNA sequence called a protospacer adjacent motif (PAM). In the case of the commonly used Streptococcus pyogenes (Sp) Cas9 protein, the PAM site is 5′-NGG-3′. The Cas9 protein scans a target genome for the PAM sequence and then binds and queries the DNA for full 5′ sequence complementarity to the variable part of the crRNA. If detected, the Cas9 protein directly cleaves both strands of the target bacteriophage DNA˜3 bp 5′ to the PAM, using two distinct protein domains: the Cas9 RuvC-like domain cleaves the non-complementary strand, while the Cas9 HNH nuclease domain cleaves the complementary strand. This dsDNA break then induces the degradation of the phage DNA genome and blocks infection of the bacterium. Thus CRISPR/Cas based systems are both highly specific and allow retargeting to new genomic loci with variable efficiencies.

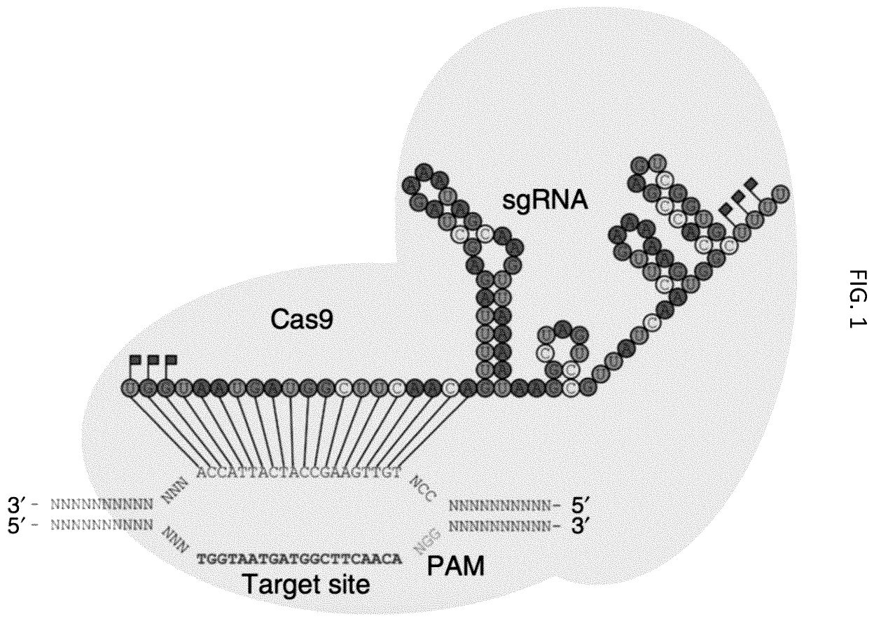

A key step forward in making the Cas systems more user-friendly for genetic engineering in human cells was the demonstration that the crRNA and tracrRNA could be linked by an artificial loop sequence to generate a fully functional small guide RNA (sgRNA)˜100 nt in length. (FIG. 1) Further work, including mutational analysis of DNA targets, has revealed that sequence specificity for Cas9 relies both on the PAM and on full complementarity to the 3′˜13 nt of the ˜20 nt variable region of the sgRNA, with more 5′ sequences making only a minor contribution. Cas9 therefore has an ˜15 bp (13 bp in the guide and 2 bp in the PAM) sequence specificity for targeting DNA.

CRISPR systems have been identified and characterized from many different bacteria and any of these Cas enzymes may be used in the methods described herein, for example, Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The Cas protein can be from any bacterial or archaeal species. For example, in some embodiments, the Cas protein is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella. For example Cas9 proteins from any of Corynebacter, Sutterella, Legionella, Treponema, Filifactor, Eubacterium, Streptococcus, Lactobacillus, Mycoplasma, Bacteroides, Flavobacterium , Sphaerochaeta, Azaspirillutn, Gluconacetobacter, Neisseria, Roseburia, Parvibaculum, Staphylococcus, Nitratifactor, Mycoplasma and Campylobacter may be used. In some embodiments, the Cas proteins have modified function, e.g., Cas nickase or catalytically dead Cas. In some embodiments, the Cas proteins are fused to another proteins which uses the CRISPR system to be targeted to a specific locus on DNA or RNA.

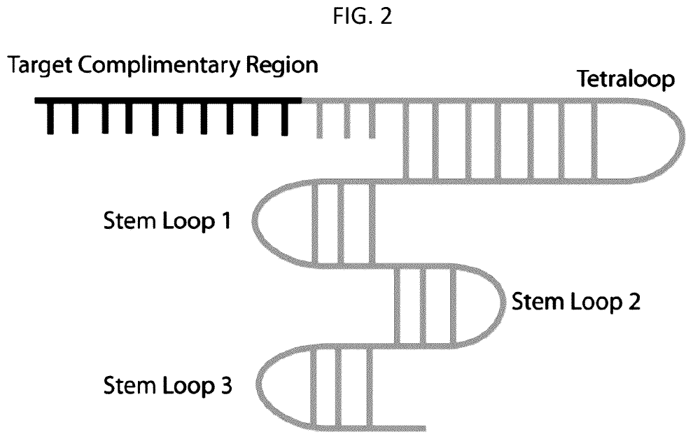

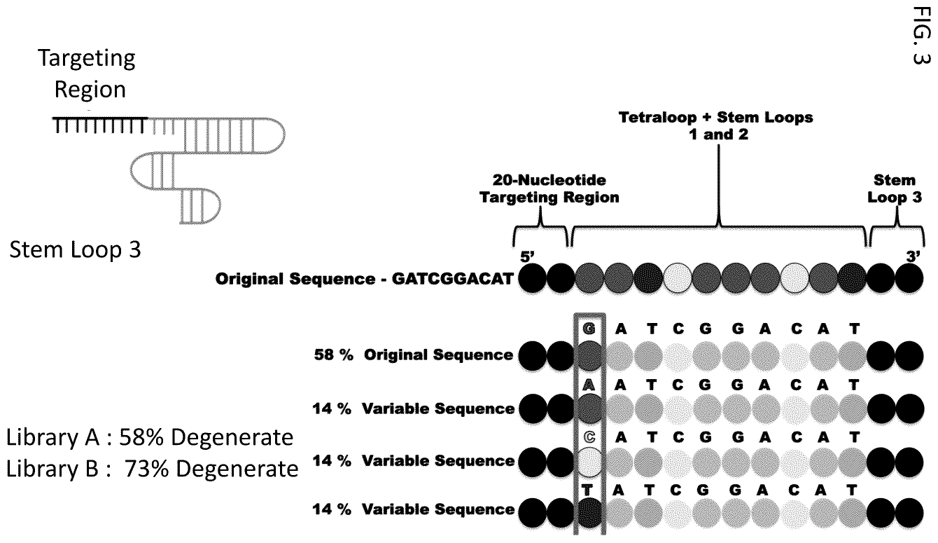

In the Examples, the inventors reduce to practice the novel methods with both Streptococcus pyogenes (Sp) and Staphylococcus aureus (Sa) CRISPR Cas9 systems, but other CRISPR systems may be used. As discussed above, Cas9 proteins rely on a distinct recognition site or PAM. The PAM for Sp Cas9 is 5′-NGG-3′, for Neisseria meningitides (Nme) it is 5′-NNNNGATT-3′ and for Staphylococcus aureus (Sa) the PAM is identified herein as 5′-NNGRRT-3′, where R is purine. Each has a distinct sgRNA scaffold sequence making up the 3′ portion of the single guide RNA. A representation of the scaffold for Sp guide RNA is shown in FIG. 2. The length of the target sequence specific 5′ portion of the sgRNA varies between the Cas9 enzymes as well. SpCas9 uses 18-20 nucleotide target sequences. NmeCas9 and SaCas9 use a 18-24 nucleotide target sequence.

In the CRISPR system, the Cas9 enzyme is directed to cleave the DNA target sequence by the sgRNA. The sgRNA includes at least two portions having two functions. The first portion is the DNA targeting portion of the sgRNA and it is at the 5′ end of the sgRNA relative to the second portion. The first portion of the sgRNA is complementary to a strand of the target sequence, referred to herein as a “template-conserved target complementary region”. The target sequence is immediately 5′ to the PAM sequence for the Cas9 on the target nucleic acid. Thus, the template conserved target complementary region is proximate to the PAM site, i.e., within less than 5 nucleotides, less than 4 nucleotides, less than 3 nucleotides, less than 2 nucleotides, 1 nucleotide away from the PAM site, or the template-conserved target complementary region may comprise the PAM site. The portion of the sgRNA that is complementary to the target sequence may be 10 nucleotides, 13 nucleotides, 15 nucleotides, 18 nucleotides, 20 nucleotides, 22 nucleotides or 24 nucleotides in length or any number of nucleotides between 10 and 30. The portion of the sgRNA complementary to the target sequence should be able to hybridize to the sequences in the target strand and is optimally fully complementary to the target sequence. The exact length and positioning of the complementary portion of the sgRNA will depend on the Cas9 enzyme it is being paired with. The Cas9 enzyme selected will require that the sgRNA is designed specifically for use with that enzyme and will control the design of the sgRNA.

The second portion of the sgRNA which is at the 3′ end of the sgRNA is the scaffold that interacts with the Cas protein and which is specific for each Cas protein.

Although the Examples demonstrate the generation of sgRNA suitable for use in DNA cleavage or editing, the methods disclosed herein may be readily extended to the generation of sgRNA suitable for use in RNA cleavage or editing, such as with a CRISPR-Cas13 system (Cox, David B. Science 358(6366) 1019-1027 (2017).

The combinatorial methods described herein allow for the generation of novel guide nucleic acids, including novel scaffold sequences, and identification of candidate guide nucleic acids based on having a desired property. Suitably the desired property may be selected for binding affinity to the desired Cas protein, cleavage activity, or any other suitable property. Suitably, the combinatorial methods described herein may allow for generation of novel sgRNAs that have both high binding affinity for a Cas protein and high cleavage activity.

### Methods for Generating Guide Nucleic Acids

Accordingly, in one aspect of the current disclosure, methods for generating guide nucleic acids that bind a Cas protein are provided. In some embodiments, the methods comprise (a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) in the target nucleic acid and wherein the template-randomized scaffold comprises a degenerate nucleic acid 5′ portion and an invariant 3′ end, (b) partitioning candidate guide nucleic acids having an increased binding affinity to the Cas protein from candidate guide nucleic acids having a reduced binding affinity to the Cas protein; and (c) amplifying the candidate guide nucleic acids having the increased binding affinity to the Cas protein to generate a candidate mixture enriched for candidate guide nucleic acids having binding affinity for the Cas protein.

### Preparing a Mixture of Guide Nucleic Acids:

The mixture generally includes regions of fixed sequences (i.e., each of the members of the candidate mixture contains the same sequences in the same location, also called invariant sequences) and regions of randomized sequences. The fixed sequence regions are selected either: a) to assist in the amplification steps described below such as by acting as a primer binding region for PCR amplification; b) to mimic a sequence known to bind to the target; or c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

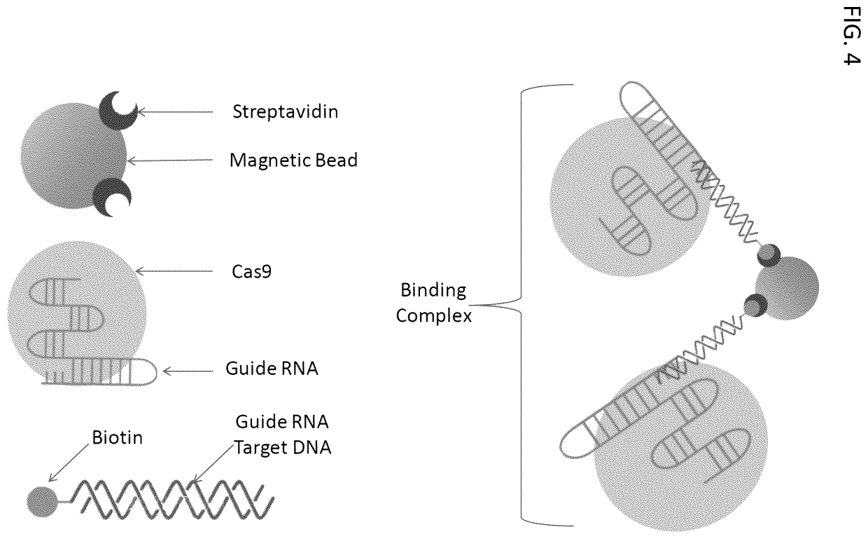

As shown in FIG. 3, the mixture of candidate nucleic acids may be prepared by conserving a target complementary region configured to hybridize to a double-stranded DNA proximate to a PAM, i.e., “the template-conserved target complementary region”, and randomizing some or all of the scaffold portion. In some embodiments, the scaffold has nucleotides that are randomly selected, i.e., “randomized”, and may comprise a degenerate nucleic acid 5′ portion comprising, e.g., randomized tetraloop and stem loops 1 and 2, and an invariant 3′ end, e.g., stem loop 3 (See FIG. 2). This strategy allows for tailoring the interaction or binding affinity of the candidate nucleic acid for the Cas protein, such as Cas9, while conserving the targeting function of the guide nucleic acid. In the Examples, stem loop 3 was conserved while allowing for randomization of the tetraloop as well as stem loops 1 and 2. However, in other embodiments, stem loop 3 may be completely or partially randomized. However, critically, the entire sequence should not be randomized as some degree of a priori knowledge of the sequences in the mixture must be had to design reagents, i.e., primers, to amplify nucleic acids that can successfully bind to Cas proteins. The fixed or invariant portion may include additional nucleotides added to the end of the gRNA extending beyond stem loop 3 as an additional alternative. In the Examples, guide nucleic acids for S. pyogenes and S. aureus Cas9 served as templates for randomization, but other guide nucleic acids may be selected for this purpose depending on the Cas9 protein to be used. Thus, in some embodiments, the Cas9 endonuclease is Streptococcus pyogenes Cas9 endonuclease or functional variant thereof with, e.g., greater than 90% sequence identity, greater than 92% sequence identity, greater than 95% sequence identity, or greater than 98% sequence identity to Streptococcus pyogenes Cas9 endonuclease and is also able to mediate gRNA based template cleavage. In some embodiments, the Cas9 endonuclease is Staphylococcus aureus Cas9 endonuclease or functional variant thereof with, e.g., greater than 90% sequence identity, greater than 92% sequence identity, greater than 95% sequence identity, or greater than 98% sequence identity to Staphylococcus aureus Cas9 endonuclease and is also able to mediate gRNA based template cleavage. The Cas9 used in the methods should be a functional Cas9 capable of forming the Cas9 complex with the gRNA and target nucleic acid. In some embodiments, the Cas9 also mediates cleavage of at least one strand or both strands of the target nucleic acid.

Suitably, the candidate guide nucleic acids may be comprised of naturally occurring, non-naturally occurring, or any combination of naturally occurring and non-naturally occurring ribo- and deoxyribonucleotides. Suitably, the non-naturally occurring nucleotides may have nucleotides with base modifications (e.g., 2-thiouridine, N6-methyladenosine, or pseudouridine), backbone modifications (e.g., phophorothioate or boranophosphate), sugar modifications (e.g., 2′-OMe, 2′-F, LNA, 2′-NH₂), 5′ and/or 3′ covalent linkages to a variety of molecular entities, or any combination thereof. The molecular entities covalently linked to the 5′ and/or 3′ end may include detection tags (e.g., biotin), labels (e.g., fluorescent dyes), proteins, lipids (e.g., cholesterol or derivatives thereof), PEG, or any combination thereof. Guide nucleic acids with base modifications may result in guide nucleic acids having increased nuclease resistance, increased complex stability, improved gene editing function, allow for in vivo expression or delivery, provide novel molecular interactions, or any combination thereof depending on the modifications selected.

The mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target, i.e., “increased binding affinity”. As used herein a Cas protein may be a protein or polypeptide capable of being used in a CRISPR system or representative of a CRISPR system. In some embodiments, the Cas protein is a naturally occurring or non-naturally occurring Cas9 endonuclease having binding affinity for a guide nucleic acid and double-stranded DNA cleavage activity proximate to a PAM. In other embodiments, the Cas protein may be a protein or polypeptide having representative of binding interactions with the guide nucleic acid as a naturally occurring or non-naturally occurring Cas9 endonuclease but lacking cleavage activity. Accordingly, one advantage of the present technology is the ability to tailor guide nucleic acids to new Cas9 endonucleases and optimize their ability to target various DNA sequences.

In some embodiments, the methods, mixtures, complexes, gRNA sequences of the instant disclosure are suitable for optimizing the function of systems based on Cas proteins with modified enzyme activity, e.g., Cas nickases or catalytically dead Cas (dCas). In some embodiments, the disclosed methods may be used to generate improved gRNAs for methods utilizing Cas nickases, e.g., RNA editing with Cas-adenosine deaminase acting on RNA (ADAR) fusions, epigenetic modification, e.g., methylation, control of expression, base editing, prime editing, etc. For example, prime editing requires that a prime editing gRNA (pegRNA) comprise both the targeting sequence and a template sequence to be introduced into the target locus of the genome. Thus, pegRNAs possess increased complexity compared to standard Cas gRNAs. Thus, in some embodiments, the disclosed methods are used to generate complex gRNAs, e.g., pegRNAs for use in prime editing.

### Partitioning Sequences

The nucleic acids with the highest affinity for binding to the Cas protein are partitioned from those nucleic acids with lesser affinity. Because only a small number of sequences (and possibly only one molecule of nucleic acid) corresponding to the highest affinity nucleic acids exist in the mixture of candidate nucleic acids, it is generally desirable to set the partitioning criteria so that a significant amount of the nucleic acids in the candidate mixture (approximately 5-50%) are retained during partitioning.

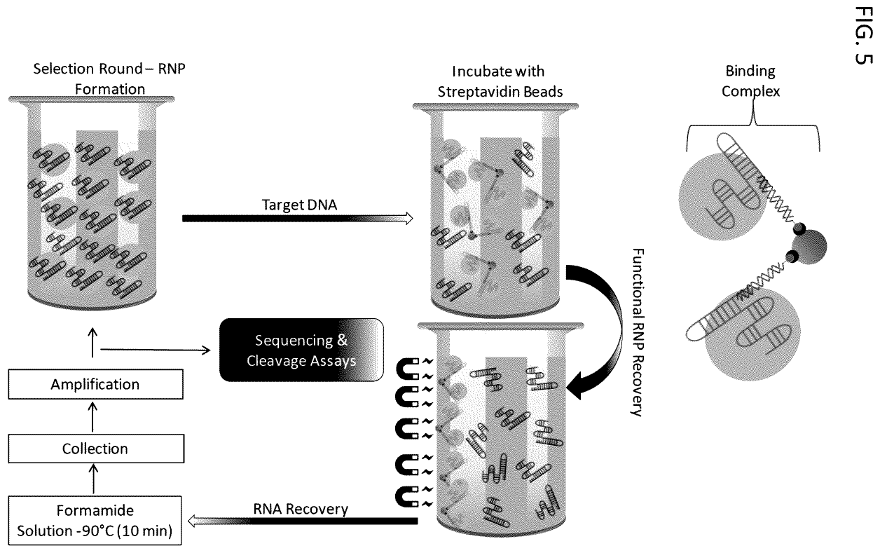

FIG. 4 illustrates an exemplary molecular system for partitioning candidate guide nucleic acids. The system relies on magnetic beads operably connected to streptavidin and biotin labeled probes (target nucleic acids). As shown in FIG. 4, the biotin labeled probes comprise target DNA complementary to the candidate guide nucleic acid template-conserved target complementary region, which allows for the partitioning of the Cas protein/guide nucleic acid binding complex. Such an approach will allow for enrichment of the mixture for guide nucleic acids having increased binding affinity for a Cas protein.

Those nucleic acids selected during partitioning as having the relatively higher affinity to the target are then amplified to create a new candidate mixture that is enriched in nucleic acids having a relatively higher affinity for the target.

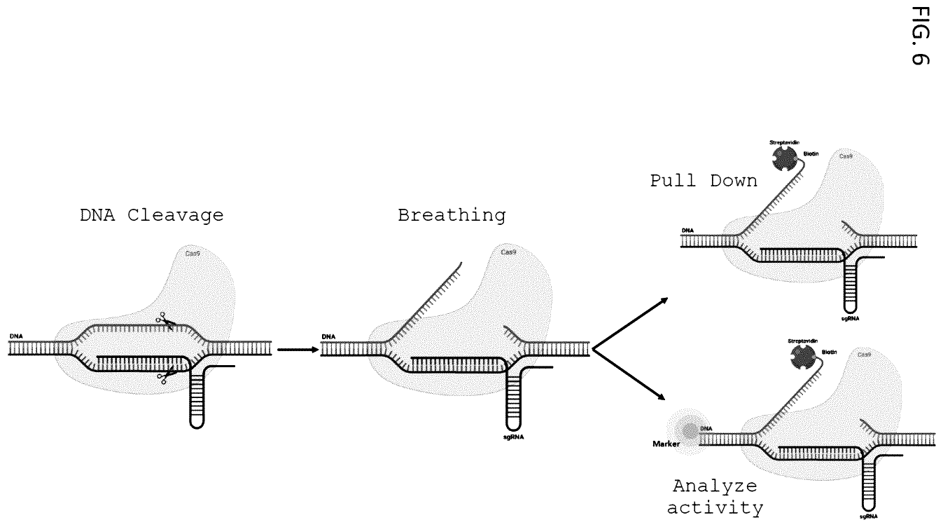

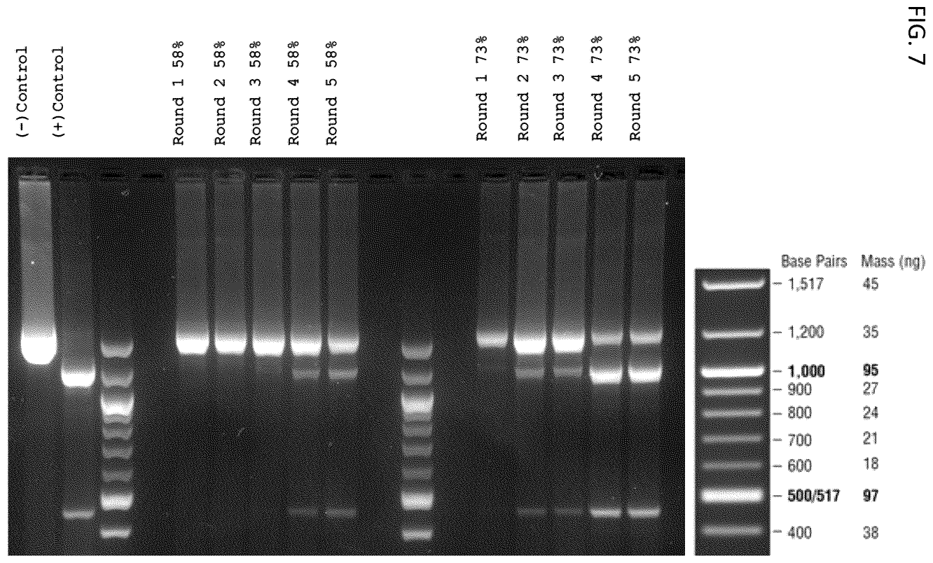

By repeating the partitioning and amplifying steps above, the newly formed candidate mixture contains fewer and fewer unique sequences, and the average degree of affinity of the nucleic acids to the target will generally increase. A summary of the general process is illustrated in FIG. 5.

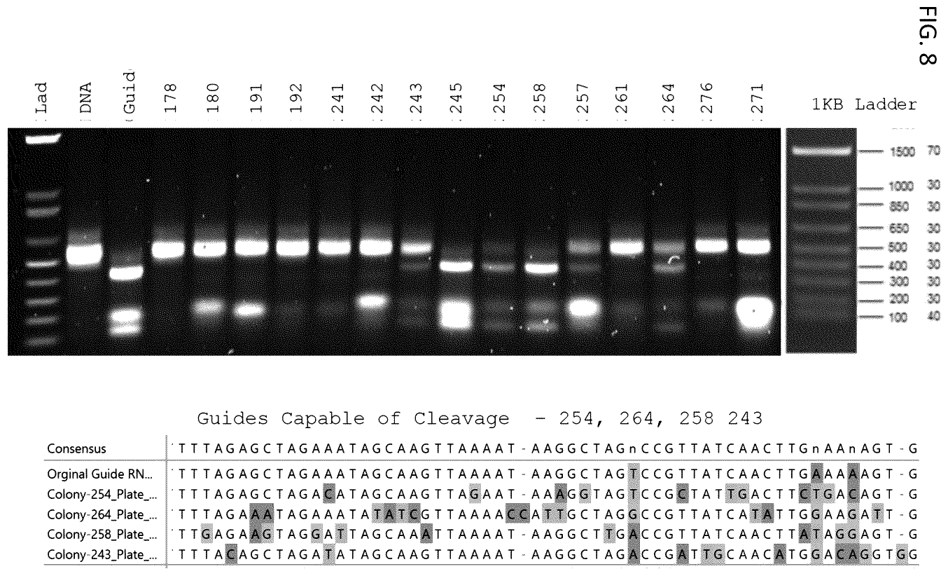

An alternative embodiment for enriching the candidate mixture is illustrated FIG. 6. Such an embodiment allows for enrichment based on cleavage activity of the gRNA-Cas complex. Accordingly, in another aspect of the current disclosure, methods for generating guide nucleic acids that allow cleavage of a double-stranded nucleic acid target when in complex with a Cas protein are provided. In some embodiments, the methods comprise: (a) contacting a Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) in the target nucleic acid and wherein the template-randomized scaffold comprises a degenerate nucleic acid 5′portion and an invariant 3′ end, thereby forming one or more Cas protein-candidate guide nucleic acid complexes; (b) partitioning candidate guide nucleic acids having an increased Cas complex cleavage activity by selecting the Cas protein-candidate guide nucleic acid complexes having a free single-stranded DNA 3′ end from candidate guide nucleic acids having a reduced Cas complex cleavage activity; and (c) amplifying the candidate guide nucleic acids having the increased Cas complex cleavage activity to generate a candidate mixture enriched for candidate guide nucleic acids having Cas complex cleavage activity.

This partitioning strategy employs the free single-stranded DNA 3′ end, or simply the free end, that occurs when the three components of a Cas complex, the Cas protein, guide nucleic acid, and double stranded DNA target nucleic acid, associate with each other in such a way as to accomplish cleavage of the DNA. Upon cleavage, the free end may be labeled such that partitioning may be accomplished. This approach selects not only for Cas binding but binding in a manner that is compatible with DNA cleavage. In some embodiments, the target DNA is labeled at the 3′ end to prevent TdT from adding terminal nucleotides prior to the cleavage of the target DNA by a Cas protein. The inventors observed that while both the above-described strategies may yield novel gRNAs that allow cleavage of target DNA by Cas proteins, one strategy may be more suitable for a particular gRNA/Cas system than the other. By way of example, but not by way of limitation, the inventors observed that simply selecting for binding of gRNAs to target DNA and Cas9 in the S. aureus system was sufficient to generate novel gRNAs capable of mediating Cas9 cleavage of the target. By contrast, generation of novel gRNAs capable of cleavage in the S. pyogenes system required that the selection be performed based on cleavage of target DNA, not simply based on binding of gRNAs to SpCas9.

In one embodiment, the free end is labeled with a detectable label. Such labeling may be accomplished when the candidate mixture also comprises a polymerase and a labeled nucleotide. Suitably, the polymerase may be selected from terminal deoxynucleotidyl transferase (TdT). TdT catalyzes the addition of nucleotides to the 3′ terminus of a DNA molecule. Unlike most DNA polymerases, it does not require a template. The preferred substrate of this enzyme is a 3′-overhang, but it can also add nucleotides to blunt or recessed 3′ ends. Suitably, the labeled nucleotide may have a detectable label operably attached thereto. Biotin-16-aminoallyl-2′-dATP is used in the Examples to add a poly-A tail to the PAM distal free 3′ cleaved strand, but other labeled nucleotides may also be used to label the free end of the cleaved DNA. Exemplary labels include, but are not limited to, fluorescent labels, enzyme labels, epitope tags, biotin, and nucleotide sequences, e.g., barcodes. As used herein, “barcodes” refer to known, unique sequences of nucleotides that are distinct and can be used to positively identify a sequence which comprises the barcode. Barcodes are also capable of hybridizing to a complementary nucleic acid. The label may be used in the partitioning step of the method to interact with a binding partner or label functional complexes. If a poly-A tail is added to the free 3′ end, then a biotinylated poly-dT oligonucleotide may be hybridized to complex and the biotin used to partition the Cas complex via its interaction with avidin or streptavidin.

Enriching a Candidate Mixture of gRNAs

In some embodiments, the candidate mixture enriched for candidate guide nucleic acids having binding affinity for the Cas protein is provided by: (i) contacting the Cas protein with the candidate guide nucleic acids and the target nucleic acid, (ii) partitioning candidate guide nucleic acids of step (i) having an increased binding affinity to the Cas protein from candidate guide nucleic acids having a reduced binding affinity to the Cas protein; and (ii) amplifying the candidate guide nucleic acids of step (i) having the increased binding affinity to the Cas protein to generate the candidate mixture enriched for candidate guide nucleic acids having binding affinity for the Cas protein. As used herein, “amplifying the candidate guide nucleic acids” refers to increasing the number of copies of the candidate guide nucleic acids that have been partitioned based on either Cas binding or participation in successful Cas-mediated cleavage of target DNA. In some embodiments, amplification of candidate guide nucleic acids is accomplished by reverse transcribing the candidate gRNAs with, e.g., MMLV or AMV reverse transcriptase, to yield single-stranded cDNA, amplifying the cDNA by polymerase chain reaction (PCR) using primers specific for the DNA sequences corresponding to the 5′ and 3′ ends of the gRNA to generate amplified, double-stranded DNA, which may be transcribed into gRNAs which may then be subjected to further rounds of selection according to the methods disclosed herein.

The methods disclosed herein allow for the generation of guide nucleic acids having increased or decreased Cas-complex cleavage activity relative to the template (i.e. the standard gRNA used with a particular Cas). Cas-complex cleavage activity may be measured by the percentage of cleavage of the target nucleic acid by methods such as disclosed in the Examples. Other methods for determining Cas complex cleavage activity may also be used. Cas-complex cleavage activity should be compared to the template under substantially similar environmental conditions, such as substantially similar in vitro, in vivo, or ex vivo environments. In some embodiments, the Cas-complex cleavage activity is increased at least 10%, 20%, 30%, 40%, 50%, or more relative to the template. In other embodiments, the Cas-complex cleavage activity is decreased at least 10%, 20%, 30%, 40%, 50%, or more relative to the template.

A notable advantage of the presently disclosed technology is that it allows for the generation of guide nucleic acids that are tailored to a particular environment. Accordingly, guide nucleic acids generated with the technology disclosed herein may have increased Cas-complex cleavage activity relative to the template in some environments and decreased Cas-complex cleavage activity relative to the template in other environments. This allows for the generation of guide nucleic acids that may be used for cell-specific or tissue-specific applications. As used herein, “cell-specific” or “tissue-specific” means that that the Cas-complex activity in a particular cell or tissue is at least 25% greater than other cells or tissues, suitably at least 50% greater than other cells or tissues. This also allows for the generation of guide nucleic acids where Cas-complex cleavage activity may be modulated by the presence or absence of one or more different compounds, such as miRNA.

In some embodiments, the guide nucleic acid allows for cleavage activity greater than 80%, 85%, 90%, 95%, or more under particular environmental conditions.

In other embodiments, the guide nucleic acid allows for cleavage activity of less than 20%, 15%, 10%, 5%, or less under particular environmental conditions.

Another notable advantage of the presently disclosed technology is that it allows for the generation of guide nucleic acids that are tailored to the particular nucleic acid target. A selected template guide nucleic acid has the potential to interact with the targeting region, forming unwanted secondary structures that inhibit the functionality of the Cas protein, or more particularly Cas RNP complex. Without being bound by any theory or mechanism, it is believed that unwanted interactions between the guide nucleic acid and the target nucleic acid may explain why cleavage activity at some target sites is low, which may be characterized as a cleavage percentage below 60%, below 50%, or below 40%. Gene editing may be significantly improved at genomic sites with low cleavage efficiency when the poor editing outcome is caused by intramolecular interactions between the template-conserved target complementary region and the scaffold sequence. As many potential target sites for Cas9 and other Cas proteins are not efficiently cleaved, it is not uncommon to screen 10 or more sites to identify a Cas9-RNP that is fairly efficient at cleaving the target. The presently disclosed technology allows for the generation of novel guides optimized for a particular target site and this will greatly expand the number of targetable sites that can be efficiently edited in the genome.

In some embodiments, the guide nucleic acids generated by the methods disclosed herein may have a functional site. As used herein, a functional site has a function independent of the guide nucleic acids' ability to bind a Cas protein and guide the Cas protein to a target nucleic acid. The guide sequences generated by the methods disclosed herein that possess full functionality may be used to rationally identify, design, or construct guides that have these functional sites built into them while still maintaining the structure and functionality of the guide. In some embodiments, the guide nucleic acids may be generated by the use of a template-conserved functional site, such as a template-conserved miRNA binding domain or a template-conserved miRNA domain.

In some embodiments, the functional site may be a miRNA or other regulatory domain. Such a guide nucleic acid may have a use in regulation of cellular functions via RNA silencing and post-transcriptional gene expression. Utilizing the variation discovered within the cleavage capable sequences, micro-RNA sites may be able to be built into the guide itself or enhance existing ones.

In some embodiments, the functional site may be a miRNA binding or other binding domain. Such a guide nucleic acid may allow for competitive inhibition in a particular environment. In other embodiments, the guide nucleic acid is selected such that it doesn't have a miRNA binding or other binding domain. Identification of active guides that do not have complementarity to miRNAs or other compounds capable of binding the guide in particular cells to create more active editors. This approach would enable the regulation of Cas cleavage profiles within a given cell type and/or temporarily alter cellular functions by giving the guide nucleic acid Cas-independent siRNA like functions without significantly altering the cleavage activity of the Cas9 ribonucleoprotein complex itself. Significant differences may exist in cleavage activity depending on the target cell type in comparison to the wild type gRNA sequence. Some guides generated with the methods described herein have very little cleavage activity in one cell type while displaying cleavage activity on par with the template in others. In some embodiments, this difference may be due to the alteration of micro-RNA binding sites within guides interfering with micro-RNAs of the cell. Use of a binding domain allows for cell or tissue specific activity. For example, miRNA-122 is one of the few micro RNAs highly specific for liver expression and it is one of the highest expressed micro-RNAs in the human body. Roughly 60-70% of micro RNAs in the liver consist of miRNA-122. The guide nucleic acid may be designed to have a site complementary to miRNA-122. The purpose of this is to inhibit guides in a tissue specific fashion utilizing the micro-RNAs that are highly expressed and tissue specific. A complementary sequence in high abundance will be sufficient to inhibit the guide nucleic acids function. This allows for Cas regulation systems revolving around cell and tissue specific expression to be built that either supplement or antagonize endogenous micro-RNA activity.

In some embodiments, the functional site may be a label for detecting or monitoring activity. For example, a guide may be designed to contain sequences targeted to GFP. Guides that contain a siRNA sequence targeted towards GFP should be able to knock down the expression of GFP via sequestration and degradation of the GFP mRNA transcript. This will allow for assaying functionality.

### Guide Nucleic Acids

In another aspect of the current disclosure, guide nucleic acids are disclosed. In some embodiments, the guide nucleic acids comprise a template-conserved target complementary region and a template-randomized scaffold, wherein the template-conserved target complementary region is configured to hybridize to a double-stranded nucleic acid target proximate to a protospacer adjacent motif (PAM) and wherein the template-randomized region has binding affinity for a Cas protein. In some embodiments, the guide nucleic acids comprise any one of the RNAs according to Table 1, 2 or 3.

According to the methods described herein, guide nucleic acids may be prepared. Exemplary guide nucleic acids generated and identified by the disclosed methods are shown in Tables 1-3. The sequences shown in Tables 1-3 include DNA generated by reverse transcription of the RNA candidates used in the Examples for partitioning, as well as the gRNA sequences themselves.

Novel Mixtures Comprising gRNAs

In another aspect of the current disclosure, mixtures are provided. In some embodiments, the mixtures comprise more than one candidate guide nucleic acid (gNA), the candidate guide nucleic acids having a common template-conserved target complementary region and each candidate guide nucleic acid having a distinct template-randomized scaffold, wherein the template-conserved target complementary region is configured to hybridize to a double-stranded DNA proximate to a protospacer adjacent motif (PAM) and wherein the template-randomized scaffold has binding affinity for a Cas protein. As used herein, “mixtures”, refer to combinations of gNAs (comprising both gRNAs and DNA complements thereof, i.e., gDNAs).

In some embodiments, the mixtures are candidate mixtures and comprise more than one candidate guide nucleic acids, the candidate guide nucleic acids having a common template-conserved target complementary region and each candidate guide nucleic acid having a distinct template-randomized scaffold, wherein the template-conserved target complementary region is configured to hybridize to a double-stranded DNA proximate to a protospacer adjacent motif (PAM) and wherein the template-randomized scaffold has binding affinity for a Cas protein.

In some embodiments, the mixtures further comprise a polymerase and a labeled nucleotide. In some embodiments, the polymerase is a terminal deoxynucleotidyl transferase (TdT). In some embodiments, the labeled nucleotide is biotin-16-aminoallyl-2′-dATP. In some embodiments, the candidate mixture is enriched for candidate guide nucleic acids having binding affinity for a Cas protein and/or Cas complex cleavage activity. In some embodiments, the candidate mixture was made by the methods disclosed herein or the mixtures are for use in the methods disclosed herein. In some embodiments, at least one of the candidate guide nucleic acids is selected from the guide nucleic acids in Table 1, Table 2, or Table 3.

### Cas Complexes

In another aspect of the current disclosure, Cas complexes are provided. In some embodiments, the Cas complexes comprise: (a) a Cas protein, (b) a candidate guide nucleic acid, the candidate guide nucleic acid comprising a template-conserved target complementary region and a template-randomized scaffold having binding affinity for the Cas protein; and (c) a cleaved target nucleic acid, the cleaved target nucleic acid comprising a free single-stranded labeled 3′ end. The Cas protein may, suitably, be any Cas protein or any Cas protein yet to be discovered. However, as discussed above, the inventors have exemplified the use of Cas9, specifically S. pyogenes and S. aureus Cas9. In some embodiments, the free single-stranded labeled 3′ end of the target nucleic acid is modified, e.g., biotinylated.

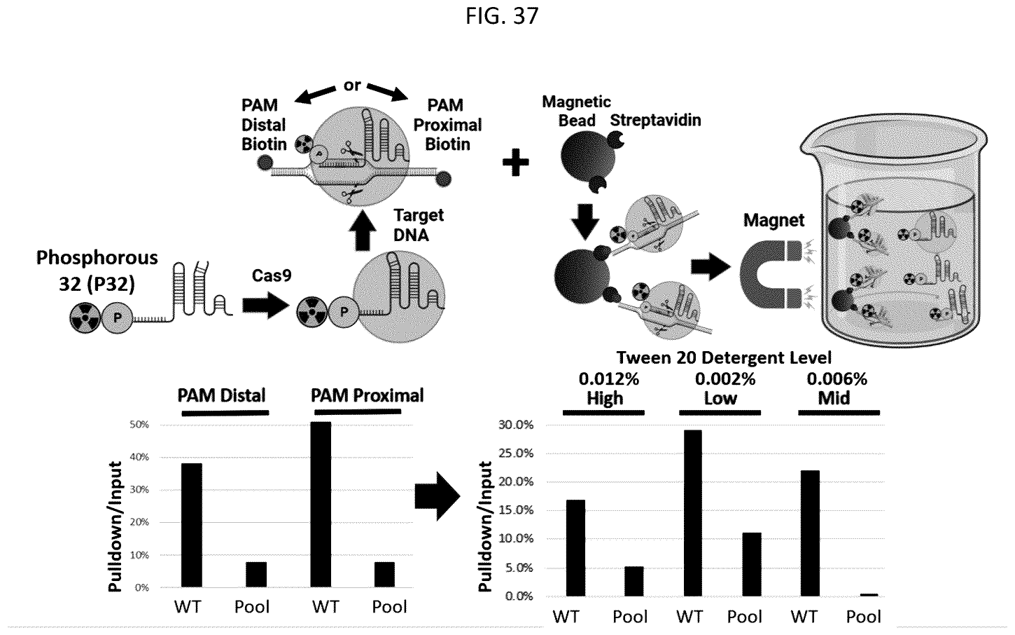

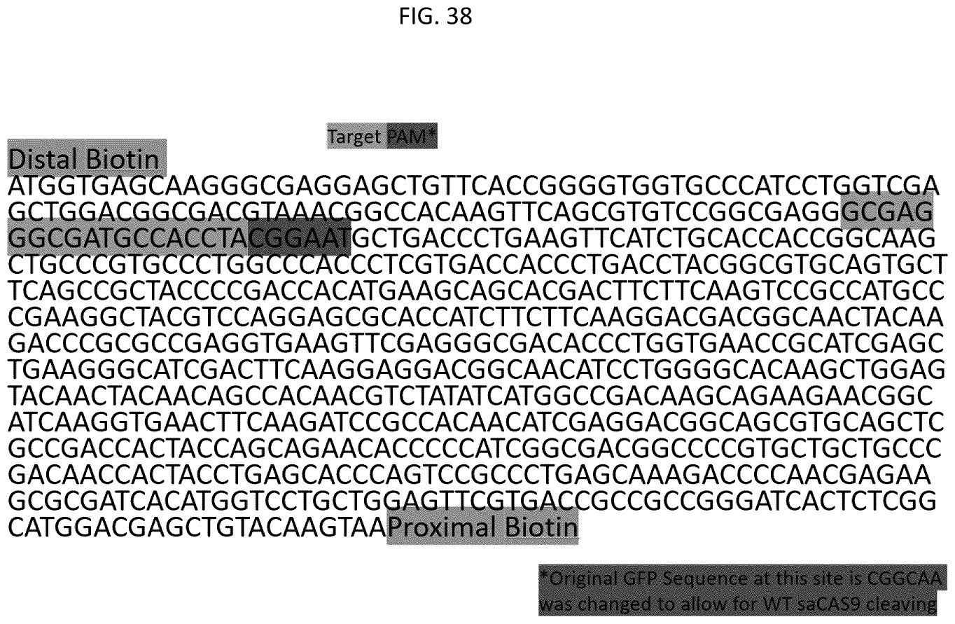

As discussed above, Cas proteins, e.g., Cas9, exist in a complex with gRNAs and the target nucleic acid even after the Cas protein has enzymatically cleaved the target nucleic acid. Interestingly, a feature of this post-cleavage complex is the presence of a free single-stranded 3′ end of the target nucleotide which is available for modification as well as the two ends of the target nucleic acid. Thus, in some embodiments, the cleaved target nucleic acid further comprises a second label. The inventors discovered that the particular Cas protein selected correlates with enhanced labeling of either the PAM proximal or the PAM distal end of the target nucleic acid as shown in FIG. 37. For example, the inventors demonstrate in FIG. 37 that a complex comprising S. aureus Cas9 tends to comprise labeled PAM proximal strand, while in Example 2, the inventors demonstrate that S. pyogenes Cas9 complexes tend to comprise labeled PAM distal strands. In some embodiments, the complexes comprise one or more candidate guide nucleic acids found in Table 1, Table 2, or Table 3.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Oligonucleotide Library Generation

The DNA template for the guide library contained a 60 nt variable region flanked by two constant primer binding regions consisting of a 19 nt GFP targeting sequence at the 5′ end and stem loop 3 of the guide RNA scaffold at the 3′ end (5′-GCGAGGGCGATGCCACCTA (SEQ ID NO: 3)-N60-GGCACCGAGTCGGTGCTTTT (SEQ ID NO: 4)-3′). The variable region was positionally biased towards the S. pyogenes Cas9 wild type guide RNA scaffold sequence. Each position was synthesized with a nucleotide pool consisting of the canonical nucleotide found within the standard guide RNA scaffold (58% composition) and an equimolar mix of the remaining 3 nucleotides (14% each). The primers (5′ primer: 5′-TAATACGACTCACTATAGGCGAGGGCGATGCCACCTA-3′ (SEQ ID NO: 5) and 3′ primer: 5′-AAAAGCACCGACTCGGTGCC-3′ (SEQ ID NO: 6)) and the template library were ordered from Integrated DNA Technologies (IDT). The DNA library was generated by annealing 1 nmol of the template oligonucleotide to 1.5 nmol of the 5 primer in 10 mM Tris-HCl pH 8.0 and 10 mM MgCl2 at 95° C. for 5 minutes and then was snap-cooled on ice for 5 minutes. Exo Klenow (New England Biolabs) was used to create a double stranded DNA fragment which was then phenol-chloroform extracted, desalted and concentrated in TE pH 8.0 with an 10K NMWL Amicon Ultra Centrifugal Filter Unit (Millipore). In vitro RNA transcriptions were conducted with an equimolar NTP mix (TriLink BioTechnologies) using a modified T7 polymerase (previously described in Sousa and Padilla, 1995; Fitzwater and Polisky, 1996; Padilla and Sousa, 1999) in a buffer composed of 40 mM Tris [pH 8], 5 mM DTT, 1 mM spermidine, 0.01% Triton X-100, 50 mg/ml PEG-8000 and 25 mM MgCl2. Following an overnight incubation at 37° C., transcription reactions were treated with deoxyribonuclease I (DNase I)/RNase-free (Sigma Aldrich), phenol-chloroform extracted and electrophoresed on a 12% acrylamide, 7 M urea, 0.5 M Tris borate EDTA (TBE) gel. The resulting RNA library was excised, eluted in TE pH 8.0 at 4° C., and desalted using a 10k Amicon Ultra Centrifugal Filter Unit.

In Vitro Procedure to Generate Novel Aptamers that Bind Cas9

The initial rounds of selection relied on affinity capture onto magnetic beads to enrich for sequences within the RNA library that bound to S. pyogenes Cas9. Primers (5′-biotin-TGTGCTGCAAGGCGATTAAG-3′ (SEQ ID NO: 7), 5-AAGTCGTGCTGCTTCATGTG-3′ (SEQ ID NO: 8)) were used to amplify biotinylated and non-biotinylated fragments of eGFP from a plasmid (gfap-EGFP-zebrafish, Addgene #65564). Oligonucleotides were ordered from IDT. 1 picomole of the biotinylated DNA fragment containing the eGFP target was incubated with 1 microliter of magnetic streptavidin beads (Thermofisher #65001), incubated overnight at 4° C. with rotation, and washed 3 times in cleavage buffer (50 mM Tris-HCL pH 7.9, 100 mM NaCl, 2 mM MgCl2, 0.01% bovine serum albumin (BSA), 0.05% Tween 20). For rounds 1 and 2, 100 picomoles of Cas9 was bound to the guide RNA library at an equimolar ratio in cleavage buffer and incubated at room temperature for 20 minutes. The potential ribonucleoprotein (RNP) complexes and streptavidin-DNA complexes were incubated at 37° C. for 1 hour and washed 3 times in cleavage buffer. Within this time frame, Cas9 remains stably bound to its target sequence following cleavage, an inherent property that enables for the preferential isolation and amplification of those sequences within the library that are capable of complexing with Cas9 and its subsequent DNA target. The RNA was extracted from the RNP-DNA complex by phenol:chloroform:isoamyl alcohol (25:24:1) extraction and subsequent ethanol precipitation. Half of the extracted RNA was reverse transcribed (Reverse Transcriptase AMV, Sigma-Aldrich) with 20 picomoles of the 3′ primer, 0.5 nanomoles dNTPs, and 20 units of AMV Reverse Transcriptase in the supplied buffer. The reaction was then PCR-amplified with the 5′ and 3′ primers (50 μL RT reaction, 0.5 nanomoles each primer, and 0.25 millimolar of dNTPs). A QIAquick PCR Purification Kit (Qiagen) was used to purify the PCR products, which were then used to for RNA amplification, as described above, to generate pool for the next round of SELEX for Cas aptamers.

Next, the inventors sought to identify those aptamers that can serve as functional aptamer-scaffolds that support Cas9-mediated cleavage of DNA. Following DNA cleavage, Cas9 holds on to three of the four ends of the target DNA fragment created at the cut site and releases the PAM-distal non-target strand from the RNP-DNA complex. The released strand can then be used to isolate the intact RNP-DNA complex and separate out novel aptamer-guides within the SELEX library that retain cleavage functionality from those that are incapable. This cleavage property of Cas9 was utilized for rounds 3 through 5 of our functional guideRNA selection. 200 picomoles of the RNA library was incubated with 0.1 millimolar dideoxy NTP's (dNTP) at an equimolar ratio and 100 units of terminal deoxynucleotidyl transferase (TdT, Sigma-Aldrich) in the supplied buffers. Following incubation at 37° C. for 1 hour, the aptamer enriched guide RNA library was desalted and purified using standard molecular biology techniques. Blocking the 3′ ends of the guide RNA library with dideoxy nucleotides prevents non-functional guides from being reisolated in subsequent steps. 10 picomoles of the TdT treated guide RNA library was incubated with Cas9 at an equimolar ratio at room temperature for 1 hour. A 68 nt DNA fragment containing the eGFP target sequence was annealed to its complement in 10 mM Tris-HCl pH 8.0 and 10 mM MgCl2 at 95° C. for 5 minutes and then snap-cooled on ice for 5 minutes. Both DNA fragments included a 5′ cyanine 5-aminoallyluridine-5′-triphosphate and a 3′ dideoxy cytosine (IDT). 1 picomole of the resulting double stranded fragment was incubated with 10 picomoles of TdT treated Cas9-library RNP complexes and incubated at 37° C. for 1 hour. The reaction was then supplemented with 0.1 mM biotin-16-aminoallyl-2′-dCTP (Trilink, N-5002), 100 units of TdT in the supplied buffers and incubated at 37° C. for 15 to 20 minutes. 1 ul of magnetic streptavidin beads (Thermofisher #65001) was added to the reaction and transferred to 4° C. for 2 hours with rotation. Beads were then washed in cleavage buffer 3 times and subjected to RNA purification steps, described above, to be prepared for subsequent rounds. Prior to purification, a portion of the sample was collected and subjected to flow cytometry to assess cleavage efficiency, guide RNA retention and background.

FIG. 7 shows that cleavage was observed in candidate mixtures having 58% and 73% degeneracy with the template guide nucleic acids after 5 rounds of binding affinity selection. The cleavage assay assessed the ability to cleave a 1700 base pair DNA fragment of GFP target DNA into smaller fragments of 1300 and 400 base pairs.

FIG. 8 shows that candidate sequences 254, 264, 258, and 243 were identified by the candidate mixture having 73% degeneracy to have cleavage activity. Although both the 58% and 73% degenerate candidate mixtures demonstrate cleavage activity, no individual sequences where identified from the 58% candidate mixtures that supported cleavage activity.

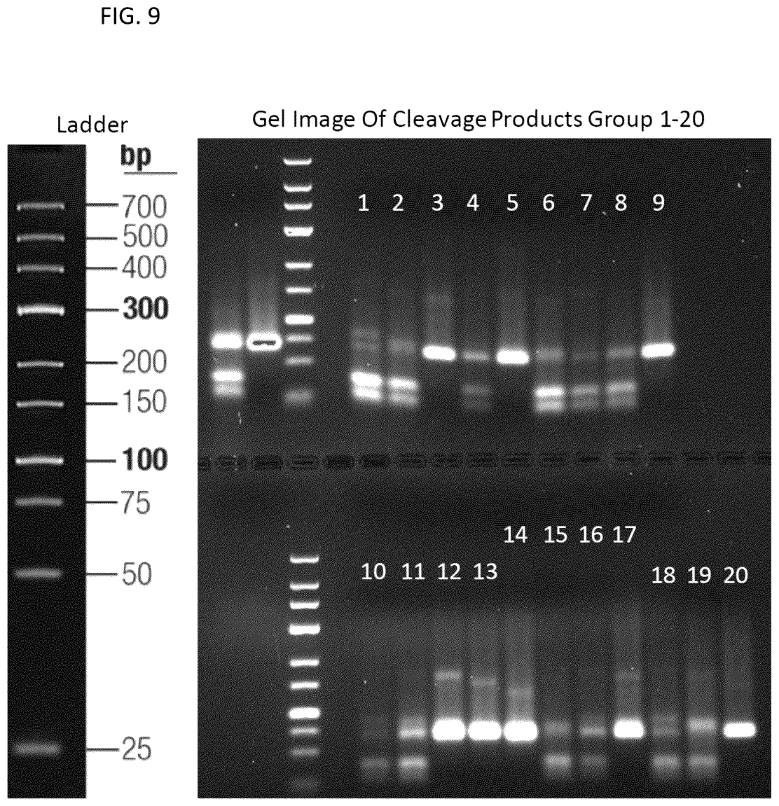

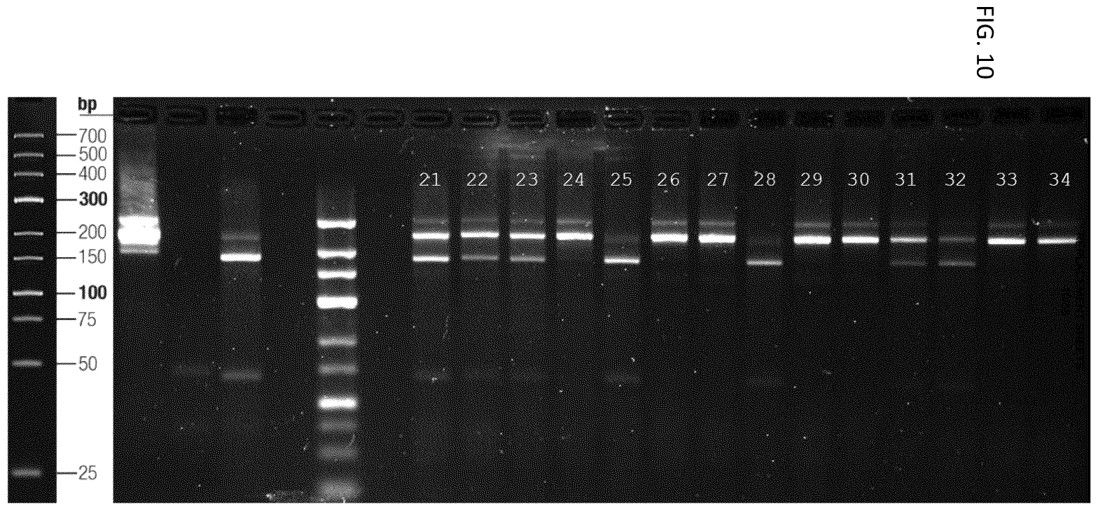

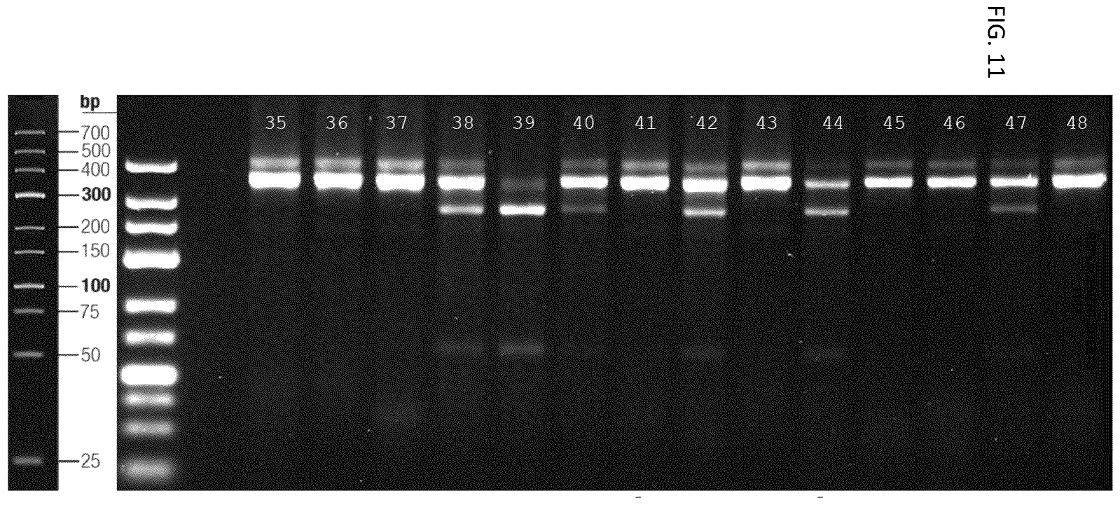

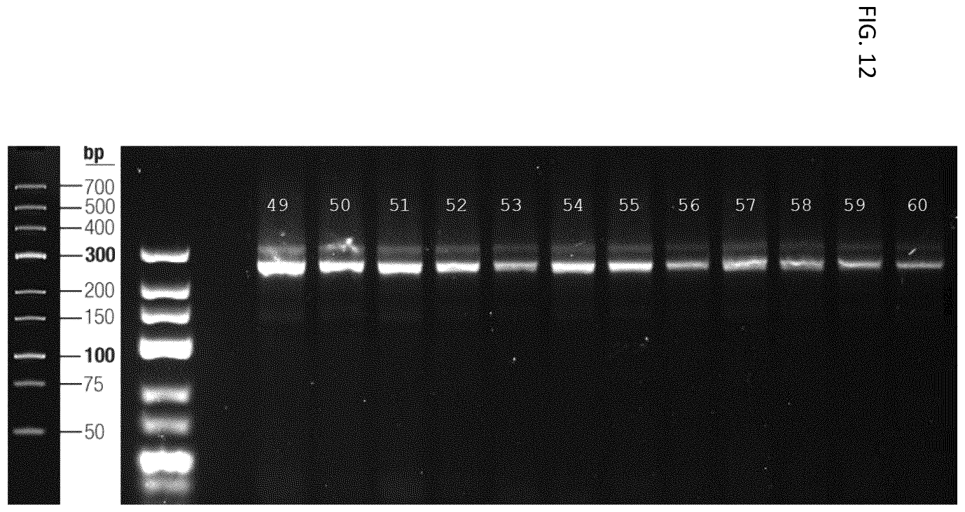

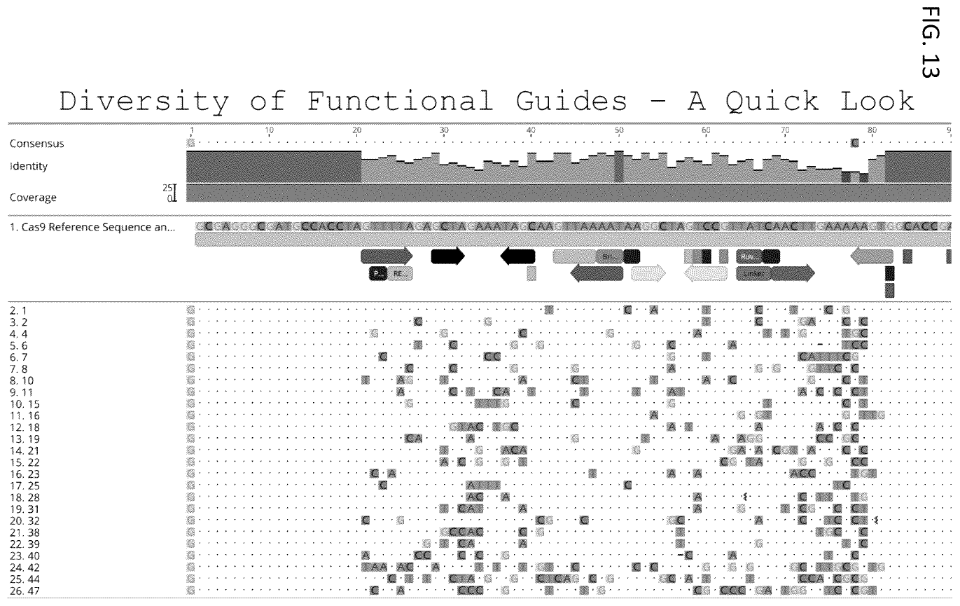

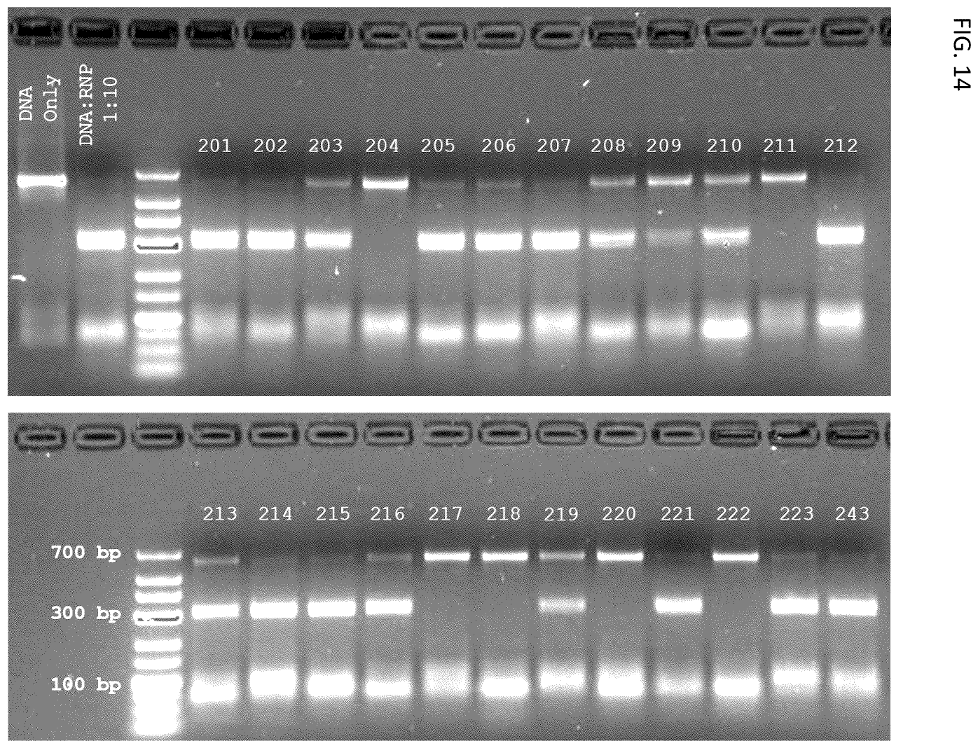

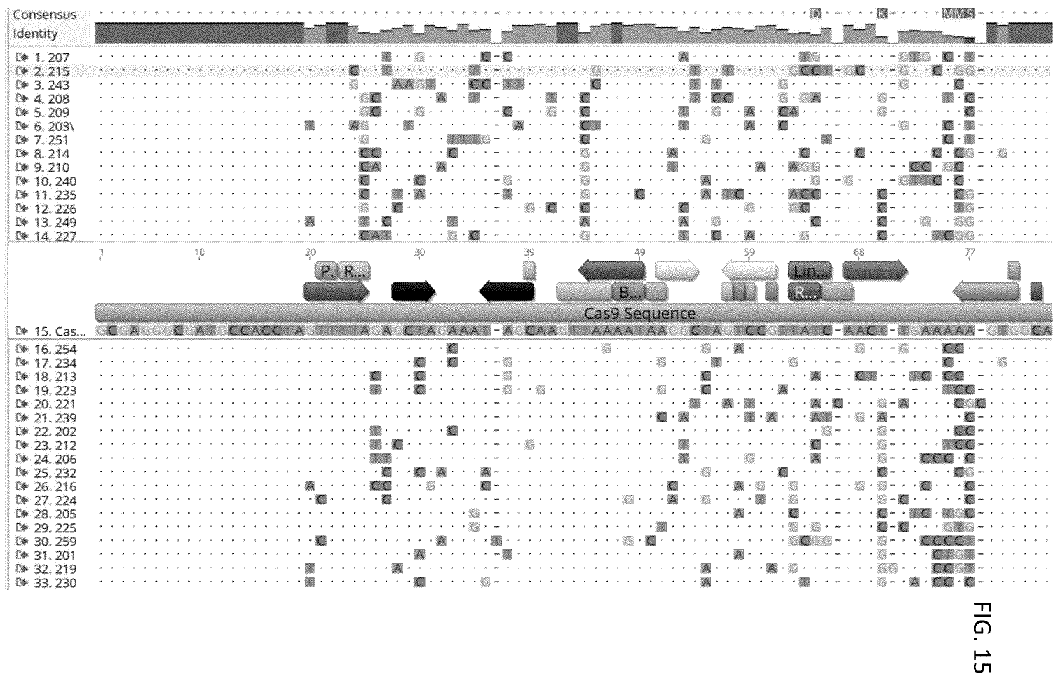

FIGS. 9-12 show candidate sequences ordered from high to low abundance partitioned after five rounds of binding affinity enrichment. The cleavage assay used a 2:1 ratio of DNA to RNP (FIG. 9) or 10:1 ratio of RNP to DNA (FIGS. 10-12) incubated overnight in a 100 mMNaCl cleavage buffer.

FIG. 13 shows candidate sequences supporting Cas9 cleavage activity identified from FIGS. 9-12.

FIG. 14 shows TDT selection enhanced selection of functional gRNA sequences in a cleavage assay comprised of NEB 3.1 buffer incubated for an hour with a ratio of 10:1 ratio of

RNP to DNA. The target was a 600 base pair fragment cleaved into 2 300 base pair fragments.

FIG. 15 shows candidate gRNA sequences that support cleavage activity identified from the experiment described in FIG. 14. As shown in the figure, the mutations tend to maintain secondary structure but the loops are highly variable.

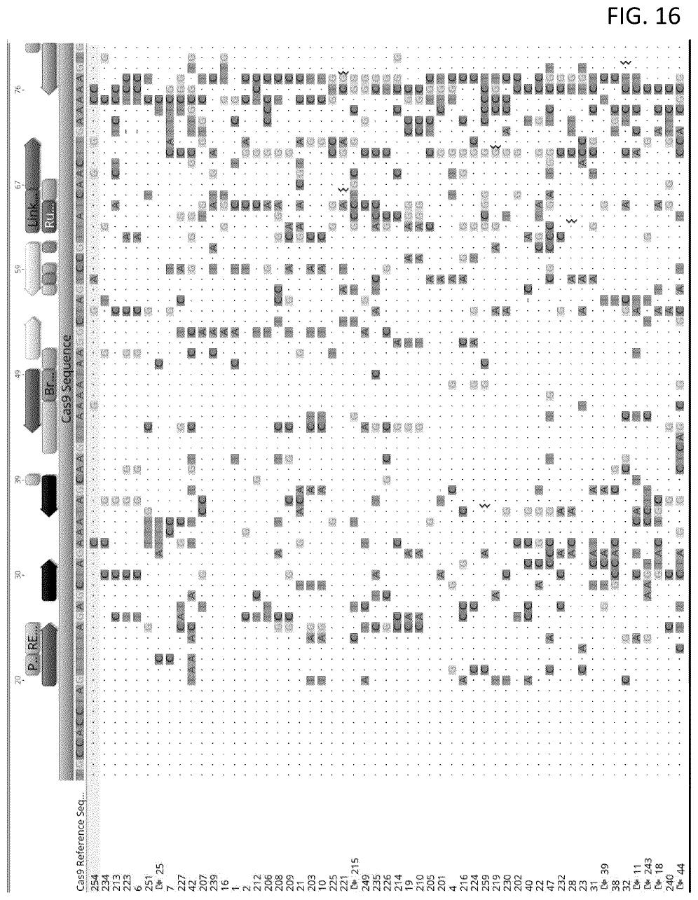

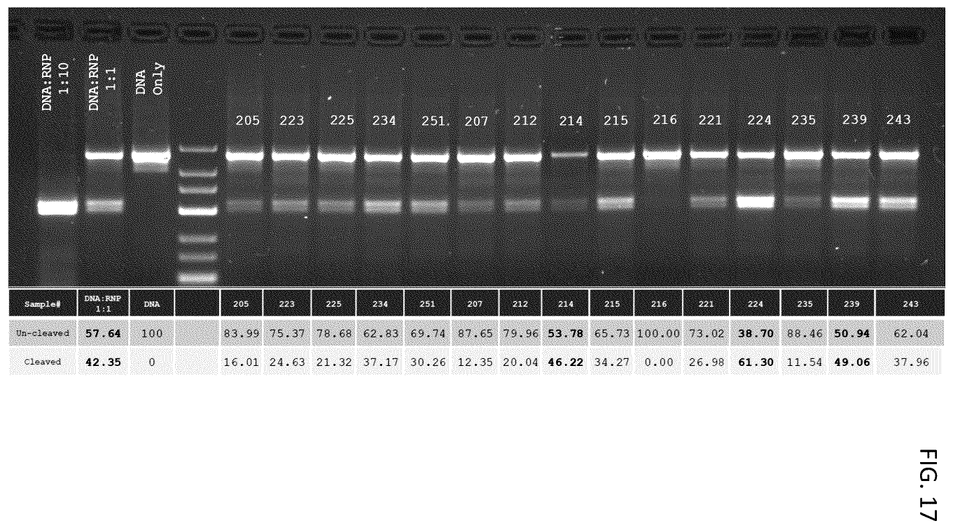

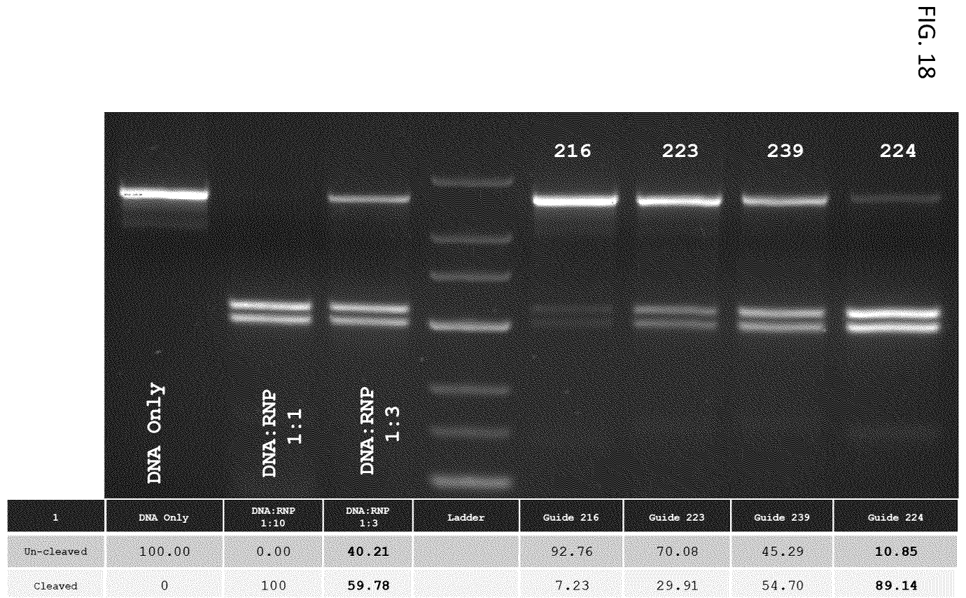

FIG. 16 shows candidate sequences supporting Cas9 cleavage activity identified from all of the experiments described herein.

FIGS. 17-18 shows the range of cleavage efficiency below the standard 10:1 RNP to DNA ratio.

In summary, the methods described herein were able to identify 55 novel and functional guide nucleotide sequences and many aptamer sequences that bind Cas9 but do not appear to support cleavage activity. Selection method #1, without cleavage activity partitioning, identified 23 and selection method #2, with cleavage activity partitioning, identified 32 additional sequences. Thirty three (33) of those guide nucleotide sequences displayed cleavage efficiency at least equivalent to the wildtype gRNA for the targeted DNA sequence. Those function guide nucleotides also demonstrated variation across the randomized scaffold. Taken together, these results demonstrate the ability to generate and identify novel functional guide nucleotides. See Table 1 below.

### Utilization of Guide RNA Variation to Enhance Cas9 Cleavage at Poorly Edited Sites

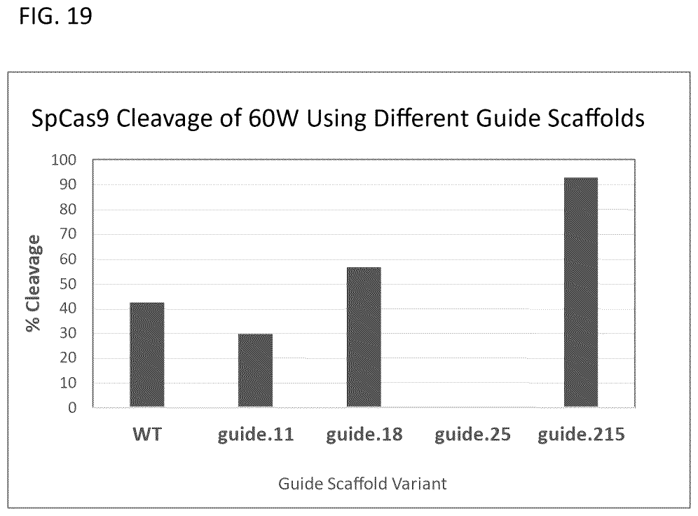

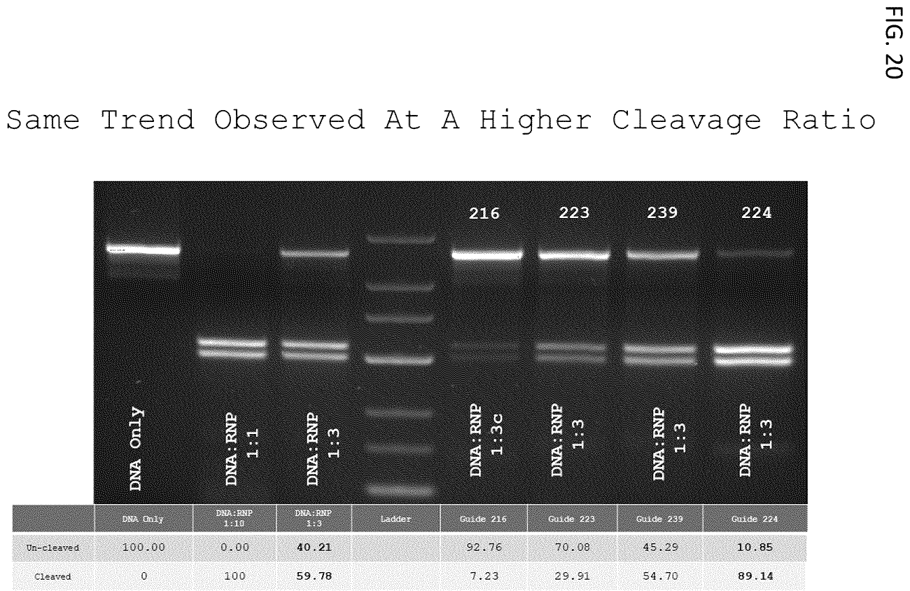

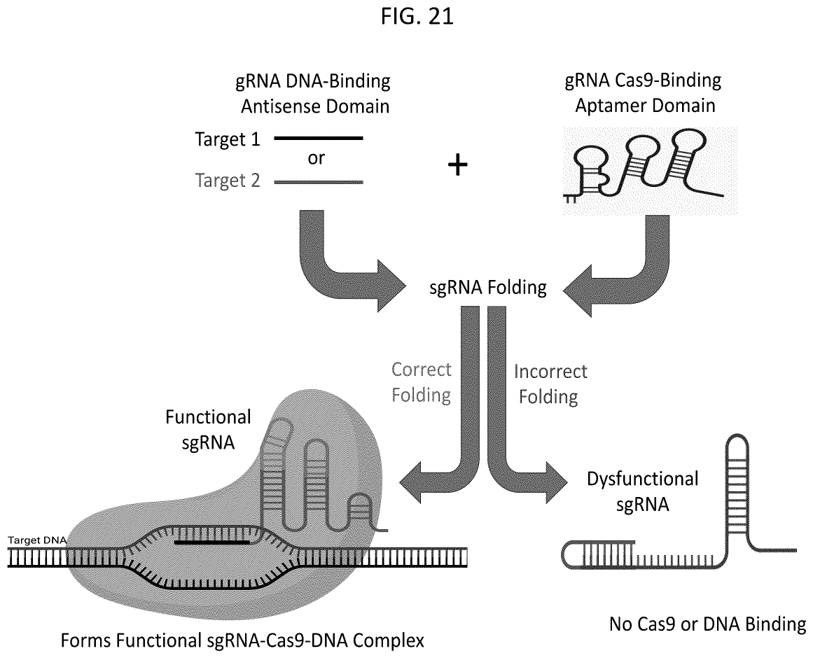

The inventors had tested a number of wild type guide RNAs targeted against the coagulation factor XII gene, a coagulation factor that is part of the contact pathway. In FIG. 19, each number represents a variant guide RNA as shown in the Table 1 below. All guides shown target the same location within the factor XII gene, known as 60 W. Many of the Cas9 RNPs had low cleavage efficiencies including the wild type scaffold with the targeting sequence directed to 60 FW. The wild type scaffold, as shown in the figure, has sub-optimal cleavage efficiency (<60%). The wild type scaffold was switched out with the variant guides to determine if switching the scaffold to one of our novel ones would enhance cleavage efficiency. Various sites within the factor XII gene were targeted with an array of variant guides. It was shown that guide 215, when targeted to the same region that yielded relatively low activity with the wild type scaffold, enhanced cleavage activity by about 40 percent.

Additionally, FIG. 20 shows that cleavage efficiency of the GFP gene could be enhanced In vitro when utilizing a variant guide. Based on the cleavage activity, it appears that variant 224 has a higher cleavage efficiency than the wild type at a 1 to 3 ratio. The bottom rows show percent cleavage efficiency. Taken together, the inventors believe that the set of novel functional guides acquired from our selection approach can be used to enhance Cas-cleavage activity when the scaffold interacts with the targeting region or when other less favorable interactions occur within the sgRNA.

TABLE 1
Exemplary nucleic acids.

| | DNA sequence utilized | DNA | | RNA | | |
|---|---|---|---|---|---|---|
| | for guide RNA | SEQ | | SEQ | | |
| | generation-Organized | ID | RNA of guide RNA-Organized | ID | | Sequence |
| Name | 5 prime to 3 prime | NO: | 5 prime to 3 prime | NO: | Length | Function |
| 55418 | TAATACGACTCACTATAGGCGAGGGCG | 9 | GCGAGGGCGAUGCCACCUAAUUUUAG | 10 | 95 | Functional |
| | ATGCCACCTAATTTTAGTGCTTGTAATA | | UGCUUGUAAUAGCAAGUUGAAAUAA | | | Sequences |
| | GCAAGTTGAAATAAGGCTAGTCCGTGA | | GGCUAGUCCGUGAACCACCUGAAACG | | | |
| | ACCACCTGAAACGGGGGCACCGAGTCG | | GGGGCACCGAGUCGGUGC | | | |
| | GTGC | | | | | |
| 52613 | TAATACGACTCACTATAGGCGAGGGCG | 11 | GCGAGGGCGAUGCCACCUAGUUUUAG | 12 | 95 | Functional |
| | ATGCCACCTAGTTTTAGAGCGCTGAAGC | | AGCGCUGAAGCGUCAGUUAAAAUAAA | | | Sequences |
| | GTCAGTTAAAATAAAGCTAGTCCGTTCA | | GCUAGUCCGUUCACAACUUGGCAUAG | | | |
| | CAACTTGGCATAGTGGCACCGAGTCGG | | UGGCACCGAGUCGGUGC | | | |
| | TGC | | | | | |
| 47700 | TAATACGACTCACTATAGGCGAGGGCG | 13 | GCGAGGGCGAUGCCACCUAAUUUUAU | 14 | 95 | Functional |
| | ATGCCACCTAATTTTATAGTTAGAGACA | | AGUUAGAGACAACAAGUUAAAAUAA | | | Sequences |
| | ACAAGTTAAAATAAGGCTAGTCCGTTA | | GGCUAGUCCGUUACCAACGUGAACAU | | | |
| | CCAACGTGAACATGGGGCACCGAGTCG | | GGGGCACCGAGUCGGUGC | | | |
| | GTGC | | | | | |
| 39550 | TAATACGACTCACTATAGGCGAGGGCG | 15 | GCGAGGGCGAUGCCACCUAGUUUUAG | 16 | 95 | Functional |
| | ATGCCACCTAGTTTTAGAGCTGGAAAA | | AGCUGGAAAAGGCAAGUUAAAAAAG | | | Sequences |
| | GGCAAGTTAAAAAAGGGCTAGTCCGCA | | GGCUAGUCCGCAAUCAACAUGAAAAC | | | |
| | ATCAACATGAAAACGTGGCACCGAGTC | | GUGGCACCGAGUCGGUGC | | | |
| | GGTGC | | | | | |
| 33818 | TAATACGACTCACTATAGGCGAGGGCG | 17 | GCGAGGGCGAUGCCACCUAGUUUUAG | 18 | 95 | Functional |
| | ATGCCACCTAGTTTTAGGTCTAGAAATA | | GUCUAGAAAUAGCGAGUUAAAAUAA | | | Sequences |
| | GCGAGTTAAAATAAGGACACTCCGTAC | | GGACACUCCGUACGCAACGGCAAAAC | | | |
| | GCAACGGCAAAACGTGGCACCGAGTCG | | GUGGCACCGAGUCGGUGC | | | |
| | GTGC | | | | | |
| 33635 | TAATACGACTCACTATAGGCGAGGGCG | 19 | GCGAGGGCGAUGCCACCUAGUUUAAU | 20 | 95 | Functional |
| | ATGCCACCTAGTTTAATAGCGAGTAATC | | AGCGAGUAAUCGCAUGUUUAAAUAA | | | Sequences |
| | GCATGTTTAAATAAGGCTAGACCGGTA | | GGCUAGACCGGUAACAAAUUGAAUCA | | | |
| | ACAAATTGAATCAGTGGCACCGAGTCG | | GUGGCACCGAGUCGGUGC | | | |
| | GTGC | | | | | |
| 30609 | TAATACGACTCACTATAGGCGAGGGCG | 21 | GCGAGGGCGAUGCCACCUAGUUUUAG | 22 | 95 | Functional |
| | ATGCCACCTAGTTTTAGAGCCAAAAAT | | AGCCAAAAAUGGCCAGUUAAAAUACG | | | Sequences |
| | GGCCAGTTAAAATACGGCAAGTCCATT | | GCAAGUCCAUUAGCAACAUGCACACG | | | |
| | AGCAACATGCACACGTGGCACCGAGTC | | UGGCACCGAGUCGGUGC | | | |
| | GGTGC | | | | | |
| 29744 | TAATACGACTCACTATAGGCGAGGGCG | 23 | GCGAGGGCGAUGCCACCUAGUGUUAG | 24 | 95 | Functional |
| | ATGCCACCTAGTGTTAGAGCTAGAAAT | | AGCUAGAAAUAGCAAGUUAACGUAA | | | Sequences |
| | AGCAAGTTAACGTAAGGCTAGTCCGCT | | GGCUAGUCCGCUAACAACCUGCAACG | | | |
| | AACAACCTGCAACGGTGGCACCGAGTC | | GUGGCACCGAGUCGGUGC | | | |
| | GGTGC | | | | | |
| 26084 | TAATACGACTCACTATAGGCGAGGGCG | 25 | GCGAGGGCGAUGCCACCUAGUUUUAG | 26 | 95 | Functional |
| | ATGCCACCTAGTTTTAGAGCTAGAAATA | | AGCUAGAAAUAGCAAGUUAAAAUCA | | | Sequences |
| | GCAAGTTAAAATCAGGCTAGTCCAAGA | | GGCUAGUCCAAGAACAACAUCAACAC | | | |
| | ACAACATCAACACGTGGCACCGAGTCG | | GUGGCACCGAGUCGGUGC | | | |
| | GTGC | | | | | |
| 25653 | TAATACGACTCACTATAGGCGAGGGCG | 27 | GCGAGGGCGAUGCCACCUAGAUUUAG | 28 | 95 | Functional |
| | ATGCCACCTAGATTTAGAGCTGGAAAC | | AGCUGGAAACAGCAAGUUAAAAUAA | | | Sequences |
| | AGCAAGTTAAAATAAGGCTTGTCCGTC | | GGCUUGUCCGUCAACAACUUGAAAAC | | | |
| | AACAACTTGAAAACGTGGCACCGAGTC | | GUGGCACCGAGUCGGUGC | | | |
| | GGTGC | | | | | |
| 25642 | TAATACGACTCACTATAGGCGAGGGCG | 29 | GCGAGGGCGAUGCCACCUAGUUUUAG | 30 | 95 | Functional |
| | ATGCCACCTAGTTTTAGAGCTAGCAATC | | AGCUAGCAAUCGCAAGUUAAAAUAAG | | | Sequences |
| | GCAAGTTAAAATAAGGATCGTCCGTTA | | GAUCGUCCGUUAUCAACUUGAAAGAG | | | |
| | TCAACTTGAAAGAGTGGCACCGAGTCG | | UGGCACCGAGUCGGUGC | | | |
| | GTGC | | | | | |
| 23661 | TAATACGACTCACTATAGGCGAGGGCG | 31 | GCGAGGGCGAUGCCACCUAGUUUUAG | 32 | 95 | Functional |
| | ATGCCACCTAGTTTTAGCGCAGAAAACT | | CGCAGAAAACUGUAAGUUAAAAUAA | | | Sequences |
| | GTAAGTTAAAATAAGGCTAGATCGTTA | | GGCUAGAUCGUUAACAACUGGAAUCA | | | |
| | ACAACTGGAATCAGTGGCACCGAGTCG | | GUGGCACCGAGUCGGUGC | | | |
| | GTGC | | | | | |
| 19753 | TAATACGACTCACTATAGGCGAGGGCG | 33 | GCGAGGGCGAUGCCACCUAGUUUUAG | 34 | 95 | Functional |
| | ATGCCACCTAGTTTTAGAGCTAGAAATA | | AGCUAGAAAUAGCAAGUUAAAAUAA | | | Sequences |
| | GCAAGTTAAAATAAGACGAGATCGATA | | GACGAGAUCGAUACCAACUUGAGAAU | | | |
| | CCAACTTGAGAATGTGGCACCGAGTCG | | GUGGCACCGAGUCGGUGC | | | |
| | GTGC | | | | | |
| 19406 | TAATACGACTCACTATAGGCGAGGGCG | 35 | GCGAGGGCGAUGCCACCUAGUAUUCG | 36 | 95 | Functional |
| | ATGCCACCTAGTATTCGAGTCAGAAAT | | AGUCAGAAAUGGCACGUGAAUAUAA | | | Sequences |
| | GGCACGTGAATATAAGACTAGTTCGTA | | GACUAGUUCGUACUCAACUGGCAAGC | | | |
| | CTCAACTGGCAAGCGTGGCACCGAGTC | | GUGGCACCGAGUCGGUGC | | | |
| | GGTGC | | | | | |
| 18301 | TAATACGACTCACTATAGGCGAGGGCG | 37 | GCGAGGGCGAUGCCACCUAGUUUUCG | 38 | 95 | Functional |
| | ATGCCACCTAGTTTTCGCAGTAGCAATA | | CAGUAGCAAUACCAAGUGAAAAUAAG | | | Sequences |
| | CCAAGTGAAAATAAGATTAGTCCGAAA | | AUUAGUCCGAAAUCAACGUGAAACCG | | | |
| | TCAACGTGAAACCGTGGCACCGAGTCG | | UGGCACCGAGUCGGUGC | | | |
| | GTGC | | | | | |
| 15985 | TAATACGACTCACTATAGGCGAGGGCG | 39 | GCGAGGGCGAUGCCACCUAGUUUUAG | 40 | 95 | Functional |
| | ATGCCACCTAGTTTTAGTGCTAGAAATG | | UGCUAGAAAUGGCAAGUUAAAAUAA | | | Sequences |
| | GCAAGTTAAAATAAGACCAGTTCGTTA | | GACCAGUUCGUUAUCUACCUGAGUGC | | | |
| | TCTACCTGAGTGCGTGGCACCGAGTCG | | GUGGCACCGAGUCGGUGC | | | |
| | GTGC | | | | | |
| 14148 | TAATACGACTCACTATAGGCGAGGGCG | 41 | GCGAGGGCGAUGCCACCUAGUUUUAG | 42 | 95 | Functional |
| | ATGCCACCTAGTTTTAGTGCGAGAATTC | | UGCGAGAAUUCGCAAGUUAAAAUCAG | | | Sequences |
| | GCAAGTTAAAATCAGTCAAATACGTTG | | UCAAAUACGUUGUCACCGUGCAAUCG | | | |
| | TCACCGTGCAATCGTGGCACCGAGTCG | | UGGCACCGAGUCGGUGC | | | |
| | GTGC | | | | | |
| 11187 | TAATACGACTCACTATAGGCGAGGGCG | 43 | GCGAGGGCGAUGCCACCUAGUUUUAG | 44 | 95 | Functional |
| | ATGCCACCTAGTTTTAGCGCTTGAAAAA | | CGCUUGAAAAAGCAAGUUAAAAUAA | | | Sequences |
| | GCAAGTTAAAATAAGGCTAGTCCGTTA | | GGCUAGUCCGUUAGUUAACGGAACAU | | | |
| | GTTAACGGAACATGTGGCACCGAGTCG | | GUGGCACCGAGUCGGUGC | | | |
| | GTGC | | | | | |
| 8221 | TAATACGACTCACTATAGGCGAGGGCG | 45 | GCGAGGGCGAUGCCACCUAGUUUUAG | 46 | 95 | Functional |
| | ATGCCACCTAGTTTTAGAGCGGGAAAA | | AGCGGGAAAACGCAUGUUAAAACAAG | | | Sequences |
| | CGCATGTTAAAACAAGACTAGTCCGTT | | ACUAGUCCGUUACCACCGUUAAACCG | | | |
| | ACCACCGTTAAACCGTGGCACCGAGTC | | UGGCACCGAGUCGGUGC | | | |
| | GGTGC | | | | | |
| 4698 | TAATACGACTCACTATAGGCGAGGGCG | 47 | GCGAGGGCGAUGCCACCUAAUUUUCU | 48 | 95 | Functional |
| | ATGCCACCTAATTTTCTAGCTAGCAATA | | AGCUAGCAAUAGCAUGUGAAAAUAA | | | Sequences |
| | GCATGTGAAAATAAGGCTAGACCGATG | | GGCUAGACCGAUGUCAACUUGUUCGG | | | |
| | TCAACTTGTTCGGGTGGCACCGAGTCGG | | GUGGCACCGAGUCGGUGC | | | |
| | TGC | | | | | |
| 2255 | TAATACGACTCACTATAGGCGAGGGCG | 49 | GCGAGGGCGAUGCCACCUAGUUUCAC | 50 | 95 | Functional |
| | ATGCCACCTAGTTTCACAGCGCGAAATC | | AGCGCGAAAUCGCAAGUUGAAAUAAG | | | Sequences |
| | GCAAGTTGAAATAAGACTAGTTCGGTA | | ACUAGUUCGGUAGCAACAUGACAAUG | | | |
| | GCAACATGACAATGTGGCACCGAGTCG | | UGGCACCGAGUCGGUGC | | | |
| | GTGC | | | | | |
| 2037 | TAATACGACTCACTATAGGCGAGGGCG | 51 | GCGAGGGCGAUGCCACCUAGUUUUAG | 52 | 95 | Functional |
| | ATGCCACCTAGTTTTAGTGCTCGAAAGA | | UGCUCGAAAGAGAAAGUUAAAAUAA | | | Sequences |
| | GAAAGTTAAAATAAGAACATTTCGCGA | | GAACAUUUCGCGAUCACCGUUAAUAC | | | |
| | TCACCGTTAATACGTGGCACCGAGTCG | | GUGGCACCGAGUCGGUGC | | | |
| | GTGC | | | | | |
| 1522 | TAATACGACTCACTATAGGCGAGGGCG | 53 | GCGAGGGCGAUGCCACCUAGUUUUAU | 54 | 95 | Functional |
| | ATGCCACCTAGTTTTATCGGTAGAAAAA | | CGGUAGAAAAACCAUGUUAAAAUAU | | | Sequences |
| | CCATGTTAAAATATGGCTAGTCCGGTGA | | GGCUAGUCCGGUGACAACGGGAUGCC | | | |
| | CAACGGGATGCCGTGGCACCGAGTCGG | | GUGGCACCGAGUCGGUGC | | | |
| | TGC | | | | | |
| 1414 | TAATACGACTCACTATAGGCGAGGGCG | 55 | GCGAGGGCGAUGCCACCUAGUUUGAG | 56 | 95 | Functional |
| | ATGCCACCTAGTTTGAGAACTAGAAAT | | AACUAGAAAUAGAAAGUUCAAAUAA | | | Sequences |
| | AGAAAGTTCAAATAAGGTTAATCCGTT | | GGUUAAUCCGUUAUCAACUUGAAACA | | | |
| | ATCAACTTGAAACAGTGGCACCGAGTC | | GUGGCACCGAGUCGGUGC | | | |
| | GGTGC | | | | | |
| 580 | TAATACGACTCACTATAGGCGAGGGCG | 57 | GCGAGGGCGAUGCCACCUAGUUUUCG | 58 | 95 | Functional |
| | ATGCCACCTAGTTTTCGCGCCAGAAACG | | CGCCAGAAACGGCAAGUGAAAAUAAG | | | Sequences |
| | GCAAGTGAAAATAAGACTAGTTCGTAA | | ACUAGUUCGUAAACCACUGGAAACGG | | | |
| | ACCACTGGAAACGGTGGCACCGAGTCG | | UGGCACCGAGUCGGUGC | | | |
| | GTGC | | | | | |
| 564 | TAATACGACTCACTATAGGCGAGGGCG | 59 | GCGAGGGCGAUGCCACCUAGUUUGAG | 60 | 95 | Functional |
| | ATGCCACCTAGTTTGAGTGCTAGTAATA | | UGCUAGUAAUAGCAAGUUCAAAUAA | | | Sequences |
| | GCAAGTTCAAATAAGGATAGACCGCAA | | GGAUAGACCGCAAACACCGUGAACAG | | | |
| | ACACCGTGAACAGGTGGCACCGAGTCG | | GUGGCACCGAGUCGGUGC | | | |
| | GTGC | | | | | |
| 312 | TAATACGACTCACTATAGGCGAGGGCG | 61 | GCGAGGGCGAUGCCACCUAGUUUUAG | 62 | 95 | Functional |
| | ATGCCACCTAGTTTTAGAGCGCGAAATC | | AGCGCGAAAUCGCAAGUUAAAAUAAG | | | Sequences |
| | GCAAGTTAAAATAAGACTAGTGCGTTC | | ACUAGUGCGUUCACAACUUCAGCAAG | | | |
| | ACAACTTCAGCAAGTGGCACCGAGTCG | | UGGCACCGAGUCGGUGC | | | |
| | GTGC | | | | | |
| 1 | TAATACGACTCACTATAGGCGAGGGCG | 63 | GCGAGGGCGAUGCCACCUAGUUUUAG | 64 | 95 | Functional |
| | ATGCCACCTAGTTTTAGAGCTAGAAATA | | AGCUAGAAAUAGCAUGUUAAAAUCA | | | Sequences |
| | GCATGTTAAAATCAGACTAGTTCGTTAC | | GACUAGUUCGUUACCAAUUUGCAGAA | | | |
| | CAATTTGCAGAAGTGGCACCGAGTCGG | | GUGGCACCGAGUCGGUGC | | | |
| | TGC | | | | | |
| 2 | TAATACGACTCACTATAGGCGAGGGCG | 65 | GCGAGGGCGAUGCCACCUAGUUUUAC | 66 | 95 | Functional |
| | ATGCCACCTAGTTTTACAGCTAGAGATA | | AGCUAGAGAUAGCAAGUUAAAAUAA | | | Sequences |
| | GCAAGTTAAAATAAGGCTAGTTCGTTA | | GGCUAGUUCGUUACCAACGAGAACAC | | | |
| | CCAACGAGAACACGTGGCACCGAGTCG | | GUGGCACCGAGUCGGUGC | | | |
| | GTGC | | | | | |
| 4 | TAATACGACTCACTATAGGCGAGGGCG | 67 | GCGAGGGCGAUGCCACCUAGGUUUAG | 68 | 95 | Functional |
| | ATGCCACCTAGGTTTAGAGGTAGAAAT | | AGGUAGAAAUACCAAGUUAAAGUAA | | | Sequences |
| | ACCAAGTTAAAGTAAGGCTAGACCGTT | | GGCUAGACCGUUAUUAUCGUGAAUGC | | | |
| | ATTATCGTGAATGCGTGGCACCGAGTC | | GUGGCACCGAGUCGGUGC | | | |
| | GGTGC | | | | | |
| 6 | TAATACGACTCACTATAGGCGAGGGCG | 69 | GCGAGGGCGAUGCCACCUAGUUUUAU | 70 | 95 | Functional |
| | ATGCCACCTAGTTTTATAGCCAGAAATG | | AGCCAGAAAUGGCGAGUUAAAAUAG | | | Sequences |
| | GCGAGTTAAAATAGGGCCAGTCCGATA | | GGCCAGUCCGAUAUCAACUUAAUCCG | | | |
| | TCAACTTAATCCGTGGCACCGAGTCGGT | | UGGCACCGAGUCGGUGC | | | |
| | GC | | | | | |
| 7 | TAATACGACTCACTATAGGCGAGGGCG | 71 | GCGAGGGCGAUGCCACCUAGUCUUAG | 72 | 95 | Functional |
| | ATGCCACCTAGTCTTAGAGCTAGACCTA | | AGCUAGACCUAGCAAGUUAAAAUAAG | | | Sequences |
| | GCAAGTTAAAATAAGGCGAGTTCGTTA | | GCGAGUUCGUUAUCAACCAUUUCGAG | | | |
| | TCAACCATTTCGAGTGGCACCGAGTCG | | UGGCACCGAGUCGGUGC | | | |
| | GTGC | | | | | |
| 10 | TAATACGACTCACTATAGGCGAGGGCG | 73 | GCGAGGGCGAUGCCACCUAUUUUAGG | 74 | 94 | Functional |
| | ATGCCACCTATTTTAGGAGTTAGAAATA | | AGUUAGAAAUAACAAGUCUAAAUAA | | | Sequences |
| | ACAAGTCTAAATAAGTCTAGTACGCTAT | | GUCUAGUACGCUAUCAACUGGAACAU | | | |
| | CAACTGGAACATGTGGCACCGAGTCGG | | GUGGCACCGAGUCGGUGC | | | |
| | TGC | | | | | |
| 11 | TAATACGACTCACTATAGGCGAGGGCG | 75 | GCGAGGGCGAUGCCACCUAGUUUAAG | 76 | 95 | Functional |
| | ATGCCACCTAGTTTAAGAGCCATAACA | | AGCCAUAACAAGUAAGUUUAAAUAU | | | Sequences |
| | AGTAAGTTTAAATATGGCATGTCCGTTA | | GGCAUGUCCGUUAUCAACAUCACACU | | | |
| | TCAACATCACACTGTGGCACCGAGTCG | | GUGGCACCGAGUCGGUGC | | | |
| | GTGC | | | | | |
| 16 | TAATACGACTCACTATAGGCGAGGGCG | 77 | GCGAGGGCGAUGCCACCUAGUUUUAG | 78 | 95 | Functional |
| | ATGCCACCTAGTTTTAGAGCTAGAAATA | | AGCUAGAAAUAGCAAGUUAAAAUAA | | | Sequences |
| | GCAAGTTAAAATAAGACTAGTCCGTGA | | GACUAGUCCGUGAGUAACUUGAAGAU | | | |
| | GTAACTTGAAGATTGGGCACCGAGTCG | | UGGGCACCGAGUCGGUGC | | | |
| | GTGC | | | | | |
| 18 | TAATACGACTCACTATAGGCGAGGGCG | 79 | GCGAGGGCGAUGCCACCUAGUUUUAG | 80 | 95 | Functional |
| | ATGCCACCTAGTTTTAGAGCGTACATGC | | AGCGUACAUGCGCAAGUUAAAAUAAG | | | Sequences |
| | GCAAGTTAAAATAAGGCAATTCCGTTA | | GCAAUUCCGUUAACAACUUAACACAG | | | |
| | ACAACTTAACACAGTGGCACCGAGTCG | | UGGCACCGAGUCGGUGC | | | |
| | GTGC | | | | | |
| 19 | TAATACGACTCACTATAGGCGAGGGCG | 81 | GCGAGGGCGAUGCCACCUAGUUUUCA | 82 | 94 | Functional |
| | ATGCCACCTAGTTTTCAAGCTAAAAATA | | AGCUAAAAAUAGCAAGUGAAAAUAA | | | Sequences |
| | GCAAGTGAAAATAATGCTAGTCAGTAG | | UGCUAGUCAGUAGGCAACUUCCAGCA | | | |
| | GCAACTTCCAGCAGTGGCACCGAGTCG | | GUGGCACCGAGUCGGUGC | | | |
| | GTGC | | | | | |
| 21 | TAATACGACTCACTATAGGCGAGGGCG | 83 | GCGAGGGCGAUGCCACCUAGUUUUAG | 84 | 95 | Functional |
| | ATGCCACCTAGTTTTAGAGTTAGGAAAC | | AGUUAGGAAACACAAGUUAAAAUAG | | | Sequences |
| | ACAAGTTAAAATAGGGCTAGTCCGGAA | | GGCUAGUCCGGAAACCGUUAGAACAC | | | |
| | ACCGTTAGAACACGTGGCACCGAGTCG | | GUGGCACCGAGUCGGUGC | | | |
| | GTGC | | | | | |
| 22 | TAATACGACTCACTATAGGCGAGGGCG | 85 | GCGAGGGCGAUGCCACCUAGUUUUAG | 86 | 95 | Functional |
| | ATGCCACCTAGTTTTAGAGATCGGAAG | | AGAUCGGAAGAUCAAGUUAAAAUAA | | | Sequences |
| | ATCAAGTTAAAATAAGGCTAGTCCCGTT | | GGCUAGUCCCGUUACAACGUGGAACC | | | |
| | ACAACGTGGAACCGTGGCACCGAGTCG | | GUGGCACCGAGUCGGUGC | | | |
| | GTGC | | | | | |
| 23 | TAATACGACTCACTATAGGCGAGGGCG | 87 | GCGAGGGCGAUGCCACCUAGCUAUAG | 88 | 97 | Functional |
| | ATGCCACCTAGCTATAGAGCTAGAAAT | | AGCUAGAAAUAGCAAGUUAUAAUAA | | | Sequences |
| | AGCAAGTTATAATAAGGCAAGACCGTT | | GGCAAGACCGUUAUCAAACCGAAAUG | | | |
| | ATCAAACCGAAATGTTGGCACCGAGTC | | UUGGCACCGAGUCGGUGC | | | |
| | GGTGC | | | | | |
| 25 | TAATACGACTCACTATAGGCGAGGGCG | 89 | GCGAGGGCGAUGCCACCUAGUCUUAG | 90 | 95 | Functional |
| | ATGCCACCTAGTCTTAGAGCTAATTTTA | | AGCUAAUUUUAGCAAGUUAAAAUCA | | | Sequences |
| | GCAAGTTAAAATCAGGCTAGTCCGTTAT | | GGCUAGUCCGUUAUCAACUUGAUCAA | | | |
| | CAACTTGATCAAGTGGCACCGAGTCGG | | GUGGCACCGAGUCGGUGC | | | |
| | TGC | | | | | |
| 28 | TAATACGACTCACTATAGGCGAGGGCG | 91 | GCGAGGGCGAUGCCACCUAGUUUUAG | 92 | 95 | Functional |
| | ATGCCACCTAGTTTTAGAGCTAACAAA | | AGCUAACAAAAGCAAGUUAAAAUAA | | | Sequences |
| | AGCAAGTTAAAATAAGGCTAGACCGTT | | GGCUAGACCGUUUAUCAACCUUUAAU | | | |
| | TATCAACCTTTAATGGTGGCACCGAGTC | | GGUGGCACCGAGUCGGUGC | | | |
| | GGTGC | | | | | |
| 31 | TAATACGACTCACTATAGGCGAGGGCG | 93 | GCGAGGGCGAUGCCACCUAGUUUUAG | 94 | 95 | Functional |
| | ATGCCACCTAGTTTTAGAGTTCATAATA | | AGUUCAUAAUAACAAGUUAAAAUAA | | | Sequences |
| | ACAAGTTAAAATAAGGCTAGACCGTGA | | GGCUAGACCGUGAUCAUCCGGACACU | | | |
| | TCATCCGGACACTGTGGCACCGAGTCG | | GUGGCACCGAGUCGGUGC | | | |
| | GTGC | | | | | |
| 32 | TAATACGACTCACTATAGGCGAGGGCG | 95 | GCGAGGGCGAUGCCACCUACUUUGAG | 96 | 95 | Functional |
| | ATGCCACCTACTTTGAGAGCTAGAAAT | | AGCUAGAAAUAGCCGGUUCAAAUAAG | | | Sequences |
| | AGCCGGTTCAAATAAGGCGCGTCCGTT | | GCGCGUCCGUUAACAACCUGUCACUG | | | |
| | AACAACCTGTCACTGGTGGCACCGAGT | | GUGGCACCGAGUCGGUGC | | | |
| | CGGTGC | | | | | |
| 38 | TAATACGACTCACTATAGGCGAGGGCG | 97 | GCGAGGGCGAUGCCACCUAGUUUUAG | 98 | 95 | Functional |
| | ATGCCACCTAGTTTTAGAGGCCACAATA | | AGGCCACAAUACCGAGUUAAAAUAAG | | | Sequences |
| | CCGAGTTAAAATAAGGCTTGTCCGTTAT | | GCUUGUCCGUUAUCAACUUUGCAACG | | | |
| | CAACTTTGCAACGTGGCACCGAGTCGG | | UGGCACCGAGUCGGUGC | | | |
| | TGC | | | | | |
| 39 | TAATACGACTCACTATAGGCGAGGGCG | 99 | GCGAGGGCGAUGCCACCUAGUUUUAG | 100 | 95 | Functional |
| | ATGCCACCTAGTTTTAGGGTTCAAAATA | | GGUUCAAAAUAACAAGUUAAAAUAA | | | Sequences |
| | ACAAGTTAAAATAAGGCTTGTCCGTTA | | GGCUUGUCCGUUAGCAACUUGAAUAC | | | |
| | GCAACTTGAATACGTGGCACCGAGTCG | | GUGGCACCGAGUCGGUGC | | | |
| | GTGC | | | | | |
| 40 | TAATACGACTCACTATAGGCGAGGGCG | 101 | GCGAGGGCGAUGCCACCUAAUUUUAC | 102 | 95 | Functional |
| | ATGCCACCTAATTTTACCGCTCGCAAGA | | CGCUCGCAAGAGCAAGUUAAAAUAAG | | | Sequences |
| | GCAAGTTAAAATAAGGCTCTCCGATAT | | GCUCUCCGAUAUCAACUUGUAACAGU | | | |
| | CAACTTGTAACAGTGGCACCGAGTCGG | | GGCACCGAGUCGGUGC | | | |
| | TGC | | | | | |
| 42 | TAATACGACTCACTATAGGCGAGGGCG | 103 | GCGAGGGCGAUGCCACCUAUAAUACG | 104 | 95 | Functional |
| | ATGCCACCTATAATACGAACTAGTATTA | | AACUAGUAUUAUCGUGUCAAAAUACG | | | Sequences |
| | TCGTGTCAAAATACGCCTAGTGCGGTGT | | CCUAGUGCGGUGUCAAGCUUUGCGAU | | | |
| | CAAGCTTTGCGATGGGCACCGAGTCGG | | GGGCACCGAGUCGGUGC | | | |
| | TGC | | | | | |
| 44 | TAATACGACTCACTATAGGCGAGGGCG | 105 | GCGAGGGCGAUGCCACCUAGUUCUUG | 106 | 95 | Functional |
| | ATGCCACCTAGTTCTTGTGCCTAAGATG | | UGCCUAAGAUGGCCUCAGACAGUAAG | | | Sequences |
| | GCCTCAGACAGTAAGGGCAATTCGTTTT | | GGCAAUUCGUUUUCAACCCAACGCGG | | | |
| | CAACCCAACGCGGTGGCACCGAGTCGG | | UGGCACCGAGUCGGUGC | | | |
| | TGC | | | | | |
| 47 | TAATACGACTCACTATAGGCGAGGGCG | 107 | GCGAGGGCGAUGCCACCUAGCUUAAG | 108 | 95 | Functional |
| | ATGCCACCTAGCTTAAGAGCTCCCAAG | | AGCUCCCAAGAGCAUGUUUAGAUAAG | | | Sequences |
| | AGCATGTTTAGATAAGGCTAGCGCCCC | | GCUAGCGCCCCAGAAUGGUGUCACGU | | | |
| | AGAATGGTGTCACGTTGGCACCGAGTC | | UGGCACCGAGUCGGUGC | | | |
| | GGTGC | | | | | |
| 201 | TAATACGACTCACTATAGGCGAGGGCG | 109 | GCGAGGGCGAUGCCACCUAGUUUUAG | 110 | 95 | Functional |
| | ATGCCACCTAGTTTTAGAGCAAGAAATT | | AGCAAGAAAUUGCAAGUUAAAAUAA | | | Sequences |
| | GCAAGTTAAAATAAGGCTAGACCGTTA | | GGCUAGACCGUUAUCAACGUGACUGU | | | |
| | TCAACGTGACTGTGTGGCACCGAGTCG | | GUGGCACCGAGUCGGUGC | | | |
| | GTGC | | | | | |
| 202 | TAATACGACTCACTATAGGCGAGGGCG | 111 | GCGAGGGCGAUGCCACCUAGUUUUAU | 112 | 95 | Functional |
| | ATGCCACCTAGTTTTATAGCTAGCAATA | | AGCUAGCAAUAGCAAGUUAAAAUAA | | | Sequences |
| | GCAAGTTAAAATAAGGCTAGTCCGTTA | | GGCUAGUCCGUUAUGAACGUGAAACC | | | |
| | TGAACGTGAAACCGTGGCACCGAGTCG | | GUGGCACCGAGUCGGUGC | | | |
| | GTGC | | | | | |
| 203 | TAATACGACTCACTATAGGCGAGGGCG | 113 | GCGAGGGCGAUGCCACCUAUUUUAGG | 114 | 96 | Functional |
| | ATGCCACCTATTTTAGGAGTTAGAAATA | | AGUUAGAAAUAACAAGUCUAAAUAA | | | Sequences |
| | ACAAGTCTAAATAAGTCTAGTACGCTAT | | GUCUAGUACGCUAUCAACUGGAACAU | | | |
| | CAACTGGAACATGTGGCACCGAGTCGG | | GUGGCACCGAGUCGGUGC | | | |
| | TGC | | | | | |
| 205 | TAATACGACTCACTATAGGCGAGGGCG | 115 | GCGAGGGCGAUGCCACCUAGUUUUAG | 116 | 95 | Functional |
| | ATGCCACCTAGTTTTAGAGCTAGAAGTA | | AGCUAGAAGUAGCAAGUUAAAAUAA | | | Sequences |
| | GCAAGTTAAAATAAGGCTAGACCGTCA | | GGCUAGACCGUCAUCAACCUUCAUGC | | | |
| | TCAACCTTCATGCGTGGCACCGAGTCGG | | GUGGCACCGAGUCGGUGC | | | |
| | TGC | | | | | |
| 206 | TAATACGACTCACTATAGGCGAGGGCG | 117 | GCGAGGGCGAUGCCACCUAGUUUUAU | 118 | 96 | Functional |
| | ATGCCACCTAGTTTTATTGCTAGAAATA | | UGCUAGAAAUAGCAAGUUAAAAUAA | | | Sequences |
| | GCAAGTTAAAATAAGTCTAGTGCGTTA | | GUCUAGUGCGUUAACAACGUGCCCAC | | | |
| | ACAACGTGCCCACGTGGCACCGAGTCG | | GUGGCACCGAGUCGGUGC | | | |
| | GTGC | | | | | |
| 207 | TAATACGACTCACTATAGGCGAGGGCG | 119 | GCGAGGGCGAUGCCACCUAGUUUUAG | 120 | 96 | Functional |
| | ATGCCACCTAGTTTTAGTGCGAGAAACC | | UGCGAGAAACCGCAAGUUAAAAUAAG | | | Sequences |
| | GCAAGTTAAAATAAGACTAGTCCGTTT | | ACUAGUCCGUUUGCAACUGUGACAUG | | | |
| | GCAACTGTGACATGTGGCACCGAGTCG | | UGGCACCGAGUCGGUGC | | | |
| | GTGC | | | | | |
| 208 | TAATACGACTCACTATAGGCGAGGGCG | 121 | GCGAGGGCGAUGCCACCUAGUUUUGC | 122 | 95 | Functional |
| | ATGCCACCTAGTTTTGCAGCTAAAATTA | | AGCUAAAAUUAGCAUGUCAAAAUAA | | | Sequences |
| | GCATGTCAAAATAAGGTTCCTCCGGTG | | GGUUCCUCCGGUGACAACGUGAAUAC | | | |
| | ACAACGTGAATACGTGGCACCGAGTCG | | GUGGCACCGAGUCGGUGC | | | |
| | GTGC | | | | | |
| 209 | TAATACGACTCACTATAGGCGAGGGCG | 123 | GCGAGGGCGAUGCCACCUAGUUUUGC | 124 | 95 | Functional |
| | ATGCCACCTAGTTTTGCAGCGAGAAATC | | AGCGAGAAAUCGCAGGUCAAAAUAAG | | | Sequences |
| | GCAGGTCAAAATAAGTCTGGTACGCAA | | UCUGGUACGCAAUCAACGUGAAAACG | | | |
| | TCAACGTGAAAACGTGGCACCGAGTCG | | UGGCACCGAGUCGGUGC | | | |
| | GTGC | | | | | |
| 210 | TAATACGACTCACTATAGGCGAGGGCG | 125 | GCGAGGGCGAUGCCACCUAGUUUUCA | 126 | 95 | Functional |
| | ATGCCACCTAGTTTTCAAGCTAAAAATA | | AGCUAAAAAUAGCAAGUGAAAAUAA | | | Sequences |
| | GCAAGTGAAAATAATGCTAGTCAGTAG | | UGCUAGUCAGUAGGCAACUUCCAGCA | | | |
| | GCAACTTCCAGCAGTGGCACCGAGTCG | | GUGGCACCGAGUCGGUGC | | | |
| | GTGC | | | | | |
| 212 | TAATACGACTCACTATAGGCGAGGGCG | 127 | GCGAGGGCGAUGCCACCUAGUUUUAU | 128 | 95 | Functional |
| | ATGCCACCTAGTTTTATACCTAGAAATA | | ACCUAGAAAUAGGAAGUUAAAAUAA | | | Sequences |
| | GGAAGTTAAAATAAGTCTAGTCCGTTA | | GUCUAGUCCGUUACCAACGUGAAUCC | | | |
| | CCAACGTGAATCCGTGGCACCGAGTCG | | GUGGCACCGAGUCGGUGC | | | |
| | GTGC | | | | | |
| 213 | TAATACGACTCACTATAGGCGAGGGCG | 129 | GCGAGGGCGAUGCCACCUAGUUUUAC | 130 | 95 | Functional |
| | ATGCCACCTAGTTTTACAGCCAGAAATG | | AGCCAGAAAUGGCAAGUUAAAAUAA | | | Sequences |
| | GCAAGTTAAAATAAGGCCAGTCCGTTA | | GGCCAGUCCGUUAACACUUUUCACCA | | | |
| | ACACTTTTCACCAGTGGCACCGAGTCGG | | GUGGCACCGAGUCGGUGC | | | |
| | TGC | | | | | |
| 214 | TAATACGACTCACTATAGGCGAGGGCG | 131 | GCGAGGGCGAUGCCACCUAGUUUUCC | 132 | 95 | Functional |
| | ATGCCACCTAGTTTTCCAGCTAGCAATA | | AGCUAGCAAUAGCAAGUGAAAAUAA | | | Sequences |
| | GCAAGTGAAAATAAAGCTAGTCCGTTC | | AGCUAGUCCGUUCUCACCUUGACACG | | | |
| | TCACCTTGACACGGGGGCACCGAGTCG | | GGGGCACCGAGUCGGUGC | | | |
| | GTGC | | | | | |
| 215 | TAATACGACTCACTATAGGCGAGGGCG | 133 | GCGAGGGCGAUGCCACCUAGUUUCAG | 134 | 95 | Functional |
| | ATGCCACCTAGTTTCAGTGCTAGAATTA | | UGCUAGAAUUAGCAAGUUGAAAUAA | | | Sequences |
| | GCAAGTTGAAATAAGGTTATTCCGTGCC | | GGUUAUUCCGUGCCUGCCUGGACAGG | | | |
| | TGCCTGGACAGGGTGGCACCGAGTCGG | | GUGGCACCGAGUCGGUGC | | | |
| | TGC | | | | | |
| 216 | TAATACGACTCACTATAGGCGAGGGCG | 135 | GCGAGGGCGAUGCCACCUAAUUUUAC | 136 | 95 | Functional |
| | ATGCCACCTAATTTTACCGCTGGAAACA | | CGCUGGAAACAGCAAGUUAAAAUAAC | | | Sequences |
| | GCAAGTTAAAATAACGCTAGACGGTGA | | GCUAGACGGUGAUCAGCGUGCAAACG | | | |
| | TCAGCGTGCAAACGTGGCACCGAGTCG | | UGGCACCGAGUCGGUGC | | | |
| | GTGC | | | | | |
| 219 | TAATACGACTCACTATAGGCGAGGGCG | 137 | GCGAGGGCGAUGCCACCUAUUUUUAG | 138 | 95 | Functional |
| | ATGCCACCTATTTTTAGAACTAGAAATA | | AACUAGAAAUAGCAAGUUAAAAUAA | | | Sequences |
| | GCAAGTTAAAATAAGGCAAGTCCATGA | | GGCAAGUCCAUGAUCAACGGUGACCG | | | |
| | TCAACGGTGACCGTGTGGCACCGAGTC | | UGUGGCACCGAGUCGGUGC | | | |
| | GGTGC | | | | | |
| 221 | TAATACGACTCACTATAGGCGAGGGCG | 139 | GCGAGGGCGAUGCCACCUAGUUUUAG | 140 | 95 | Functional |
| | ATGCCACCTAGTTTTAGAGCTAGAAATA | | AGCUAGAAAUAGCAAGUUAAAAUAA | | | Sequences |
| | GCAAGTTAAAATAAGGTTAATTCGTTA | | GGUUAAUUCGUUAACCAACGAGAAAC | | | |
| | ACCAACGAGAAACGCGTGGCACCGAGT | | GCGUGGCACCGAGUCGGUGC | | | |
| | CGGTGC | | | | | |
| 223 | TAATACGACTCACTATAGGCGAGGGCG | 141 | GCGAGGGCGAUGCCACCUAGUUUUAU | 142 | 95 | Functional |
| | ATGCCACCTAGTTTTATAGCCAGAAATG | | AGCCAGAAAUGGCGAGUUAAAAUAG | | | Sequences |
| | GCGAGTTAAAATAGGGCCAGTCCGATA | | GGCCAGUCCGAUAUCAACUUAAUCCG | | | |
| | TCAACTTAATCCGTGGCACCGAGTCGGT | | UGGCACCGAGUCGGUGC | | | |
| | GC | | | | | |
| 224 | TAATACGACTCACTATAGGCGAGGGCG | 143 | GCGAGGGCGAUGCCACCUAGCUUUAG | 144 | 95 | Functional |
| | ATGCCACCTAGCTTTAGCGCTAGAAATA | | CGCUAGAAAUAGCAAGUUAAAGUAA | | | Sequences |
| | GCAAGTTAAAGTAAAGCGAGTCTGTGA | | AGCGAGUCUGUGAUCAACGCGAAAAC | | | |
| | TCAACGCGAAAACGTGGCACCGAGTCG | | GUGGCACCGAGUCGGUGC | | | |
| | GTGC | | | | | |
| 225 | TAATACGACTCACTATAGGCGAGGGCG | 145 | GCGAGGGCGAUGCCACCUAGUUUUAG | 146 | 95 | Functional |
| | ATGCCACCTAGTTTTAGAGCTAGAAGTA | | AGCUAGAAGUAGCAAGUUAAAAUAU | | | Sequences |
| | GCAAGTTAAAATATGGCTAGTCCGTGA | | GGCUAGUCCGUGAGCAACCCGAAGUG | | | |
| | GCAACCCGAAGTGGTGGCACCGAGTCG | | GUGGCACCGAGUCGGUGC | | | |
| | GTGC | | | | | |
| 226 | TAATACGACTCACTATAGGCGAGGGCG | 147 | GCGAGGGCGAUGCCACCUAGUUUUGG | 148 | 95 | Functional |
| | ATGCCACCTAGTTTTGGACCTAGAAATA | | ACCUAGAAAUAGGACGUCAAAAUAAG | | | Sequences |
| | GGACGTCAAAATAAGCCTAGTGCGTGC | | CCUAGUGCGUGCUCAACCUGAAAUGG | | | |
| | TCAACCTGAAATGGTGGCACCGAGTCG | | UGGCACCGAGUCGGUGC | | | |
| | GTGC | | | | | |
| 227 | TAATACGACTCACTATAGGCGAGGGCG | 149 | GCGAGGGCGAUGCCACCUAGUUUUCA | 150 | 94 | Functional |
| | ATGCCACCTAGTTTTCATGCTAGGACTA | | UGCUAGGACUAGCAAGUGAAAAUAA | | | Sequences |
| | GCAAGTGAAAATAAGTCTCGTACGTTG | | GUCUCGUACGUUGUCAACCUGAUCGG | | | |
| | TCAACCTGATCGGGTGGCACCGAGTCG | | GUGGCACCGAGUCGGUGC | | | |
| | GTGC | | | | | |
| 230 | TAATACGACTCACTATAGGCGAGGGCG | 151 | GCGAGGGCGAUGCCACCUAUUUUUAG | 152 | 95 | Functional |
| | ATGCCACCTATTTTTAGAGCCAGAAAG | | AGCCAGAAAGAGCAAGUUAAAAUAA | | | Sequences |
| | AGCAAGTTAAAATAAGGCAAGTCCGTT | | GGCAAGUCCGUUUUCAACGUAACCAC | | | |
| | TTCAACGTAACCACGTGGCACCGAGTC | | GUGGCACCGAGUCGGUGC | | | |
| | GGTGC | | | | | |
| 232 | TAATACGACTCACTATAGGCGAGGGCG | 153 | GCGAGGGCGAUGCCACCUAGUUUUAG | 154 | 96 | Functional |
| | ATGCCACCTAGTTTTAGCGCCAAAAAA | | CGCCAAAAAAAGCAAGUUAAAAUAAG | | | Sequences |
| | AGCAAGTTAAAATAAGGCGAGTCCGCT | | GCGAGUCCGCUAUCAACCUGAAACGG | | | |
| | ATCAACCTGAAACGGTGGCACCGAGTC | | UGGCACCGAGUCGGUGC | | | |
| | GGTGC | | | | | |
| 234 | TAATACGACTCACTATAGGCGAGGGCG | 155 | GCGAGGGCGAUGCCACCUAGUUUUAG | 156 | 95 | Functional |
| | ATGCCACCTAGTTTTAGAGCCAGCAATG | | AGCCAGCAAUGGCAAGUUAAAAUAGG | | | Sequences |
| | GCAAGTTAAAATAGGGCTTGTCCGTGA | | GCUUGUCCGUGAUCAACUUGAACAAG | | | |
| | TCAACTTGAACAAGGGGCACCGAGTCG | | GGGCACCGAGUCGGUGC | | | |
| | GTGC | | | | | |
| 235 | TAATACGACTCACTATAGGCGAGGGCG | 157 | GCGAGGGCGAUGCCACCUAGUUUUCG | 158 | 95 | Functional |
| | ATGCCACCTAGTTTTCGATCAAGAAATT | | AUCAAGAAAUUGCAAGUGAAAACAA | | | Sequences |
| | GCAAGTGAAAACAAGGCAATCCCGTAC | | GGCAAUCCCGUACCCAACCUGAAACG | | | |
| | CCAACCTGAAACGGTGGCACCGAGTCG | | GUGGCACCGAGUCGGUGC | | | |
| | GTGC | | | | | |
| 239 | TAATACGACTCACTATAGGCGAGGGCG | 159 | GCGAGGGCGAUGCCACCUAGUUUUAG | 160 | 95 | Functional |
| | ATGCCACCTAGTTTTAGAGCTAGAAATA | | AGCUAGAAAUAGCAAGUUAAAAUAC | | | Sequences |
| | GCAAGTTAAAATACGACTAGTTCATTA | | GACUAGUUCAUUAAUAGCAUGAAAAC | | | |
| | ATAGCATGAAAACGTGGCACCGAGTCG | | GUGGCACCGAGUCGGUGC | | | |
| | GTGC | | | | | |
| 240 | TAATACGACTCACTATAGGCGAGGGCG | 161 | GCGAGGGCGAUGCCACCUAGUUUUCG | 162 | 95 | Functional |
| | ATGCCACCTAGTTTTCGAGCCAGAAATG | | AGCCAGAAAUGGCAAGUGAAAAUAA | | | Sequences |
| | GCAAGTGAAAATAAGGCAAGTCCGTTA | | GGCAAGUCCGUUAGCGACUGUUCACA | | | |
| | GCGACTGTTCACAGTGGCACCGAGTCG | | GUGGCACCGAGUCGGUGC | | | |
| | GTGC | | | | | |
| 243 | TAATACGACTCACTATAGGCGAGGGCG | 163 | GCGAGGGCGAUGCCACCUAGUUUGAG | 164 | 95 | Functional |
| | ATGCCACCTAGTTTGAGAAAGTGAACC | | AAAGUGAACCUUCAAGUUCAAAUAAG | | | Sequences |
| | TTCAAGTTCAAATAAGGTTTGTCCGGTA | | GUUUGUCCGGUAUCAACUGGAAACAG | | | |
| | TCAACTGGAAACAGTGGCACCGAGTCG | | UGGCACCGAGUCGGUGC | | | |
| | GTGC | | | | | |
| 249 | TAATACGACTCACTATAGGCGAGGGCG | 165 | GCGAGGGCGAUGCCACCUAAUUUUUG | 166 | 95 | Functional |
| | ATGCCACCTAATTTTTGCGCTAGTAATA | | CGCUAGUAAUAGCAAGUAAAAAUAA | | | Sequences |
| | GCAAGTAAAAATAAGACTGGTCCGTTA | | GACUGGUCCGUUACCAACCUGGAAGG | | | |
| | CCAACCTGGAAGGGTGGCACCGAGTCG | | GUGGCACCGAGUCGGUGC | | | |
| | GTGC | | | | | |
| 251 | TAATACGACTCACTATAGGCGAGGGCG | 167 | GCGAGGGCGAUGCCACCUAGUUUUGG | 168 | 95 | Functional |
| | ATGCCACCTAGTTTTGGAGCTAGTTTGA | | AGCUAGUUUGAGCAAGUCAAAAUAA | | | Sequences |
| | GCAAGTCAAAATAAGGCGAGTCCGTTA | | GGCGAGUCCGUUAUUAACUUGAACAU | | | |
| | TTAACTTGAACATGTGGCACCGAGTCG | | GUGGCACCGAGUCGGUGC | | | |
| | GTGC | | | | | |
| 254 | TAATACGACTCACTATAGGCGAGGGCG | 169 | GCGAGGGCGAUGCCACCUAGUUUUAG | 170 | 95 | Functional |
| | ATGCCACCTAGTTTTAGAGCTAGCAATA | | AGCUAGCAAUAGCAAGUUAGAAUAA | | | Sequences |
| | GCAAGTTAGAATAAGGCGAGACCGTTA | | GGCGAGACCGUUAUCAGCUGGAACCA | | | |
| | TCAGCTGGAACCAGTGGCACCGAGTCG | | GUGGCACCGAGUCGGUGC | | | |
| | GTGC | | | | | |
| 259 | TAATACGACTCACTATAGGCGAGGGCG | 171 | GCGAGGGCGAUGCCACCUAGCUUUAG | 172 | 95 | Functional |
| | ATGCCACCTAGCTTTAGAGCTAAAAATT | | AGCUAAAAAUUAGCAAGUUAAAGUC | | | Sequences |
| | AGCAAGTTAAAGTCAGGCTAGTCCGTG | | AGGCUAGUCCGUGCGGAACGUGCCCC | | | |
| | CGGAACGTGCCCCTGTGGCACCGAGTC | | UGUGGCACCGAGUCGGUGC | | | |
| | GGTGC | | | | | |
| N/A | TAATACGACTCACTATAGGCGAGGGCG | 173 | GCGAGGGCGAUGCCACCUAGUUUUAC | 174 | 95 | MiRNA |
| | ATGCCACCTAGTTTTACCGCTAGAAATA | | CGCUAGAAAUAGCAAGUUAAAAUAA | | | Functional |
| | GCAAGTTAAAATAAGGCTAGACCGGAA | | GGCUAGACCGGAAUAACCAUGCAAAU | | | Sequences |
| | TAACCATGCAAATGTGGCACCGAGTCG | | GUGGCACCGAGUCGGUGC | | | |
| | GTGC | | | | | |
| N/A | TAATACGACTCACTATAGGCGAGGGCG | 175 | GCGAGGGCGAUGCCACCUAGUUUAAU | 176 | 95 | MiRNA |
| | ATGCCACCTAGTTTAATAGCGAGTAATC | | AGCGAGUAAUCGCAUGUUUAAAUAA | | | Functional |
| | GCATGTTTAAATAAGGCTAGACCGGTA | | GGCUAGACCGGUAACAGAUUGAAUCA | | | Sequences |
| | ACAGATTGAATCAGTGGCACCGAGTCG | | GUGGCACCGAGUCGGUGC | | | |
| | GTGC | | | | | |
| N/A | TAATACGACTCACTATAGGCGAGGGCG | 177 | GCGAGGGCGAUGCCACCUAGUUUUAG | 178 | 95 | MiRNA |
| | ATGCCACCTAGTTTTAGCGCTAGTAATA | | CGCUAGUAAUAGCAAGUUGAAAUAA | | | Functional |
| | GCAAGTTGAAATAAGGATAATCCGTTA | | GGAUAAUCCGUUACCAUCUGUGCACA | | | Sequences |
| | CCATCTGTGCACAGTGGCACCGAGTCG | | GUGGCACCGAGUCGGUGC | | | |
| | GTGC | | | | | |
| N/A | TAATACGACTCACTATAGGCGAGGGCG | 179 | GCGAGGGCGAUGCCACCUAGUUUUAG | 180 | 94 | MiRNA |
| | ATGCCACCTAGTTTTAGTGCTAGAAATG | | UGCUAGAAAUGGCCAGUUAAAAUAA | | | Functional |
| | GCCAGTTAAAATAAGGCCAGCGCGCTA | | GGCCAGCGCGCUACCAGCGUACAUAC | | | Sequences |
| | CCAGCGTACATACGTGGCACCGAGTCG | | GUGGCACCGAGUCGGUGC | | | |
| | GTGC | | | | | |
| N/A | TAATACGACTCACTATAGGCGAGGGCG | 181 | GCGAGGGCGAUGCCACCUAGUUUUAG | 182 | 95 | MiRNA |
| | ATGCCACCTAGTTTTAGTGCTCGAAAGA | | UGCUCGAAAGAGAAAGUUAAAAUAA | | | Functional |
| | GAAAGTTAAAATAAGAACATTTCGCGA | | GAACAUUUCGCGAUCACCGUUGAUAC | | | Sequences |
| | TCACCGTTGATACGTGGCACCGAGTCG | | GUGGCACCGAGUCGGUGC | | | |
| | GTGC | | | | | |
| N/A | TAATACGACTCACTATAGGCGAGGGCG | 183 | GCGAGGGCGAUGCCACCUAGUUUUAG | 184 | 96 | MiRNA |
| | ATGCCACCTAGTTTTAGGTCTAGAAATA | | GUCUAGAAAUAGCGAGUUAAAAUAA | | | Functional |
| | GCGAGTTAAAATAAGGACAATCCGTAC | | GGACAAUCCGUACGCAACGGCAAAAC | | | Sequences |
| | GCAACGGCAAAACGTGGCACCGAGTCG | | GUGGCACCGAGUCGGUGC | | | |
| | GTGC | | | | | |
| N/A | TAATACGACTCACTATAGGCGAGGGCG | 185 | GCGAGGGCGAUGCCACCUAGUUUUGC | 186 | 95 | MiRNA |
| | ATGCCACCTAGTTTTGCAGCTAGAATTA | | AGCUAGAAUUAGCAUGUCAAAAUAA | | | Functional |
| | GCATGTCAAAATAAGGTTCCTCCGGTG | | GGUUCCUCCGGUGACAACGUGAAUAC | | | Sequences |
| | ACAACGTGAATACGTGGCACCGAGTCG | | GUGGCACCGAGUCGGUGC | | | |
| | GTGC | | | | | |
| N/A | TAATACGACTCACTATAGGCGAGGGCG | 187 | GCGAGGGCGAUGCCACCUAUUUUCAG | 188 | 95 | MiRNA |
| | ATGCCACCTATTTTCAGTGCTAGAATTA | | UGCUAGAAUUAGCAAGUUGAAAUAA | | | Functional |
| | GCAAGTTGAAATAAGGTTATTCCGTGCC | | GGUUAUUCCGUGCCUGCCUGGACAGG | | | Sequences |
| | TGCCTGGACAGGGTGGCACCGAGTCGG | | GUGGCACCGAGUCGGUGC | | | |
| | TGC | | | | | |
| N/A | TAATACGACTCACTATAGGCGAGGGCG | 189 | GCGAGGGCGAUGCCACCUAGUUUGAG | 190 | 95 | MiRNA |
| | ATGCCACCTAGTTTGAGAGCTAAAAAT | | AGCUAAAAAUAGCAAGUUCAAAUAA | | | Functional |
| | AGCAAGTTCAAATAAGGTTAGACCGTA | | GGUUAGACCGUAAUUUCGUUGUACAU | | | Sequences |
| | ATTTCGTTGTACATGTGGCACCGAGTCG | | GUGGCACCGAGUCGGUGC | | | |
| | GTGC | | | | | |
| N/A | TAATACGACTCACTATAGGCGAGGGCG | 191 | GCGAGGGCGAUGCCACCUAGUCUUAG | 192 | 95 | MiRNA |
| | ATGCCACCTAGTCTTAGAGCTAGACCTA | | AGCUAGACCUAGCACGUUAAAAUAAG | | | Functional |
| | GCACGTTAAAATAAGGCGAGTTCGTTA | | GCGAGUUCGUUAUCAACCAUUUCGAG | | | Sequences |
| | TCAACCATTTCGAGTGGCACCGAGTCG | | UGGCACCGAGUCGGUGC | | | |
| | GTGC | | | | | |
| N/A | TAATACGACTCACTATAGGCGAGGGCG | 193 | GCGAGGGCGAUGCCACCUAGUUUUAG | 194 | 95 | MiRNA |
| | ATGCCACCTAGTTTTAGTGCTAAAAATA | | UGCUAAAAAUACCGUGUUAAAAUAA | | | Functional |
| | CCGTGTTAAAATAAGGCATGTTCGTTAG | | GGCAUGUUCGUUAGCAACAUUAUUGU | | | Sequences |
| | CAACATTATTGTGCGGCACCGAGTCGGT | | GCGGCACCGAGUCGGUGC | | | |
| | GC | | | | | |
| N/A | TAATACGACTCACTATAGGCGAGGGCG | 195 | GCGAGGGCGAUGCCACCUAGUUUUCG | 196 | 96 | MiRNA |
| | ATGCCACCTAGTTTTCGAGCCAGAAATG | | AGCCAGAAAUGGCAAGUGAAAAUAA | | | Functional |
| | GCAAGTGAAAATAAGGCAAGTCTGTTA | | GGCAAGUCUGUUAGCGACUGUUCACA | | | Sequences |
| | GCGACTGTTCACAGTGGCACCGAGTCG | | GUGGCACCGAGUCGGUGC | | | |
| | GTGC | | | | | |
| N/A | TAATACGACTCACTATAGGCGAGGGCG | 197 | GCGAGGGCGAUGCCACCUAGUUUUAG | 198 | 95 | MiRNA |
| | ATGCCACCTAGTTTTAGAGCTAAAGATA | | AGCUAAAGAUAGCAAGUUAAAAUAA | | | Functional |
| | GCAAGTTAAAATAAGACGAATTCGTTA | | GACGAAUUCGUUACCUACUUCCACUG | | | Sequences |
| | CCTACTTCCACTGGTGGCACCGAGTCGG | | GUGGCACCGAGUCGGUGC | | | |
| | TGC | | | | | |
| N/A | TAATACGACTCACTATAGGCGAGGGCG | 199 | GCGAGGGCGAUGCCACCUAUAUUUAG | 200 | 95 | MiRNA |
| | ATGCCACCTATATTTAGAGGTCGAAAA | | AGGUCGAAAAACCAAGUUAAAAUAA | | | Functional |
| | ACCAAGTTAAAATAAGGTTAAACCGTT | | GGUUAAACCGUUAUAACCUGGAACAG | | | Sequences |
| | ATAACCTGGAACAGTTGGCACCGAGTC | | UUGGCACCGAGUCGGUGC | | | |
| | GGTGC | | | | | |
| 237 | TAATACGACTCACTATAGGCGAGGGCG | 201 | GCGAGGGCGAUGCCACCUAGAGUUGA | 202 | 95 | Cleavage |
| | ATGCCACCTAGAGTTGAAACAACGAAT | | AACAACGAAUAGCAUAUUUCAAUAUG | | | Incapable |
| | AGCATATTTCAATATGTTTATTCCGGTG | | UUUAUUCCGGUGCAACGUUGUACACG | | | Sequences |
| | CAACGTTGTACACGTGGCACCGAGTCG | | UGGCACCGAGUCGGUGC | | | |
| | GTGC | | | | | |
| 250 | TAATACGACTCACTATAGGCGAGGGCG | 203 | GCGAGGGCGAUGCCACCUAGCGUGAG | 204 | 95 | Cleavage |
| | ATGCCACCTAGCGTGAGCACTCGAACTT | | CACUCGAACUUGCAAGUAUCAACAAG | | | Incapable |
| | GCAAGTATCAACAAGGTGAGTCCCCTG | | GUGAGUCCCCUGCCAUCGUGAAACGG | | | Sequences |
| | CCATCGTGAAACGGTGGCACCGAGTCG | | UGGCACCGAGUCGGUGC | | | |
| | GTGC | | | | | |
| 257 | TAATACGACTCACTATAGGCGAGGGCG | 205 | GCGAGGGCGAUGCCACCUACCUUAGA | 206 | 95 | Cleavage |
| | ATGCCACCTACCTTAGATGCTCGTAGTT | | UGCUCGUAGUUGAAACUUCGAGUAGG | | | Incapable |
| | GAAACTTCGAGTAGGACGGTGCCCTTG | | ACGGUGCCCUUGUCACCUUGAGUGGU | | | Sequences |
| | TCACCTTGAGTGGTGGGCACCGAGTCG | | GGGCACCGAGUCGGUGC | | | |
| | GTGC | | | | | |
| 211 | TAATACGACTCACTATAGGCGAGGGCG | 207 | GCGAGGGCGAUGCCACCUAGUUUUCA | 208 | 95 | Cleavage |
| | ATGCCACCTAGTTTTCAATCTAGAAGCG | | AUCUAGAAGCGACAUCAUACCCUAAG | | | Incapable |
| | ACATCATACCCTAAGTCTGGTCCTTCAT | | UCUGGUCCUUCAUAAACUUGCCCCUG | | | Sequences |
| | AAACTTGCCCCTGCGGCACCGAGTCGG | | CGGCACCGAGUCGGUGC | | | |
| | TGC | | | | | |
| 228 | TAATACGACTCACTATAGGCGAGGGCG | 209 | GCGAGGGCGAUGCCACCUAUUUUUCG | 210 | 95 | Cleavage |
| | ATGCCACCTATTTTTCGATGTAGAACTA | | AUGUAGAACUACAAAGUGAAAAGAG | | | Incapable |
| | CAAAGTGAAAAGAGGTCTAGTCACCTA | | GUCUAGUCACCUAUCCCCCUGUGCCG | | | Sequences |
| | TCCCCCTGTGCCGGCGGCACCGAGTCG | | GCGGCACCGAGUCGGUGC | | | |
| | GTGC | | | | | |
| 253 | TAATACGACTCACTATAGGCGAGGGCG | 211 | GCGAGGGCGAUGCCACCUAAACGUGG | 212 | 95 | Cleavage |
| | ATGCCACCTAAACGTGGTGCTAGATAT | | UGCUAGAUAUAAACUGUAAAUAGAA | | | Incapable |
| | AAACTGTAAATAGAAAGTTGGTCTTTG | | AGUUGGUCUUUGGUGACGCUGUUCUC | | | Sequences |
| | GTGACGCTGTTCTCGCGGCACCGAGTCG | | GCGGCACCGAGUCGGUGC | | | |
| | GTGC | | | | | |
| 241 | TAATACGACTCACTATAGGCGAGGGCG | 213 | GCGAGGGCGAUGCCACCUAGUCAACU | 214 | 95 | Cleavage |
| | ATGCCACCTAGTCAACTAGCTAGAACT | | AGCUAGAACUAACAAGUGAAGAGAA | | | Incapable |
| | AACAAGTGAAGAGAATTCGAGTTAGTT | | UUCGAGUUAGUUUUCACCGCUAUCCC | | | Sequences |
| | TTCACCGCTATCCCGTGGCACCGAGTCG | | GUGGCACCGAGUCGGUGC | | | |
| | GTGC | | | | | |
| 220 | TAATACGACTCACTATAGGCGAGGGCG | 215 | GCGAGGGCGAUGCCACCUAUUGUAGU | 216 | 93 | Cleavage |
| | ATGCCACCTATTGTAGTTGCCAGAAACA | | UGCCAGAAACAACAAGUCAAGAUAGC | | | Incapable |
| | ACAAGTCAAGATAGCGAGTCCGTCCTC | | GAGUCCGUCCUCACCCGGUGACCCCG | | | Sequences |
| | ACCCGGTGACCCCGGCACCGAGTCGGT | | GCACCGAGUCGGUGC | | | |
| | GC | | | | | |
| 246 | TAATACGACTCACTATAGGCGAGGGCG | 217 | GCGAGGGCGAUGCCACCUAGUUCUAG | 218 | 95 | Cleavage |
| | ATGCCACCTAGTTCTAGATCTATCAACA | | AUCUAUCAACAGCAAGUUGAAAGAAA | | | Incapable |
| | GCAAGTTGAAAGAAAGTTAGAACGATG | | GUUAGAACGAUGGGAACUUUCACCCU | | | Sequences |
| | GGAACTTTCACCCTCGGCACCGAGTCG | | CGGCACCGAGUCGGUGC | | | |
| | GTGC | | | | | |
| 218 | TAATACGACTCACTATAGGCGAGGGCG | 219 | GCGAGGGCGAUGCCACCUAGUGGUCG | 220 | 95 | Cleavage |
| | ATGCCACCTAGTGGTCGGACTATACATA | | GACUAUACAUAGCUAGUUCCGAUAAG | | | Incapable |
| | GCTAGTTCCGATAAGTCTAGATCGCAG | | UCUAGAUCGCAGGCAAACUGCUCCGG | | | Sequences |
| | GCAAACTGCTCCGGTGGCACCGAGTCG | | UGGCACCGAGUCGGUGC | | | |
| | GTGC | | | | | |
| 255 | TAATACGACTCACTATAGGCGAGGGCG | 221 | GCGAGGGCGAUGCCACCUAGUGGUCG | 222 | 94 | Cleavage |
| | ATGCCACCTAGTGGTCGGACTATACATA | | GACUAUACAUAGCUAGUUCCGAUAAG | | | Incapable |
| | GCTAGTTCCGATAAGTCTAGATCGCAG | | UCUAGAUCGCAGGCAACUGCUCCGGU | | | Sequences |
| | GCAACTGCTCCGGTGGCACCGAGTCGG | | GGCACCGAGUCGGUGC | | | |
| | TGC | | | | | |
| 245 | TAATACGACTCACTATAGGCGAGGGCG | 223 | GCGAGGGCGAUGCCACCUAGGGCCUG | 224 | 52 | Cleavage |
| | ATGCCACCTAGGGCCTGAGCACTGCAC | | AGCACUGCACGGCACCGAGUCGGUGC | | | Incapable |
| | GGCACCGAGTCGGTGC | | | | | Sequences |
| 244 | TAATACGACTCACTATAGGCGAGGGCG | 225 | GCGAGGGCGAUGCCACCUAGUGUUUG | 226 | 95 | Cleavage |
| | ATGCCACCTAGTGTTTGAAATCGCAATA | | AAAUCGCAAUAGCACGAUCACCUAAU | | | Incapable |
| | GCACGATCACCTAATGCTAGTCCGCGA | | GCUAGUCCGCGACAAACGUGGUGCUG | | | Sequences |
| | CAAACGTGGTGCTGCCGCACCGAGTCG | | CCGCACCGAGUCGGUGC | | | |
| | GTGC | | | | | |
| 236 | TAATACGACTCACTATAGGCGAGGGCG | 227 | GCGAGGGCGAUGCCACCUAGUUACUA | 228 | 87 | Cleavage |
| | ATGCCACCTAGTTACTAATCGCTCGTGT | | AUCGCUCGUGUAACUUAGCGUAACCU | | | Incapable |
| | AACTTAGCGTAACCTTCCGCTCACCTGG | | UCCGCUCACCUGGUGCCGUGGCACCG | | | Sequences |
| | TGCCGTGGCACCGAGTCGGTGC | | AGUCGGUGC | | | |
| 248 | TAATACGACTCACTATAGGCGAGGGCG | 229 | GCGAGGGCGAUGCCACCUAGGUUUAG | 230 | 96 | Cleavage |
| | ATGCCACCTAGGTTTAGACTTAGATACG | | ACUUAGAUACGUUAAGUUAUAAACCC | | | Incapable |
| | TTAAGTTATAAACCCCCCAGTGCGGTGT | | CCCAGUGCGGUGUGAAGUGGAACGCC | | | Sequences |
| | GAAGTGGAACGCCTGGGCACCGAGTCG | | UGGGCACCGAGUCGGUGC | | | |
| | GTGC | | | | | |
| 204 | TAATACGACTCACTATAGGCGAGGGCG | 231 | GCGAGGGCGAUGCCACCUAGGUAUAG | 232 | 95 | Cleavage |
| | ATGCCACCTAGGTATAGAGCTAGAAAT | | AGCUAGAAAUAGCCGCCCAGAAUCAG | | | Incapable |
| | AGCCGCCCAGAATCAGCCCAGTGCGTT | | CCCAGUGCGUUAUUAACCGUUUGCGU | | | Sequences |
| | ATTAACCGTTTGCGTGGGCACCGAGTCG | | GGGCACCGAGUCGGUGC | | | |
| | GTGC | | | | | |
| 233 | TAATACGACTCACTATAGGCGAGGGCG | 233 | GCGAGGGCGAUGCCACCUAGCGCUAA | 234 | 95 | Cleavage |
| | ATGCCACCTAGCGCTAAAGTTAGACTTA | | AGUUAGACUUAGCAAGUUAGGUUCCG | | | Incapable |
| | GCAAGTTAGGTTCCGTCTGTTCCCGAGT | | UCUGUUCCCGAGUCAACUUGGUGCAG | | | Sequences |
| | CAACTTGGTGCAGTGGCACCGAGTCGG | | UGGCACCGAGUCGGUGC | | | |
| | TGC | | | | | |
| 258 | TAATACGACTCACTATAGGCGAGGGCG | 235 | GCGAGGGCGAUGCCACCUACUUGUAG | 236 | 94 | Cleavage |
| | ATGCCACCTACTTGTAGAGATACGAAT | | AGAUACGAAUAGCAGUGUAAGUUUG | | | Incapable |
| | AGCAGTGTAAGTTTGCGCTAGTCAACTC | | CGCUAGUCAACUCGCAAUUGGUGCCG | | | Sequences |
| | GCAATTGGTGCCGTGGCACCGAGTCGG | | UGGCACCGAGUCGGUGC | | | |
| | TGC | | | | | |
| 231 | TAATACGACTCACTATAGGCGAGGGCG | 237 | GCGAGGGCGAUGCCACCUAUGUAUGC | 238 | 95 | Cleavage |
| | ATGCCACCTATGTATGCAACTAACTATA | | AACUAACUAUAGCAUACUAGAGAUUG | | | Incapable |
| | GCATACTAGAGATTGGCAAGTCGCTAC | | GCAAGUCGCUACUUACCUAGGUGCCG | | | Sequences |
| | TTACCTAGGTGCCGTGGCACCGAGTCG | | UGGCACCGAGUCGGUGC | | | |
| | GTGC | | | | | |
| 222 | TAATACGACTCACTATAGGCGAGGGCG | 239 | GCGAGGGCGAUGCCACCUACGUAUGC | 240 | 95 | Cleavage |
| | ATGCCACCTACGTATGCAACTAACTATA | | AACUAACUAUAGCAUACUAGAGAUUG | | | Incapable |
| | GCATACTAGAGATTGGCAAGTCGCTAC | | GCAAGUCGCUACUUACCUAGGUGCCG | | | Sequences |
| | TTACCTAGGTGCCGTGGCACCGAGTCG | | UGGCACCGAGUCGGUGC | | | |
| | GTGC | | | | | |
| 229 | TAATACGACTCACTATAGGCGAGGGCG | 241 | GCGAGGGCGAUGCCACCUAGCUCGAU | 242 | 95 | Cleavage |
| | ATGCCACCTAGCTCGATAGCTATTTAGA | | AGCUAUUUAGAUAAAGUUAAAUUAA | | | Incapable |
| | TAAAGTTAAATTAAGGCTACGCGGGTA | | GGCUACGCGGGUAACAACUCGACCCC | | | Sequences |
| | ACAACTCGACCCCGTGGCACCGAGTCG | | GUGGCACCGAGUCGGUGC | | | |
| | GTGC | | | | | |
| 242 | TAATACGACTCACTATAGGCGAGGGCG | 243 | GCGAGGGCGAUGCCACCUAGUUUUAG | 244 | 95 | Cleavage |
| | ATGCCACCTAGTTTTAGAGCTAAAAATA | | AGCUAAAAAUAGCAAGUUAAAAUAU | | | Incapable |
| | GCAAGTTAAAATATGGGTGCTCCCATGT | | GGGUGCUCCCAUGUCGUCUCGACUGC | | | Sequences |
| | CGTCTCGACTGCGGGGCACCGAGTCGG | | GGGGCACCGAGUCGGUGC | | | |
| | TGC | | | | | |
| 256 | TAATACGACTCACTATAGGCGAGGGCG | 245 | GCGAGGGCGAUGCCACCUAUUGUUCG | 246 | 95 | Cleavage |
| | ATGCCACCTATTGTTCGATCATGAAACA | | AUCAUGAAACAGCAAGGUAAAACAUC | | | Incapable |
| | GCAAGGTAAAACATCGCAAGTTCGATA | | GCAAGUUCGAUAACGGUUACGGUGCG | | | Sequences |
| | ACGGTTACGGTGCGTGGCACCGAGTCG | | UGGCACCGAGUCGGUGC | | | |
| | GTGC | | | | | |
| 247 | TAATACGACTCACTATAGGCGAGGGCG | 247 | GCGAGGGCGAUGCCACCUAGUUUUAG | 248 | 95 | Cleavage |
| | ATGCCACCTAGTTTTAGAGCTTTCAGTA | | AGCUUUCAGUAGCAAGUUAAAACAUG | | | Incapable |
| | GCAAGTTAAAACATGGCTAGTCCGTTG | | GCUAGUCCGUUGCCAUCUGGUGCGCG | | | Sequences |
| | CCATCTGGTGCGCGTGGCACCGAGTCG | | UGGCACCGAGUCGGUGC | | | |
| | GTGC | | | | | |
| 252 | TAATACGACTCACTATAGGCGAGGGCG | 249 | GCGAGGGCGAUGCCACCUAGUUUUAG | 250 | 94 | Cleavage |
| | ATGCCACCTAGTTTTAGAGCGGAAATC | | AGCGGAAAUCGCAAGUUAAAAUAAG | | | Incapable |
| | GCAAGTTAAAATAAGGCTGGTCAATCC | | GCUGGUCAAUCCUCAAGGUGCUCGCG | | | Sequences |
| | TCAAGGTGCTCGCGTGGCACCGAGTCG | | UGGCACCGAGUCGGUGC | | | |
| | GTGC | | | | | |
| 217 | TAATACGACTCACTATAGGCGAGGGCG | 251 | GCGAGGGCGAUGCCACCUAGCUUAAG | 252 | 95 | Cleavage |
| | ATGCCACCTAGCTTAAGTGCAAGAAAG | | UGCAAGAAAGUGCAAAUUAGGACAA | | | Incapable |
| | TGCAAATTAGGACAAGGCTATCAAGTC | | GGCUAUCAAGUCCUCAACCUCAACUG | | | Sequences |
| | CTCAACCTCAACTGGTGGCACCGAGTC | | GUGGCACCGAGUCGGUGC | | | |
| | GGTGC | | | | | |
| 238 | TAATACGACTCACTATAGGCGAGGGCG | 253 | GCGAGGGCGAUGCCACCUAGUCUUAG | 254 | 95 | Cleavage |
| | ATGCCACCTAGTCTTAGAGCTAGAAAT | | AGCUAGAAAUAGCAAGUUAAGAUAA | | | Incapable |
| | AGCAAGTTAAGATAAGGCTAGTCCATC | | GGCUAGUCCAUCGUCCACCGCAGCCG | | | Sequences |
| | GTCCACCGCAGCCGGTGGCACCGAGTC | | GUGGCACCGAGUCGGUGC | | | |
| | GGTGC | | | | | |
| 3 | TAATACGACTCACTATAGGCGAGGGCG | 255 | GCGAGGGCGAUGCCACCUAGGUUAAC | 256 | 95 | Cleavage |
| | ATGCCACCTAGGTTAACCGCTCTAAACA | | CGCUCUAAACAGCGAGUGAAUUCGAG | | | Incapable |
| | GCGAGTGAATTCGAGGCTTGTGCACAA | | GCUUGUGCACAAUCACCCGUUAUCGG | | | Sequences |
| | TCACCCGTTATCGGTGGCACCGAGTCGG | | UGGCACCGAGUCGGUGC | | | |
| | TGC | | | | | |
| 5 | TAATACGACTCACTATAGGCGAGGGCG | 257 | GCGAGGGCGAUGCCACCUAGCUCGAU | 258 | 95 | Cleavage |
| | ATGCCACCTAGCTCGATAGCTATTTAGA | | AGCUAUUUAGAUAAAGUUAAAUUAA | | | Incapable |
| | TAAAGTTAAATTAAGGCTACGCGGGTA | | GGCUACGCGGGUAACAACUCGACCCC | | | Sequences |
| | ACAACTCGACCCCGTGGCACCGAGTCG | | GUGGCACCGAGUCGGUGC | | | |
| | GTGC | | | | | |
| 9 | TAATACGACTCACTATAGGCGAGGGCG | 259 | GCGAGGGCGAUGCCACCUAGCUUAAG | 260 | 95 | Cleavage |
| | ATGCCACCTAGCTTAAGTGCAAGAAAG | | UGCAAGAAAGUGCAAAUUAGGACAA | | | Incapable |
| | TGCAAATTAGGACAAGGCTATCAAGTC | | GGCUAUCAAGUCCUCAACCUCAACUG | | | Sequences |
| | CTCAACCTCAACTGGTGGCACCGAGTC | | GUGGCACCGAGUCGGUGC | | | |
| | GGTGC | | | | | |
| 12 | TAATACGACTCACTATAGGCGAGGGCG | 261 | GCGAGGGCGAUGCCACCUAGUCUUGG | 262 | 95 | Cleavage |
| | ATGCCACCTAGTCTTGGCAGCCGACAG | | CAGCCGACAGCAGUAAGUUAACUUAU | | | Incapable |
| | CAGTAAGTTAACTTATGTTTAGTCTGAC | | GUUUAGUCUGACCUUACCCUUUACCG | | | Sequences |
| | CTTACCCTTTACCGGTGGCACCGAGTCG | | GUGGCACCGAGUCGGUGC | | | |
| | GTGC | | | | | |
| 13 | TAATACGACTCACTATAGGCGAGGGCG | 263 | GCGAGGGCGAUGCCACCUAGCUUUCG | 264 | 95 | Cleavage |
| | ATGCCACCTAGCTTTCGAGCCAGTGACG | | AGCCAGUGACGGUAAGUGAAAUCGAG | | | Incapable |
| | GTAAGTGAAATCGAGGCTAGTCCGTTA | | GCUAGUCCGUUAGCCCAUUGAACUGG | | | Sequences |
| | GCCCATTGAACTGGTGGCACCGAGTCG | | UGGCACCGAGUCGGUGC | | | |
| | GTGC | | | | | |
| 14 | TAATACGACTCACTATAGGCGAGGGCG | 265 | GCGAGGGCGAUGCCACCUAGUGGUCG | 266 | 95 | Cleavage |
| | ATGCCACCTAGTGGTCGGACTATACATA | | GACUAUACAUAGCUAGUUCCGAUAAG | | | Incapable |
| | GCTAGTTCCGATAAGTCTAGATCGCAG | | UCUAGAUCGCAGGCAAACUGCUCCGG | | | Sequences |
| | GCAAACTGCTCCGGTGGCACCGAGTCG | | UGGCACCGAGUCGGUGC | | | |
| | GTGC | | | | | |
| 17 | TAATACGACTCACTATAGGCGAGGGCG | 267 | GCGAGGGCGAUGCCACCUAGUCAACU | 268 | 95 | Cleavage |
| | ATGCCACCTAGTCAACTAGCTAGAACT | | AGCUAGAACUAACAAGUGAAGAGAA | | | Incapable |
| | AACAAGTGAAGAGAATTCGAGTTAGTT | | UUCGAGUUAGUUUUCACCGCUAUCCC | | | Sequences |
| | TTCACCGCTATCCCGTGGCACCGAGTCG | | GUGGCACCGAGUCGGUGC | | | |
| | GTGC | | | | | |
| 20 | TAATACGACTCACTATAGGCGAGGGCG | 269 | GCGAGGGCGAUGCCACCUAGCGAUCG | 270 | 95 | Cleavage |
| | ATGCCACCTAGCGATCGAGCTAGACAT | | AGCUAGACAUCGCAAGUUCGCAAAAU | | | Incapable |
| | CGCAAGTTCGCAAAATACGAGTGCACC | | ACGAGUGCACCAGGCACUUCAGAGGG | | | Sequences |
| | AGGCACTTCAGAGGGTGGCACCGAGTC | | UGGCACCGAGUCGGUGC | | | |
| | GGTGC | | | | | |
| 24 | TAATACGACTCACTATAGGCGAGGGCG | 27 | GCGAGGGCGAUGCCACCUAGUGUUCG | 272 | 95 | Cleavage |
| | ATGCCACCTAGTGTTCGAGCTAGGCTTA | | AGCUAGGCUUAGCAAGUGAACAUUAG | | | Incapable |
| | GCAAGTGAACATTAGGCGAGTCCGTTA | | GCGAGUCCGUUAUCAACUUGGAACAG | | | Sequences |
| | TCAACTTGGAACAGTGGCACCGAGTCG | | UGGCACCGAGUCGGUGC | | | |
| | GTGC | | | | | |
| 26 | TAATACGACTCACTATAGGCGAGGGCG | 273 | GCGAGGGCGAUGCCACCUAGCCAUAG | 274 | 95 | Cleavage |
| | ATGCCACCTAGCCATAGAGCTAGACAT | | AGCUAGACAUACCAAGGCAAAACAUU | | | Incapable |
| | ACCAAGGCAAAACATTGCCAGTGCGCT | | GCCAGUGCGCUACCAACCAAAAGCGG | | | Sequences |
| | ACCAACCAAAAGCGGTGGCACCGAGTC | | UGGCACCGAGUCGGUGC | | | |
| | GGTGC | | | | | |
| 27 | TAATACGACTCACTATAGGCGAGGGCG | 275 | GCGAGGGCGAUGCCACCUAAUUCUGG | 276 | 95 | Cleavage |
| | ATGCCACCTAATTCTGGTGACATTAAAC | | UGACAUUAAACGCCAGUUCGCGUUAU | | | Incapable |
| | GCCAGTTCGCGTTATGCAATCCCGTTCC | | GCAAUCCCGUUCCAUACUUGAACCGG | | | Sequences |
| | ATACTTGAACCGGTGGCACCGAGTCGG | | UGGCACCGAGUCGGUGC | | | |
| | TGC | | | | | |
| 29 | TAATACGACTCACTATAGGCGAGGGCG | 277 | GCGAGGGCGAUGCCACCUAGUUAUAG | 278 | 95 | Cleavage |
| | ATGCCACCTAGTTATAGTGTTAGGAATG | | UGUUAGGAAUGGCCCCCUAGCAUUAC | | | Incapable |
| | GCCCCCTAGCATTACGTGTCTCCGCCAT | | GUGUCUCCGCCAUUAAUUACUACACG | | | Sequences |
| | TAATTACTACACGGGGCACCGAGTCGG | | GGGCACCGAGUCGGUGC | | | |
| | TGC | | | | | |
| 30 | TAATACGACTCACTATAGGCGAGGGCG | 279 | GCGAGGGCGAUGCCACCUAUGUGAGG | 280 | 95 | Cleavage |
| | ATGCCACCTATGTGAGGATTCAAAAAC | | AUUCAAAAACAUCCAUGUACAUUAAG | | | Incapable |
| | ATCCATGTACATTAAGCCTAGTCAGTTA | | CCUAGUCAGUUACCAAACCCCUGCGG | | | Sequences |
| | CCAAACCCCTGCGGTGGCACCGAGTCG | | UGGCACCGAGUCGGUGC | | | |
| | GTGC | | | | | |
| 33 | TAATACGACTCACTATAGGCGAGGGCG | 281 | GCGAGGGCGAUGCCACCUAGUGUUUG | 282 | 95 | Cleavage |
| | ATGCCACCTAGTGTTTGAGCGCGAAAA | | AGCGCGAAAAAGCCAGACAUAAAUCG | | | Incapable |
| | AGCCAGACATAAATCGGCAAGTCGAAT | | GCAAGUCGAAUUUCAACCCGUAGCGG | | | Sequences |
| | TTCAACCCGTAGCGGTGGCACCGAGTC | | UGGCACCGAGUCGGUGC | | | |
| | GGTGC | | | | | |
| 34 | TAATACGACTCACTATAGGCGAGGGCG | 283 | GCGAGGGCGAUGCCACCUACACUCAC | 284 | 95 | Cleavage |
| | ATGCCACCTACACTCACAGCTAGAAAT | | AGCUAGAAAUAGCAAGUUGAAUUAA | | | Incapable |
| | AGCAAGTTGAATTAAGGGTAGTACGTG | | GGGUAGUACGUGAGCAACCUUCAUUG | | | Sequences |
| | AGCAACCTTCATTGGTGGCACCGAGTC | | GUGGCACCGAGUCGGUGC | | | |
| | GGTGC | | | | | |
| 35 | TAATACGACTCACTATAGGCGAGGGCG | 285 | GCGAGGGCGAUGCCACCUAGAGAUAG | 286 | 95 | Cleavage |
| | ATGCCACCTAGAGATAGTGCTCCAAAT | | UGCUCCAAAUGGGAGAUCACAAUCAA | | | Incapable |
| | GGGAGATCACAATCAAGTTAGTCGTTT | | GUUAGUCGUUUAUCAACCUGCAUGUG | | | Sequences |
| | ATCAACCTGCATGTGTGGCACCGAGTC | | UGGCACCGAGUCGGUGC | | | |
| | GGTGC | | | | | |
| 36 | TAATACGACTCACTATAGGCGAGGGCG | 287 | GCGAGGGCGAUGCCACCUAUUGUUCG | 288 | 95 | Cleavage |
| | ATGCCACCTATTGTTCGAGCTAGCATTA | | AGCUAGCAUUAGCAAGUGAAACUAGC | | | Incapable |
| | GCAAGTGAAACTAGCGATTAACCCTTCT | | GAUUAACCCUUCUUUCCUCCCACUGG | | | Sequences |
| | TTCCTCCCACTGGTGGCACCGAGTCGGT | | UGGCACCGAGUCGGUGC | | | |
| | GC | | | | | |
| 37 | TAATACGACTCACTATAGGCGAGGGCG | 289 | GCGAGGGCGAUGCCACCUAUGGCUGU | 290 | 95 | Cleavage |
| | ATGCCACCTATGGCTGTAGCAGGAAAT | | AGCAGGAAAUGGCUAAUGCAAGUUA | | | Incapable |
| | GGCTAATGCAAGTTAGGGTAGGCCGCG | | GGGUAGGCCGCGAGCAACUCGAACCG | | | Sequences |
| | AGCAACTCGAACCGCGGGCACCGAGTC | | CGGGCACCGAGUCGGUGC | | | |
| | GGTGC | | | | | |
| 41 | TAATACGACTCACTATAGGCGAGGGCG | 291 | GCGAGGGCGAUGCCACCUAUAUUUAC | 292 | 95 | Cleavage |
| | ATGCCACCTATATTTACAGCTGAGATTA | | AGCUGAGAUUAGCCAGUUAAAAUAA | | | Incapable |
| | GCCAGTTAAAATAAGGCTCAGTCCGTT | | GGCUCAGUCCGUUAUCAACUUUACCA | | | Sequences |
| | ATCAACTTTACCACGTGGCACCGAGTCG | | CGUGGCACCGAGUCGGUGC | | | |
| | GTGC | | | | | |
| 43 | TAATACGACTCACTATAGGCGAGGGCG | 293 | GCGAGGGCGAUGCCACCUAGCUCUGA | 294 | 95 | Cleavage |
| | ATGCCACCTAGCTCTGACCTTTGCAATA | | CCUUUGCAAUAUCGGCUUAAACUCAG | | | Incapable |
| | TCGGCTTAAACTCAGACGGGTGCGTTCT | | ACGGGUGCGUUCUUAACUGUAUUCGG | | | Sequences |
| | TAACTGTATTCGGTGGCACCGAGTCGGT | | UGGCACCGAGUCGGUGC | | | |
| | GC | | | | | |
| 45 | TAATACGACTCACTATAGGCGAGGGCG | 295 | GCGAGGGCGAUGCCACCUAGAUUUGU | 296 | 95 | Cleavage |
| | ATGCCACCTAGATTTGTAGCTAGAAAA | | AGCUAGAAAAAGCGAGUCAAAUGCAG | | | Incapable |
| | AGCGAGTCAAATGCAGGCTAGTCCGTT | | GCUAGUCCGUUAUCAACUUGACAUAG | | | Sequences |
| | ATCAACTTGACATAGTGGCACCGAGTC | | UGGCACCGAGUCGGUGC | | | |
| | GGTGC | | | | | |
| 46 | TAATACGACTCACTATAGGCGAGGGCG | 297 | GCGAGGGCGAUGCCACCUAGUUUUAG | 298 | 95 | Cleavage |
| | ATGCCACCTAGTTTTAGCGTTAGAAGTA | | CGUUAGAAGUAGCAAGUUAAAAUAA | | | Incapable |
| | GCAAGTTAAAATAAGGCCCGTCCATTA | | GGCCCGUCCAUUAUGAACUGGAACCA | | | Sequences |
| | TGAACTGGAACCAGTGGCACCGAGTCG | | GUGGCACCGAGUCGGUGC | | | |
| | GTGC | | | | | |
| 48 | TAATACGACTCACTATAGGCGAGGGCG | 299 | GCGAGGGCGAUGCCACCUAGUUUUGG | 300 | 95 | Cleavage |
| | ATGCCACCTAGTTTTGGAGCTAAATAAA | | AGCUAAAUAAAGCAAGUCAAAAUAA | | | Incapable |
| | GCAAGTCAAAATAAGGCGACCCCGTAA | | GGCGACCCCGUAAACAACUUUAGAAC | | | Sequences |
| | ACAACTTTAGAACGTGGCACCGAGTCG | | GUGGCACCGAGUCGGUGC | | | |
| | GTGC | | | | | |
| 49 | TAATACGACTCACTATAGGCGAGGGCG | 301 | GCGAGGGCGAUGCCACCUAGUUUUAG | 302 | 95 | Cleavage |
| | ATGCCACCTAGTTTTAGTGCTAGAAAAC | | UGCUAGAAAACGCAAGUUAAAAUAA | | | Incapable |
| | GCAAGTTAAAATAAGGTTAATCTTTCAA | | GGUUAAUCUUUCAACAACUCGAAAAU | | | Sequences |
| | CAACTCGAAAATGTGGCACCGAGTCGG | | GUGGCACCGAGUCGGUGC | | | |
| | TGC | | | | | |
| 50 | TAATACGACTCACTATAGGCGAGGGCG | 303 | GCGAGGGCGAUGCCACCUAGUUUUAG | 304 | 95 | Cleavage |
| | ATGCCACCTAGTTTTAGAGTAAGCAGTT | | AGUAAGCAGUUACAAGUUAAAAUAU | | | Incapable |
| | ACAAGTTAAAATATGGCTGGTCCGTTAT | | GGCUGGUCCGUUAUCAACUGUGAAAC | | | Sequences |
| | CAACTGTGAAACGTGGCACCGAGTCGG | | GUGGCACCGAGUCGGUGC | | | |
| | TGC | | | | | |
| 51 | TAATACGACTCACTATAGGCGAGGGCG | 305 | GCGAGGGCGAUGCCACCUAGUUAAGC | 306 | 95 | Cleavage |
| | ATGCCACCTAGTTAAGCCGTCAAAAATT | | CGUCAAAAAUUUGAGCUUACGAAAUG | | | Incapable |
| | TGAGCTTACGAAATGGCAAGTCGCTTAT | | GCAAGUCGCUUAUCAACCCAAAUCGG | | | Sequences |
| | CAACCCAAATCGGTGGCACCGAGTCGG | | UGGCACCGAGUCGGUGC | | | |
| | TGC | | | | | |
| 52 | TAATACGACTCACTATAGGCGAGGGCG | 307 | GCGAGGGCGAUGCCACCUAGUUGUAA | 308 | 95 | Cleavage |
| | ATGCCACCTAGTTGTAACGGTGGAAAC | | CGGUGGAAACGUCGACUUAAUAUUGU | | | Incapable |
| | GTCGACTTAATATTGTGCTCGTCAGTGA | | GCUCGUCAGUGAUCAACUUAUCGGUG | | | Sequences |
| | TCAACTTATCGGTGTGGCACCGAGTCGG | | UGGCACCGAGUCGGUGC | | | |
| | TGC | | | | | |
| 53 | TAATACGACTCACTATAGGCGAGGGCG | 309 | GCGAGGGCGAUGCCACCUAGUUUCUU | 310 | 95 | Cleavage |
| | ATGCCACCTAGTTTCTTAACCCGAAAGT | | AACCCGAAAGUUAAAGUCAAACUAAG | | | Incapable |
| | TAAAGTCAAACTAAGTCTTGTGAGTTCG | | UCUUGUGAGUUCGCCAUUUGAACUGG | | | Sequences |
| | CCATTTGAACTGGTGGCACCGAGTCGGT | | UGGCACCGAGUCGGUGC | | | |
| | GC | | | | | |
| 54 | TAATACGACTCACTATAGGCGAGGGCG | 311 | GCGAGGGCGAUGCCACCUAGUUUUAG | 312 | 95 | Cleavage |
| | ATGCCACCTAGTTTTAGAGCCTCCACTG | | AGCCUCCACUGGCAAGUUAAAAUAAG | | | Incapable |
| | GCAAGTTAAAATAAGACTAGTCCGTTA | | ACUAGUCCGUUAUCAACUUGACAACG | | | Sequences |
| | TCAACTTGACAACGTGGCACCGAGTCG | | UGGCACCGAGUCGGUGC | | | |
| | GTGC | | | | | |
| 55 | TAATACGACTCACTATAGGCGAGGGCG | 313 | GCGAGGGCGAUGCCACCUAGUUAGAG | 314 | 95 | Cleavage |
| | ATGCCACCTAGTTAGAGTGCATTGAATC | | UGCAUUGAAUCGCCAGUGAAAUUAAG | | | Incapable |
| | GCCAGTGAAATTAAGGCTAGTCCGTTAT | | GCUAGUCCGUUAUCAACAUCAACCUG | | | Sequences |
| | CAACATCAACCTGTGGCACCGAGTCGG | | UGGCACCGAGUCGGUGC | | | |
| | TGC | | | | | |
| 56 | TAATACGACTCACTATAGGCGAGGGCG | 315 | GCGAGGGCGAUGCCACCUAGAUUUAG | 316 | 95 | Cleavage |
| | ATGCCACCTAGATTTAGAGCTAGCATTA | | AGCUAGCAUUAGCAAGUUAAAACAAA | | | Incapable |
| | GCAAGTTAAAACAAAGGTGTTGCACTC | | GGUGUUGCACUCCAUACUUGAGGAUG | | | Sequences |
| | CATACTTGAGGATGTGGCACCGAGTCG | | UGGCACCGAGUCGGUGC | | | |
| | GTGC | | | | | |
| 57 | TAATACGACTCACTATAGGCGAGGGCG | 317 | GCGAGGGCGAUGCCACCUAGUUUUCG | 318 | 95 | Cleavage |
| | ATGCCACCTAGTTTTCGCCTTAGAAATT | | CCUUAGAAAUUGCCACGUAAAAUUAA | | | Incapable |
| | GCCACGTAAAATTAACCTAGTCCGTTAT | | CCUAGUCCGUUAUCAACGUGUAACCG | | | Sequences |
| | CAACGTGTAACCGTGGCACCGAGTCGG | | UGGCACCGAGUCGGUGC | | | |
| | TGC | | | | | |
| 58 | TAATACGACTCACTATAGGCGAGGGCG | 319 | GCGAGGGCGAUGCCACCUAAAGCUAA | 320 | 95 | Cleavage |
| | ATGCCACCTAAAGCTAAAGCTTGAAAG | | AGCUUGAAAGAGCUAACUACAACUUG | | | Incapable |
| | AGCTAACTACAACTTGCCCTGTTGGGTA | | CCCUGUUGGGUAUCACCAUGACCAUG | | | Sequences |
| | TCACCATGACCATGGGGCACCGAGTCG | | GGGCACCGAGUCGGUGC | | | |
| | GTGC | | | | | |
| 59 | TAATACGACTCACTATAGGCGAGGGCG | 321 | GCGAGGGCGAUGCCACCUAGACUUAG | 322 | 95 | Cleavage |
| | ATGCCACCTAGACTTAGAGCTTATAATA | | AGCUUAUAAUAGAAAGCUACUAUUA | | | Incapable |
| | GAAAGCTACTATTAGGAAACATCATGA | | GGAAACAUCAUGACCCACGUGCCAUG | | | Sequences |
| | CCCACGTGCCATGGTGGCACCGAGTCG | | GUGGCACCGAGUCGGUGC | | | |
| | GTGC | | | | | |
| 60 | TAATACGACTCACTATAGGCGAGGGCG | 323 | GCGAGGGCGAUGCCACCUAGUUUUAG | 324 | 95 | Cleavage |
| | ATGCCACCTAGTTTTAGAGCGAAAACTT | | AGCGAAAACUUCCCAGUUAAAACAAG | | | Incapable |
| | CCCAGTTAAAACAAGGCAAGTCCGTTA | | GCAAGUCCGUUAUCAACUGUAACAGU | | | Sequences |
| | TCAACTGTAACAGTGGCACCGAGTCGG | | GGCACCGAGUCGGUGC | | | |
| | TGC | | | | | |
| 1388 | TAATACGACTCACTATAGGCGAGGGCG | 325 | GCGAGGGCGAUGCCACCUAGGUUUAG | 326 | 95 | Cleavage |
| | ATGCCACCTAGGTTTAGCGCTGTGAACA | | CGCUGUGAACAGCAAUUGAAACUAAA | | | Incapable |
| | GCAATTGAAACTAAACTTAGTCGGTGA | | CUUAGUCGGUGACCAACUUGAACGUG | | | Sequences |
| | CCAACTTGAACGTGGGGCACCGAGTCG | | GGGCACCGAGUCGGUGC | | | |
| | GTGC | | | | | |
| 3640 | TAATACGACTCACTATAGGCGAGGGCG | 327 | GCGAGGGCGAUGCCACCUAAUUUUAG | 328 | 95 | Cleavage |
| | ATGCCACCTAATTTTAGTGCTAGAATTA | | UGCUAGAAUUAGCAAGUUAAAAUUC | | | Incapable |
| | GCAAGTTAAAATTCGGTGACACCCTGCT | | GGUGACACCCUGCUCAUCUUGCAGGC | | | Sequences |
| | CATCTTGCAGGCGGGGCACCGAGTCGG | | GGGGCACCGAGUCGGUGC | | | |
| | TGC | | | | | |
| 4237 | TAATACGACTCACTATAGGCGAGGGCG | 329 | GCGAGGGCGAUGCCACCUAGUUUUAG | 330 | 95 | Cleavage |
| | ATGCCACCTAGTTTTAGTGCTAGAAATA | | UGCUAGAAAUAGCAAGUUAAAAUUG | | | Incapable |
| | GCAAGTTAAAATTGGTCCAGTCCATGTG | | GUCCAGUCCAUGUGCCACGUGAACAU | | | Sequences |
| | CCACGTGAACATGTGGCACCGAGTCGG | | GUGGCACCGAGUCGGUGC | | | |
| | TGC | | | | | |
| 6714 | TAATACGACTCACTATAGGCGAGGGCG | 331 | GCGAGGGCGAUGCCACCUAGUGUUAC | 332 | 95 | Cleavage |
| | ATGCCACCTAGTGTTACCACTAGACTTA | | CACUAGACUUAACAAGUGAAAGUAAU | | | Incapable |
| | ACAAGTGAAAGTAATTCGAGTTTGTTAC | | UCGAGUUUGUUACCGGUCCGUAACGG | | | Sequences |
| | CGGTCCGTAACGGTGGCACCGAGTCGG | | UGGCACCGAGUCGGUGC | | | |
| | TGC | | | | | |
| 18581 | TAATACGACTCACTATAGGCGAGGGCG | 333 | GCGAGGGCGAUGCCACCUAGCUUUAC | 334 | 95 | Cleavage |
| | ATGCCACCTAGCTTTACCGCGAGAGAT | | CGCGAGAGAUAGCAAGUUAAAAUACG | | | Incapable |
| | AGCAAGTTAAAATACGCTACGTACGGT | | CUACGUACGGUUGCUAUGUGACAACG | | | Sequences |
| | TGCTATGTGACAACGTGGCACCGAGTC | | UGGCACCGAGUCGGUGC | | | |
| | GGTGC | | | | | |
| 26747 | TAATACGACTCACTATAGGCGAGGGCG | 335 | GCGAGGGCGAUGCCACCUAUUUUUAG | 336 | 95 | Cleavage |
| | ATGCCACCTATTTTTAGCGCTAGAACTA | | CGCUAGAACUAGCUCGUGUAAAAAUU | | | Incapable |
| | GCTCGTGTAAAAATTCCTAGTACGTTAT | | CCUAGUACGUUAUCAACUUAAUCGAG | | | Sequences |
| | CAACTTAATCGAGTGGCACCGAGTCGG | | UGGCACCGAGUCGGUGC | | | |
| | TGC | | | | | |
| 29321 | TAATACGACTCACTATAGGCGAGGGCG | 337 | GCGAGGGCGAUGCCACCUAGUAUUCG | 338 | 95 | Cleavage |
| | ATGCCACCTAGTATTCGAGCTAGAAAT | | AGCUAGAAAUAGCAAGUGAAUACAA | | | Incapable |
| | AGCAAGTGAATACAAGGCTAATCCGTT | | GGCUAAUCCGUUAUCAACACGCCCCG | | | Sequences |
| | ATCAACACGCCCCGGTGGCACCGAGTC | | GUGGCACCGAGUCGGUGC | | | |
| | GGTGC | | | | | |
| 39145 | TAATACGACTCACTATAGGCGAGGGCG | 339 | GCGAGGGCGAUGCCACCUAGUUUUCG | 340 | 95 | Cleavage |
| | ATGCCACCTAGTTTTCGAGCTAGAAATA | | AGCUAGAAAUAGUAUGUGAAAAAUC | | | Incapable |
| | GTATGTGAAAAATCGGCTAGTACGGTA | | GGCUAGUACGGUAUCUACGUUAAGUA | | | Sequences |
| | TCTACGTTAAGTAGTGGCACCGAGTCG | | GUGGCACCGAGUCGGUGC | | | |
| | GTGC | | | | | |
| 45715 | TAATACGACTCACTATAGGCGAGGGCG | 341 | GCGAGGGCGAUGCCACCUAGUUUUAG | 342 | 95 | Cleavage |
| | ATGCCACCTAGTTTTAGAGCTAGTAATA | | AGCUAGUAAUAGCCAGUUAAAAUAA | | | Incapable |
| | GCCAGTTAAAATAAGTCTGTTCCGTAAT | | GUCUGUUCCGUAAUCCACAUGAUUAC | | | Sequences |
| | CCACATGATTACGTGGCACCGAGTCGG | | GUGGCACCGAGUCGGUGC | | | |
| | TGC | | | | | |
| 45875 | TAATACGACTCACTATAGGCGAGGGCG | 343 | GCGAGGGCGAUGCCACCUAGUUUUAC | 344 | 95 | Cleavage |
| | ATGCCACCTAGTTTTACAGATTGACATA | | AGAUUGACAUAGCAAGUUAAAACACU | | | Incapable |
| | GCAAGTTAAAACACTGCACGCCCGTTCT | | GCACGCCCGUUCUCGACUUGUAAACG | | | Sequences |
| | CGACTTGTAAACGTGGCACCGAGTCGG | | UGGCACCGAGUCGGUGC | | | |
| | TGC | | | | | |

### Example 2—Evolution of Functional CRISPR Guide RNA Variants to Facilitate High Resolution Editing

CRISPR-based editing has revolutionized genome engineering despite the observation that many DNA sequences remain challenging to target. Unproductive interactions formed between the guide (g)RNAs' functional domains, Cas9-binding aptamer-scaffold domain and DNA-binding antisense domain, are often responsible for such limited editing resolution. The inventors utilize SELEX (Systematic Evolution of Ligands by Exponential Enrichment) to identify numerous aptamer variants that bind Cas9 and support efficient DNA cleavage. The inventors observe that particular Cas9-binding aptamer domains pair most effectively with particular DNA-binding antisense domains, yielding gRNA combinations with enhanced editing efficiencies at various sites. These results indicate that by expanding the repertoire of functional gRNA aptamer-scaffold domains, CRISPR-based systems can be created to efficiently target additional DNA sequences and thereby greatly expand the repertoire of genomic sites tractable to editing.

The discovery that a CRISPR-based guide (g)RNA can be programmed to bind and deliver a Cas9 protein, with a nuclease or other editing activity, to a particular DNA sequence has revolutionized genome engineering (1-6). Unfortunately many DNA sites remain challenging to target for editing despite the development of improved targeting rules and efforts to rationally modify gRNAs to attempt to increase their ability to support editing activity(?-9). To enable editing, the gRNA must permit two RNA domains to fold and function in concert: a Cas9-binding aptamer domain that serves as a scaffold to bind and position the Cas9 protein for editing and a DNA-binding antisense domain composed of an RNA sequence complementary to the genomic target sequence of interest. See FIG. 21. Unfortunately, it is well established that the proper folding of aptamers can be adversely affected by their flanking sequences which can greatly limit their activities (7, 10).

To facilitate proper RNA aptamer folding in the context of flanking sequences for gene therapy applications, the inventors have previously explored the use of high through-put screening of large RNA libraries via expression cassette SELEX (Systematic Evolution of Ligands by EXponential enrichment) (10-12). Here the sequences immediately adjacent to the aptamer are randomized and flanking sequences that allowed for proper aptamer folding were isolated through their ability to bind the target protein with high affinity (10). Unfortunately, this approach is not amenable to Cas9 aptamer evolution as its 5′ flanking RNA sequence is dictated by the DNA sequence being targeted. Moreover, this flanking “DNA-binding” sequence needs to be changeable to match each new genomic target of interest. Therefore, the inventors decided to explore an alternative approach and ask if SELEX could be utilized to isolate alternative gRNA-aptamer domains that can fold into active conformations in the context of a fixed flanking sequence. Our studies reveal that the gRNA aptamer domain is quite malleable. This flexibility allowed for the identification of numerous functional gRNA-aptamer variants that can be paired with particular DNA targeting domains to generate full-length gRNAs that are effective against different DNA target sites. The ability to utilize high through-put screening and RNA aptamer evolution to generate optimized gRNAs for CRISPR-based editing agents promises to dramatically expand the DNA target sites that are amenable to efficient and specific editing.

### Results

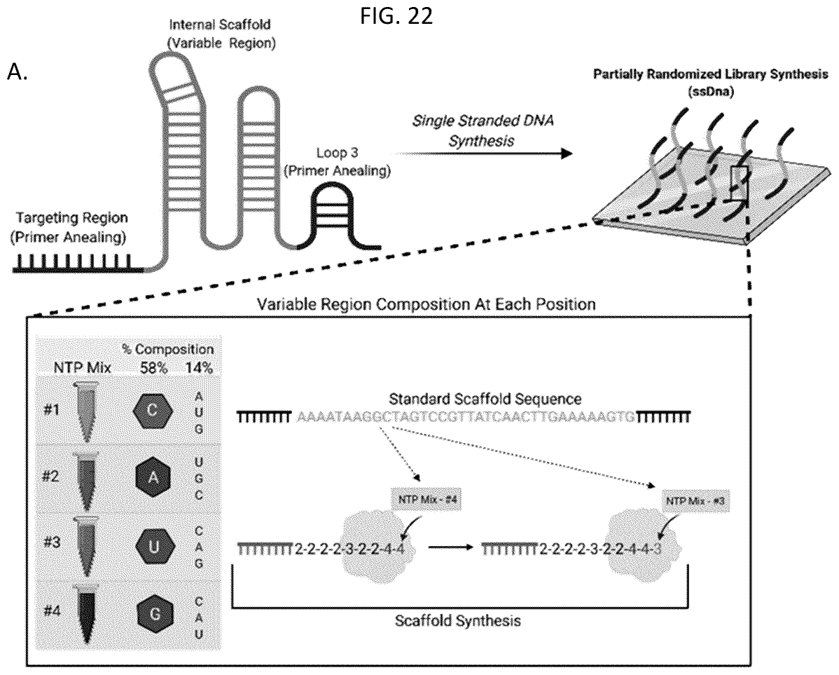

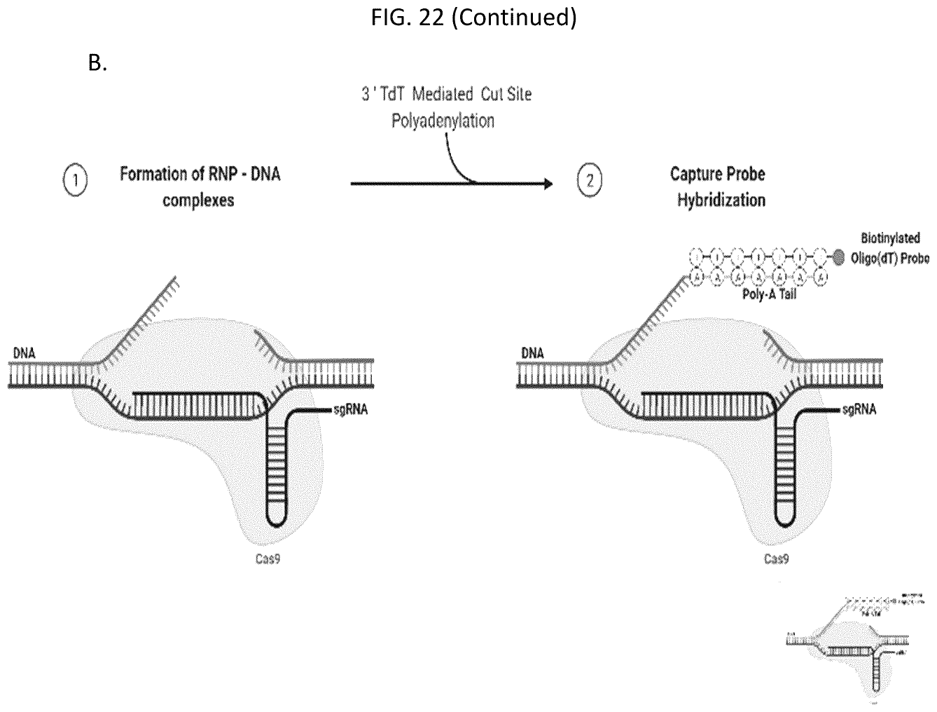

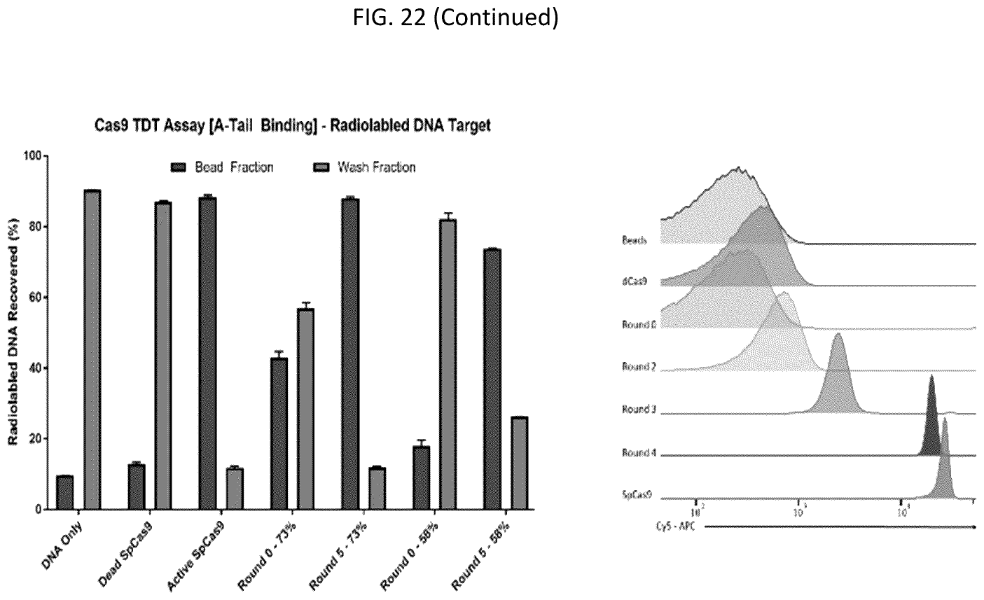

To examine the mutational landscape functionally tolerated by the aptamer portion of the Streptococcus pyogenes SpCas9 (single guide) sgRNA (1), the inventors generated a partially randomized gRNA library biased towards the wild-type (WT) aptamer-scaffold. The 5′ 20-nt DNA targeting region, directed towards a sequence in the GFP gene, and stem-loop 3 of the gRNA were utilized for library amplification and remained constant during the selection (FIG. 22a). The 60 nucleotides comprising the repeat and anti-repeat regions, stem-loop 1 and stem-loop 2 of the gRNA were randomized (FIG. 22a). To generate significant variation for selection, an aptamer gRNA library containing 58% of the wild type nucleotide at each of the 60 positions and 42% of the other three nucleotides present in equal amounts was created (FIG. 22a).

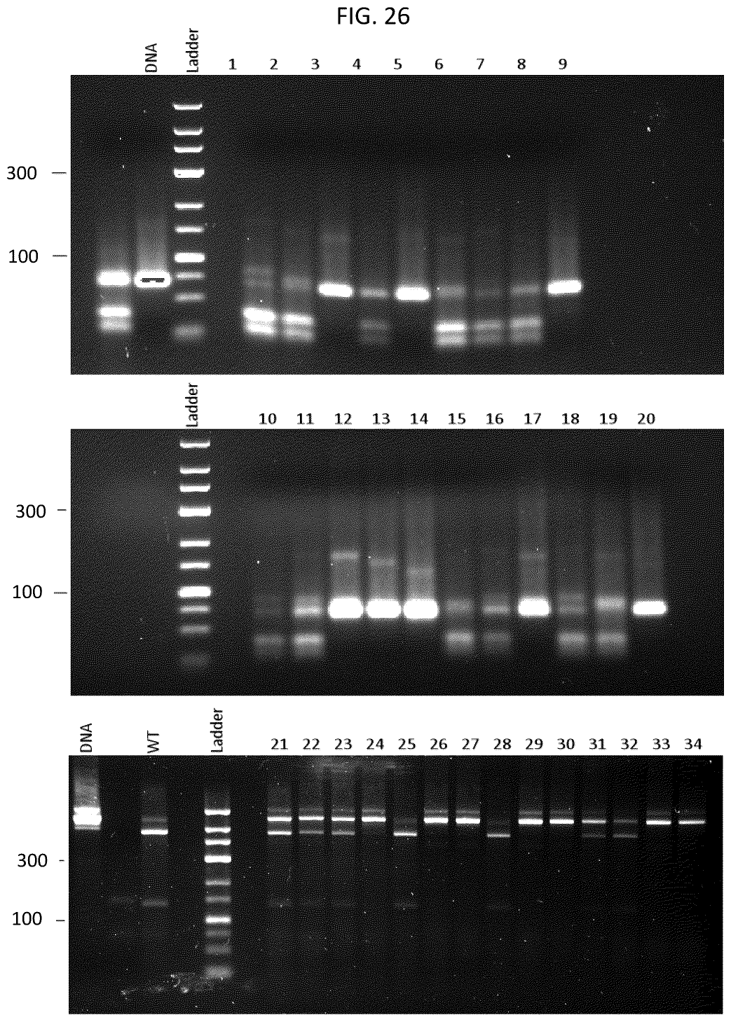

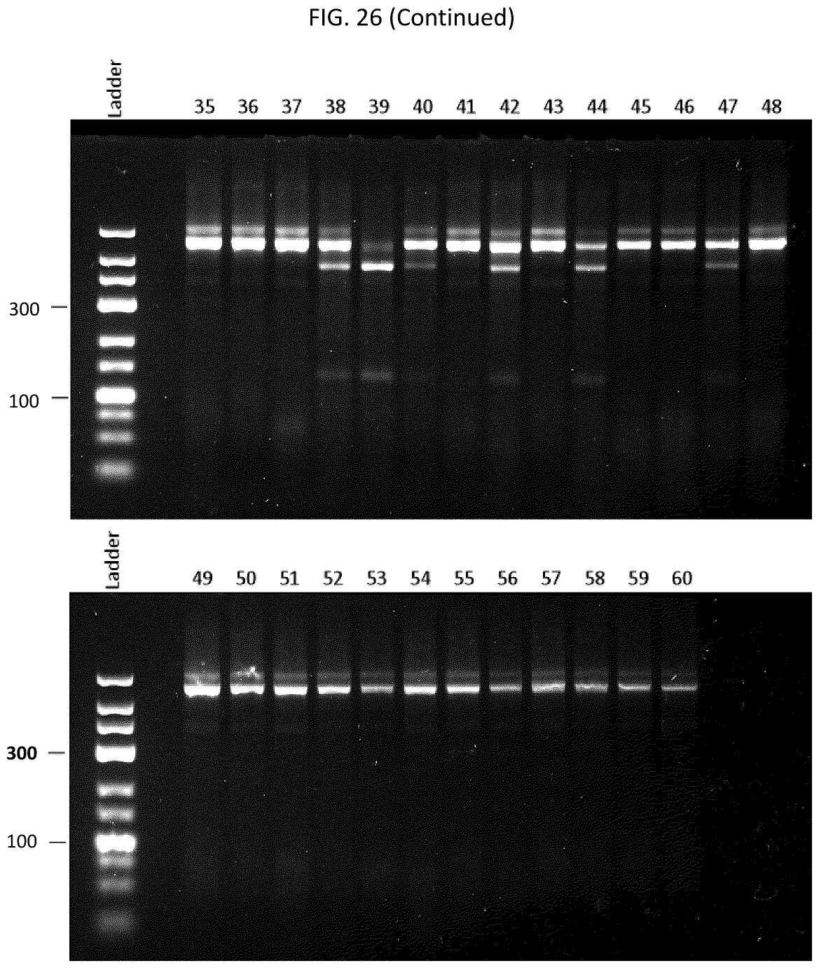

To isolate sgRNA variants that could support SpCas9-mediated cleavage of DNA from this library of >10¹⁴ variants, the inventors selected for both ribonucleoprotein complex (RNP) formation and DNA cleavage (FIG. 22b). Streptavidin-bead-based SELEX was used to isolate sgRNA variants capable of binding SpCas9 (11-15). Briefly, sgRNA libraries were incubated with decreasing concentrations of recombinant SpCas9. gRNA variants that bound to the protein were partitioned from unbound RNAs, reverse transcribed, amplified by PCR and then transcribed for an additional round of selection. After performing initial rounds of SELEX, the inventors analyzed the resulting gRNA clones and determined that very few were able to support DNA cleavage. To isolate those gRNA variants capable of binding and supporting Cas9-mediated DNA cleavage, a functional screen was added to the SELEX approach. Following cleavage by SpCas9, the 3′ end of the PAM-distal non-target strand is released from the Cas9-DNA complex and is available for enzymatic modification without complex dissociation (16). Terminal deoxynucleotidyl transferase (TdT), an enzyme that catalyzes the addition of nucleotides to the 3′ hydroxyl terminus, can then be used in an A-tailing reaction to extend the free 3′ end of the PAM-distal non-target DNA strand (FIG. 6 and FIG. 22B(2)). The cleaved Cas9-DNA complexes can then be isolated using a biotinylated Oligo(dT) to capture the reaction products, enriching for gRNA variants that can form cleavage competent Cas9 RNPs (FIG. 22c). Using this TdT approach with radiolabeled target DNA, the inventors observed a 3-fold increase in enrichment of cleavage competent SpCas9 RNP complexes when compared to dCas9 RNPs (FIG. 22c). Following validation, the TdT partitioning approach was implemented for all subsequent rounds of selection. To evaluate the progress of the selection, the target DNA was synthesized with a cy5 probe, enabling for rapid assessment of cleavage activity of all rounds by assessing the amount of the probe that was cleaved off a column (FIG. 22D, FIG. 26). Once the gRNA variants present in a particular round approached a level of cleavage activity comparable to the wild type gRNA scaffold (Round 4, FIGS. 22C and 22D), the resulting gRNAs variants were sequenced and analyzed for activity.

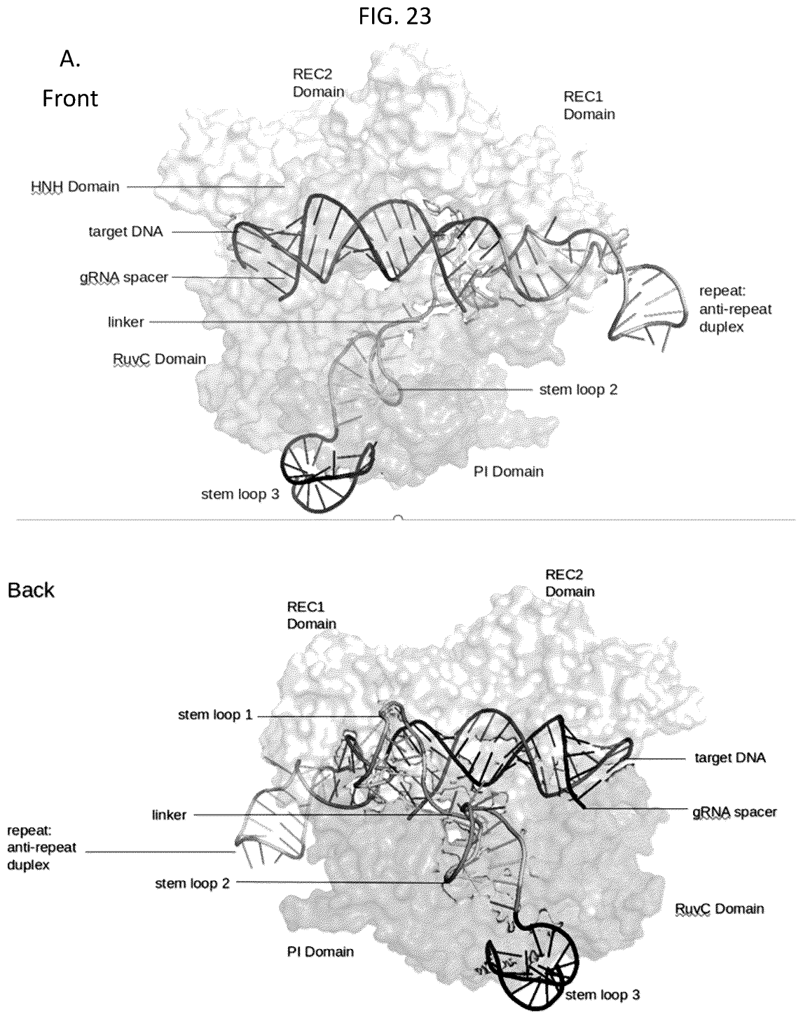

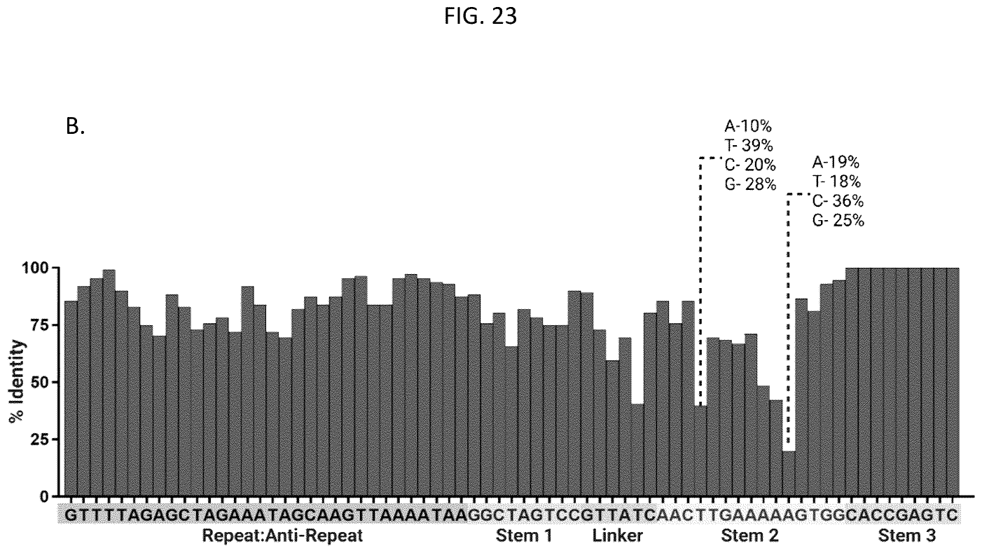

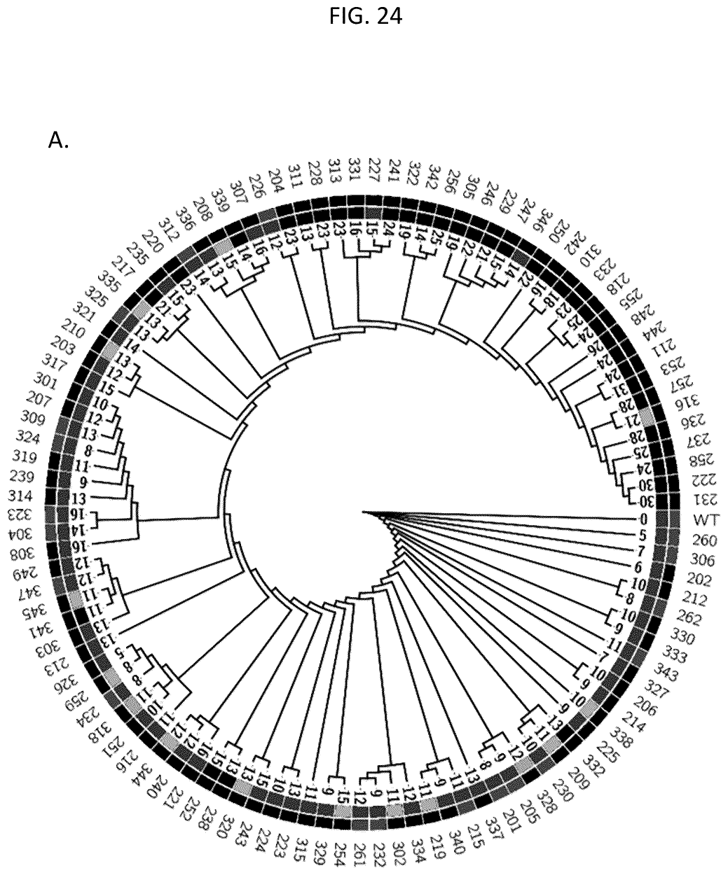

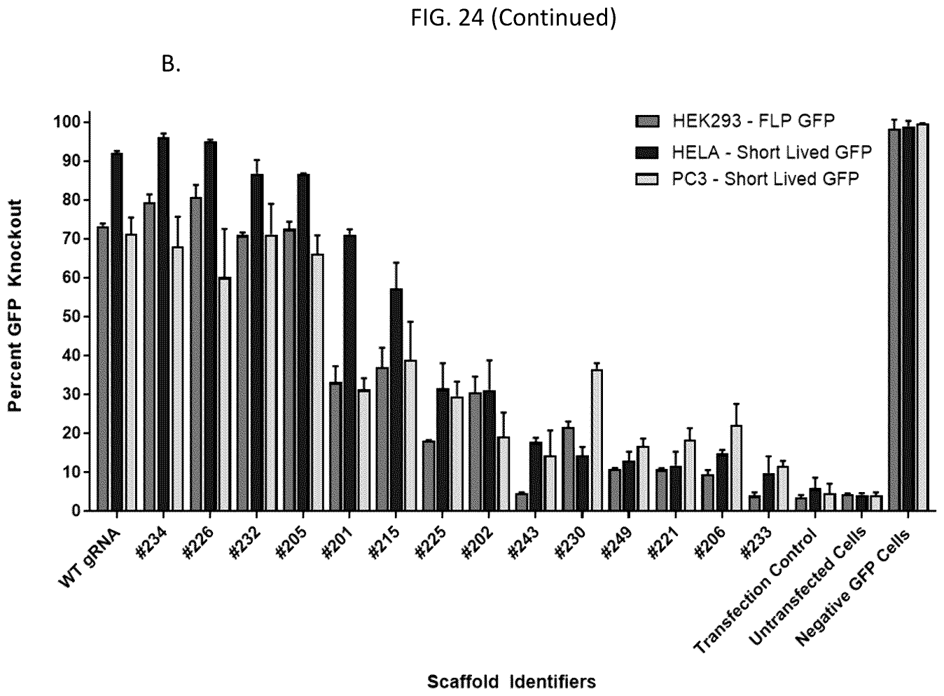

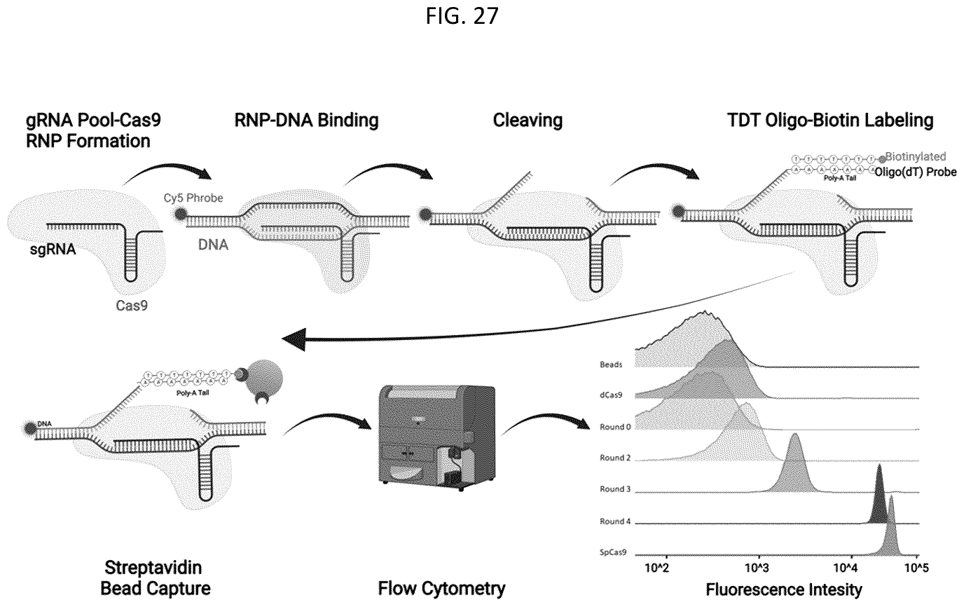

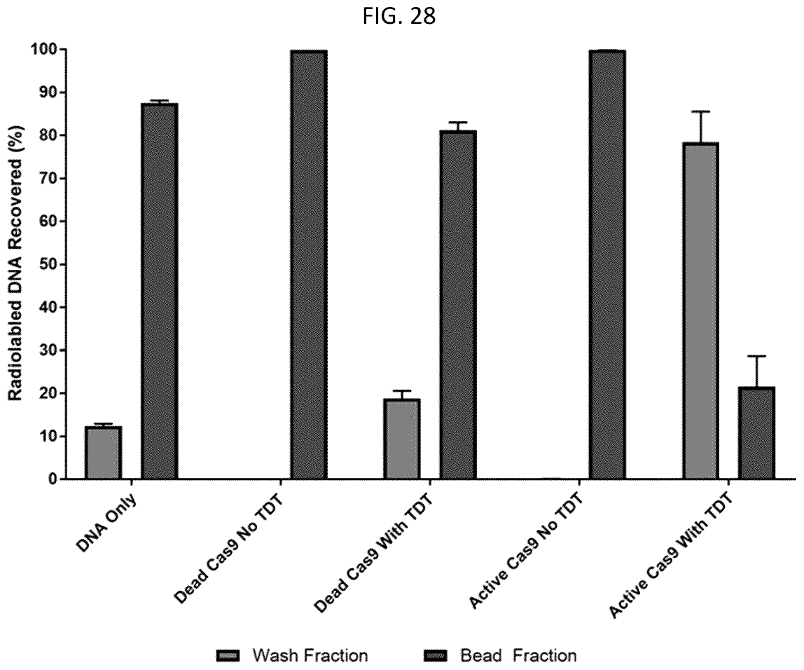

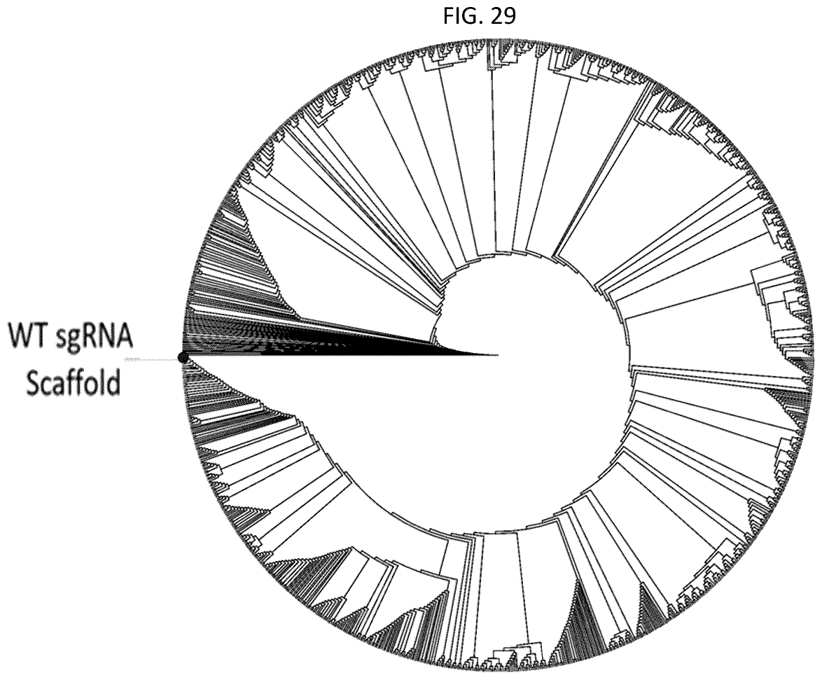

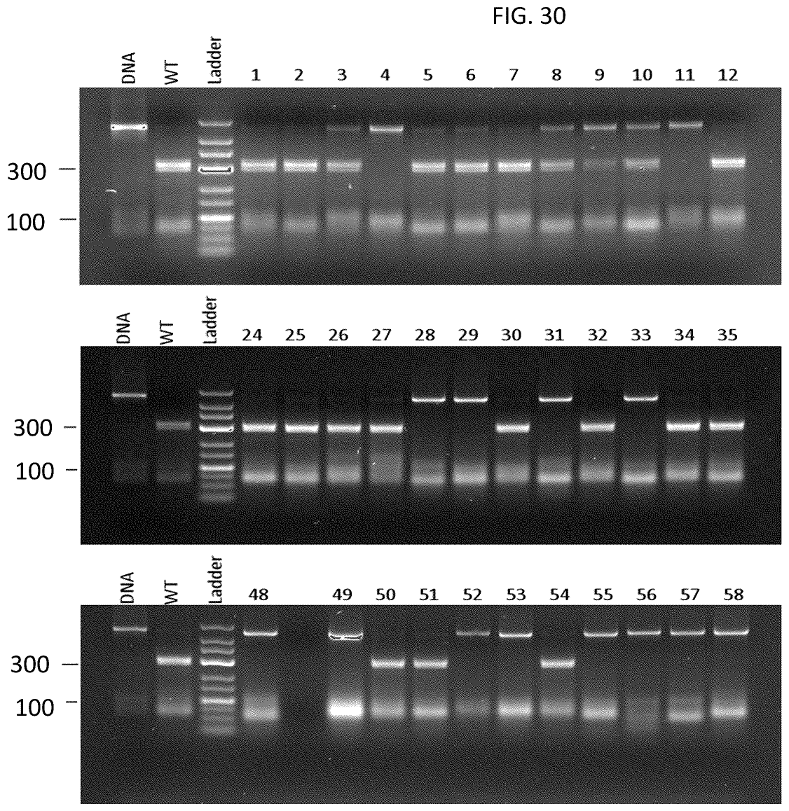

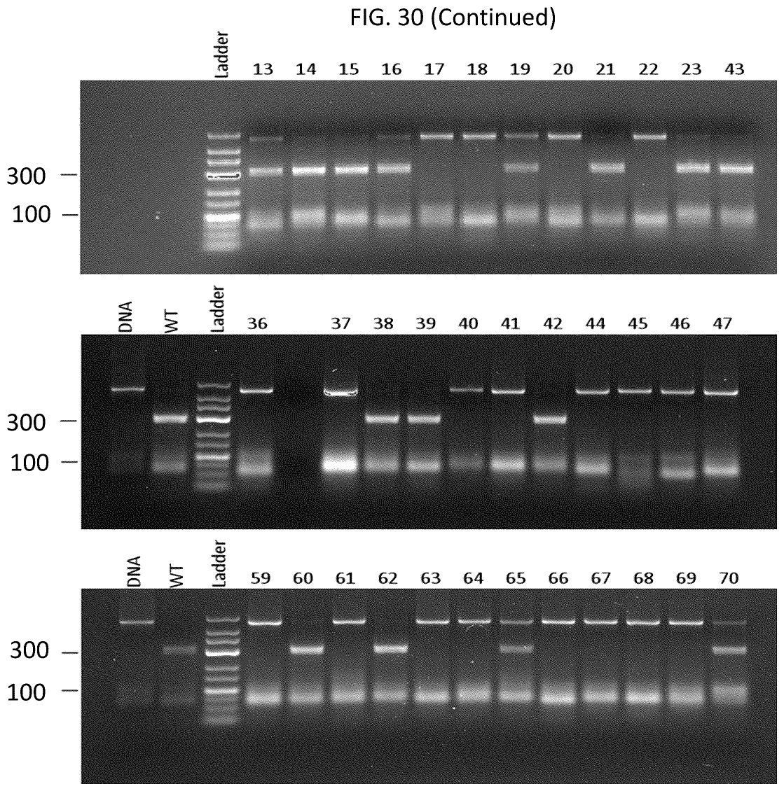

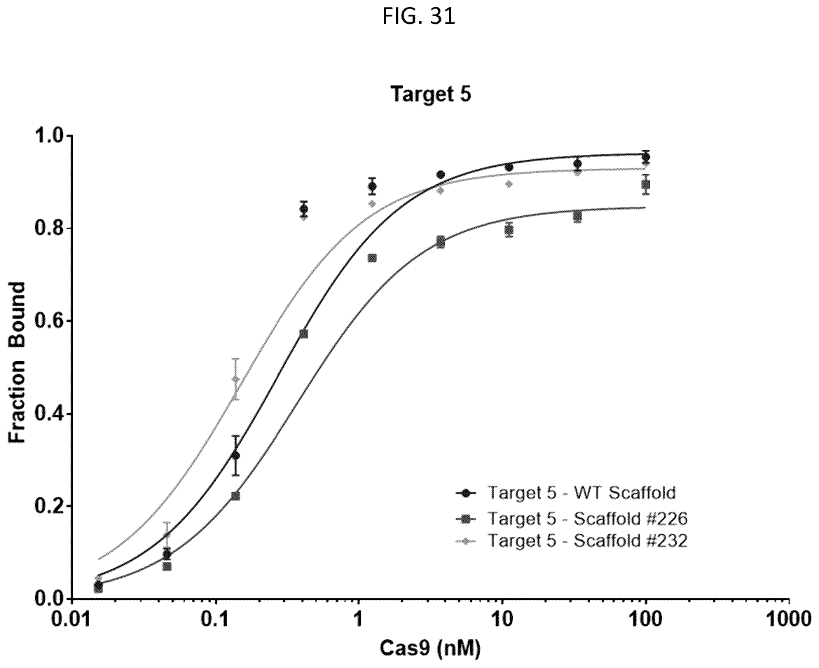

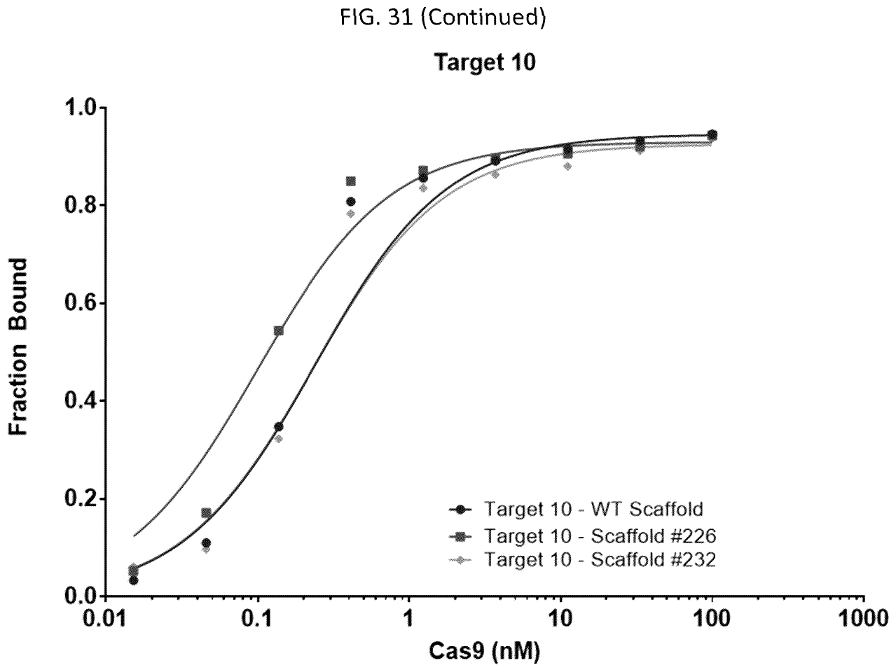

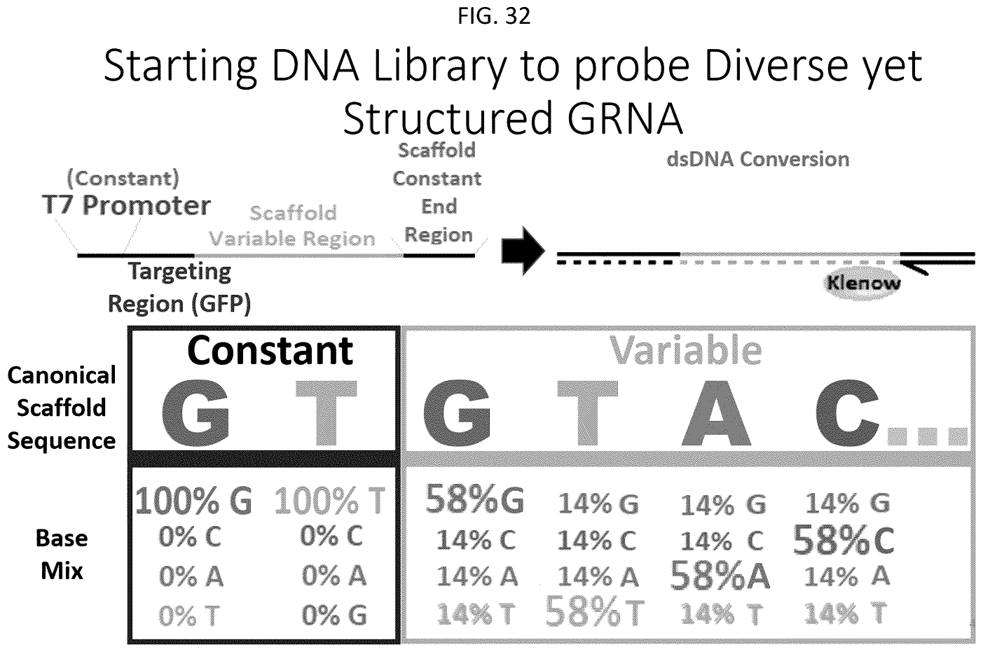

Sequencing of the round 5 pool of gRNAs yielded over 30,000 different gRNA aptamer variants (FIGS. 23 and 27). Cas9 in complex with the gRNAs is shown in FIG. 23. This large, diverse set of sgRNAs were organized by frequency of occurrence, of which, the top 2,000 sequences were grouped phylogenetically (FIGS. 24A and 28). Two hundred gRNA variants, representing various nodes of this phylogenetic tree containing diverse mutational profiles, were tested for their ability to support Cas9 cleavage of DNA and to ascertain how well the gRNA can tolerate sequence changes in its aptamer domain and still enable SpCas9-mediated cleavage. Remarkably, 109 of these 200 gRNA variants supported efficient DNA cleavage in vitro, the majority of which cleaved the target DNA to near completion in 30 minutes, similarly to the wild type gRNA scaffold (FIG. 23b & FIG. 26). Some of these aptamer domains tolerated up to 20 nucleotide alterations out of 60 positions and still supported in vitro cleavage. Regions that formed complementary base pairs within the scaffold secondary structure, such as at the beginning and end of the repeat: anti-repeat sequences, tended to remain unchanged when compared to the wild-type sgRNA sequence. Conversely, stem loop 1 and stem loop 2 tolerated a wide array of nucleotide changes that still facilitate cleavage activity (FIG. 23b). However, these results also indicate that certain positions within the sgRNA aptamer domain are difficult to alter and suggest that they are very important for cleavage activity. Interestingly, our results also indicate that a few specific nucleotides in the wild type gRNA are not preferred at least in the context of the GFP targeting sequence employed for the selection (FIG. 23b). Thus, the aptamer portion of the sgRNA tolerates numerous and multiple changes yet still maintains high affinity SpCas9 binding and supports efficient DNA cleavage.

Next the set of gRNAs capable of supporting in vitro DNA cleavage were tested for editing activity in mammalian cells. The inventors observed that approximately ⅓^{rd} of these gRNA variants retained activity in cells, defined as >20% editing efficiency, as measured by targeting the GFP gene sequence utilized during gRNA selection and assaying for loss of GFP expression following treatment of cells with the various gRNA-Cas9 RNPs (FIG. 24B). However, the inventors observed that certain gRNA aptamer variants were much more effective at editing the GFP gene than others and only a few were as efficient as the wild type gRNA for targeting this DNA sequence and knocking out GFP expression. Given that the inventors intentionally chose a GFP DNA target sequence that was known to be an efficient site for editing by the wild type gRNA, this result was not surprising; however it was notable that several gRNA variants with 8 to 12 nucleotide changes in their aptamer domains were also very effective (e.g. gRNAs 226 and 232) against this highly permissive wt gRNA-GFP editing site. To further characterize this subset of gRNA variants, the inventors evaluated six of them in additional cell lines and observed that they retained high levels of activity in multiple cell types. As shown in FIG. 24, all six retain activity in all three cell lines and in some cases are at least as effective at editing the GFP gene as the wt gRNA.

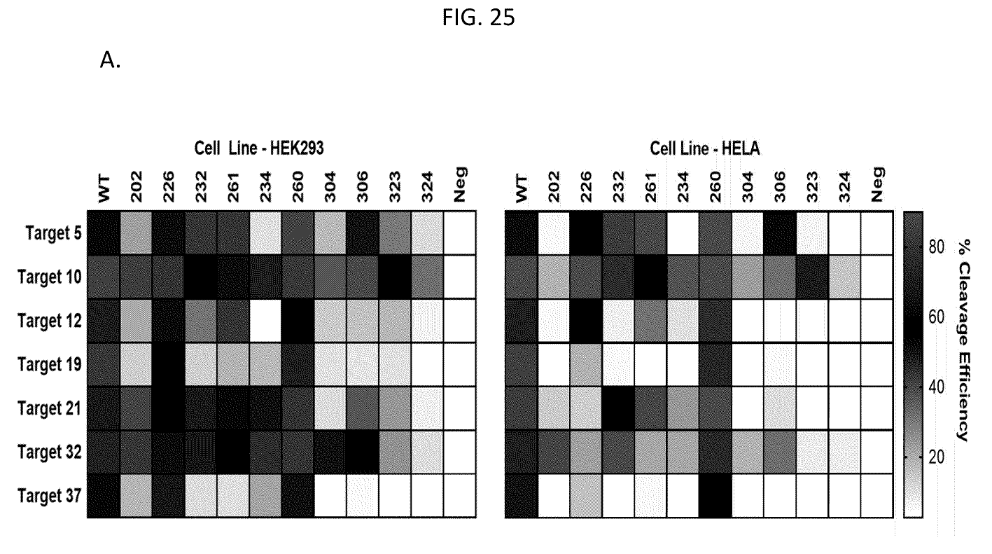

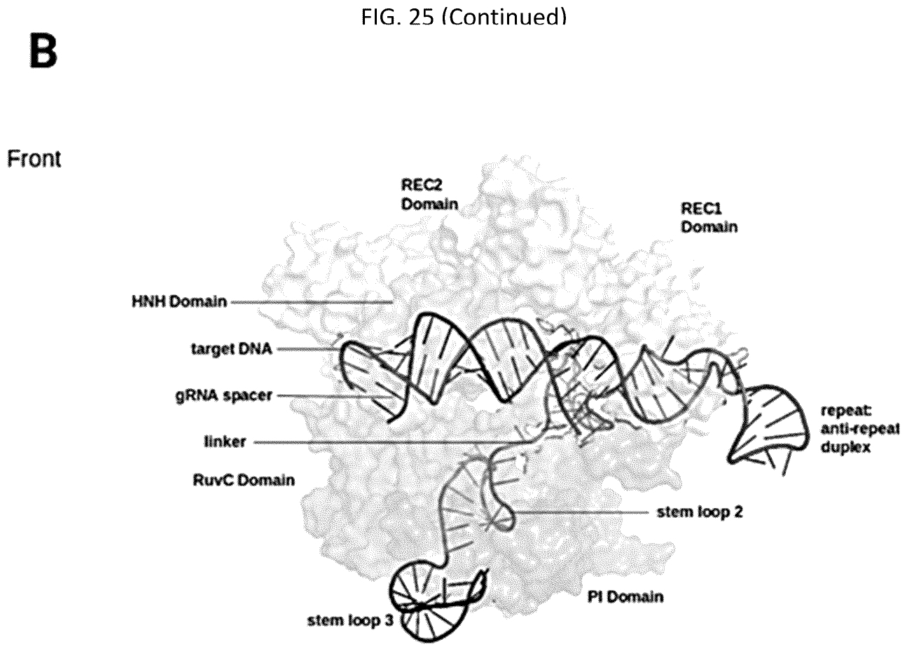

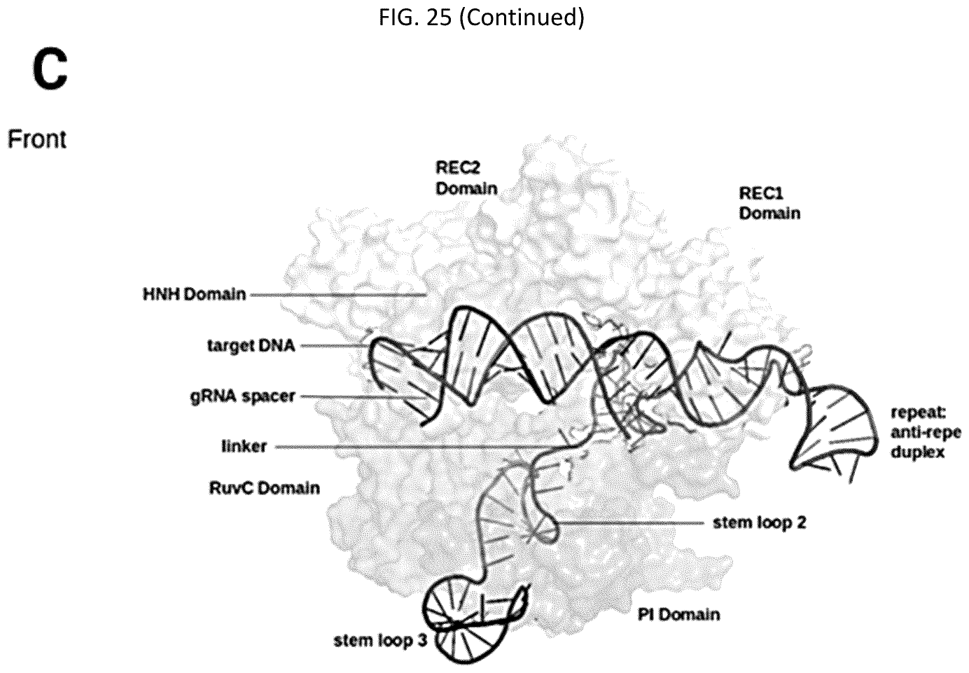

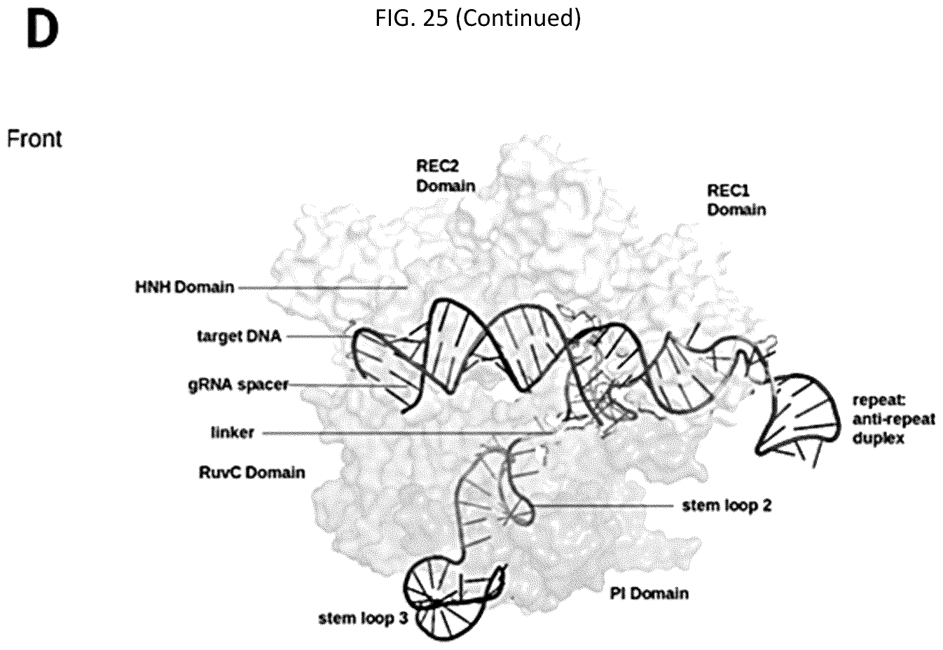

The observation that the gRNA can tolerate multiple nucleotide changes in its Cas9-binding aptamer domain led us to explore if different combinations of aptamer and DNA targeting domains might yield gRNAs with improved or reduced editing efficiencies at other DNA sites. Therefore the 20-nucleotide targeting region was changed to recognize five new PAM containing sites found in the GFP gene and these five DNA targeting domains were each paired with ten different aptamer domain variants that had emerged from the functional selection. These 50 GFP-targeting gRNAs were complexed with Cas9 and evaluated for their ability to edit the GFP gene in different Cell lines. As shown in FIG. 25, the different aptamer domains partner with the various DNA targeting domains to yield gRNAs with a wide range of editing abilities. However, for three out of five DNA target sites evaluated, combinations are identified that are more effective at editing than the wild type gRNA. Surprisingly, the inventors found that certain aptamer domains partner with particular DNA targeting domains more effectively than they do with the DNA targeting domain present in the gRNA library employed for selection. Interestingly one variant, gRNA260, pairs particularly well with three of the five targeting sequences. Thus, analysis of these 50 gRNA variants for editing activity indicates that the ten tested aptamer domains prefer different sets of DNA targeting domains and that some variants appear to be generalists, able to partner well with multiple DNA binding domains, while others are more selective and tend to partner best with a particular DNA binding domain. Highlighting the importance of the structure-function relationship required between their DNA-binding and Cas9-binding domains, the various gRNAs alter different sets of nucleotides in their aptamer domains to result in such a diverse range of targeting properties (FIG. 25 B-D).

These results indicate that functional in vitro selection and evolution, from a vast RNA library, can generate numerous sgRNAs variants that support SpCas9-mediated cleavage of DNA. Such variants have a range of distinct activities including the ability to target certain DNA sites more effectively than the wild type gRNA. This high-throughput gRNA selection approach can be utilized to optimize the targeting of any DNA sequence containing a SpCas9 PAM sequence which should greatly expand the repertoire of DNA sites amenable to efficient editing. Moreover by utilizing Toggle SELEX (17) or positive-negative SELEX (18), gRNA variants can be created that can function on more than one DNA target site or that can distinguish between highly related DNA sequences to improve editing specificity and reduce off target editing concerns. The approach should also be amenable to optimizing a range of CRISPR-based editing systems. The availability of numerous functional gRNAs that work efficiently in mammalian cells will also allow for improved computation methods to predict which gRNA variant(s) will be optimal for particular research or medical applications as well as aid in our understanding why certain gRNAs work efficiently in vitro but not in vivo. The ability to modify the sequence of gRNA aptamer domains yet still create highly functional CRISPR-based editing agents will greatly facilitate the development of more efficient, higher resolution and more precise RNA and DNA editing.

### Materials & Methods:

Pools & Sequences:

All primers and templates were ordered through Integrated DNA Technologies (IDT).

The degenerate library for generating novel guide sequences was ordered as a partially randomized single-stranded DNA template based on the native gRNA scaffold. The library consisted of constant 5′ and 3′ regions for use as handles to re-amplify the pool. The 5′ region corresponded to the fixed target DNA sequence, and the 3′ constant region corresponded to the terminal 20 nucleotides of the wild type guide stem loop 3. The variable regions contained the native gRNA nucleotide at each position at a frequency of 58% and a 10.5% (58 library) of being any of the four nucleotides (FIG. 1a)

Templates for individual, selected variant guides were ordered as overlapping single stranded DNA oligonucleotides fragments. The forward fragment of each guide also contained the T7 promoter sequence (5′-TAATACGACTCACTATA-3′ (SEQ ID NO: 564)) to facilitate in vitro transcription.

DNA target substrate for in vitro cleavage assays (Substrate 1) was prepared by PCR of the GFP gene from plasmid gfap-EGFP donor (Addgene) and purified using the Qiagen PCR Cleanup Kit.

Target substrate for TdT functional screens and TdT A-tailing assays (Substrate 2) were ordered as hybridizing pairs. For A-tailing assays, the 5′ end of the forward strand was ordered with a Cy5 label or was radiolabeled in house as described below. Substrates were ordered with dideoxythymidine at the 3′ end to block TdT addition of nucleotides to the substrate ends.

Guide RNA Library and Variant Clone Generation.

The starting guide SELEX libraries were generated by annealing 1.5 nmol of the single-stranded template libraries to 1 nmole of the 5′ primer (5′-TAATACGACTCACTATAGGCGAGGGCGATGCCACCTA-3′ (SEQ ID NO: 565)) in 10 mM Tris-HCl, pH 8.0, with 10 mM MgCl2 at 95° C. for 5 minutes and then snap-cooling on ice. The annealed oligonucleotides were extended to full length with Exo(−) Klenow (NEB) according to the manufacturer's protocol, phenol-chloroform extracted, and subsequently concentrated and desalted with an Amicon-10 KDa Ultra-0.5 mL (Millipore) using 10 mM Tris pH 7.5 with 0.1 mM EDTA for washes. The DNA libraries were transcribed in vitro following manufacturer's protocol, using 250 pmol of DNA and 2 mM each NTP (NEB). Resulting RNA libraries were DNAse treated (NEB), phenol-chloroform extracted, concentrated, and desalted with an Amicon 10 KdA Ultra-0.5 mL and then purified using 12% denaturing polyacrylamide gel electrophoresis (PAGE). Excised RNA was eluted overnight in TE (10 mM Tris-Cl pH 8 with 1 mM EDTA) at 4° C. and desalted with an Amicon 10 kDa Ultra-0.5.

Overlapping oligonucleotides comprising each variant guide was PCR amplified using Phusion HF (NEB) following manufacturer's protocols and purified using a QIAquick PCR purification kit (Qiagen) (Wang et al. Nat Commun. 2020 Jan. 3; 11(1):91. doi: 10.1038/s41467-019-13765-3). PCR templates were transcribed with T7 polymerase for 2.5 hours at 37° C. Transcription reactions (50 uL) contained 2-4 μM template DNA, 200 units of T7 polymerase, 1 μg/mL pyrophosphatase (Roche), 5 mM NTPs, 30 mM Tris-Cl (pHRT 8.1), 25 mM MgCl2, 10 mM dithiothreitol (DTT), 2 mM spermidine, and 0.01% Triton X-100. Reactions were treated with DNase (Lucigen) for 30 minutes at 37° C., loaded onto a 12% denaturing urea-polyacrylamide gel, excised and eluted overnight in TE at 4° C. Triphosphates were removed with 10 units of calf intestinal phosphatase (NEB) as previously described (Sternberg et al. RNA. 2012 April; 18(4):661-72.)

Selection for Cas9 Binding

In vitro selection for binding was initially performed by isolation of bound RNA—protein complexes filtered through a 25 mm 0.45 μm nitrocellulose membrane (Schleicher & Schuell Biosciences). Rounds 1 and 2 were performed by incubating 1 nmole of each RNA library with 0.1 nmole of SpCas9 (NEB and ThermoFisher) in selection buffer (20 mM HEPES pH 7.4, 100 mM NaCl, 1 mM MgCl2, and 0.01% bovine serum albumin (BSA)) at 37° C. to generate ribonucleoprotein complexes (RNPs). RNPs were filtered through a nitrocellulose membrane, and the RNAs were extracted via phenol:chloroform:isoamyl alcohol (25:24:1) and ethanol precipitation in 0.3 M sodium acetate and 2.5× volumes 100% ethanol. 50% of the extracted RNA was revere transcribed (RT) with 100 pmol of the 3′ primer, 10 nmol dNTPs, and 20 units of AMV Reverse Transcriptase (Roche) according to the manufacturer's protocol. The RT reaction was PCR amplified with 500 pmol of 5′ and 3′ primers using standard PCR conditions. Reactions were then desalted and purified with a Qiagen PCR Purification Kit according to the manufacturer's protocol.

The resulting PCR product was utilized to generate the gRNA libraries necessary for subsequent rounds of selection, following the transcription conditions for Guide RNA Library Generation, above but using only 100 pmol selection round input.

Validating TdT-based Capture of Cleavage Capable RNP Complexes

To ensure the TdT-based scheme would work for isolating cleavage-capable variant guides, the inventors set up a radiolabeled A-tailing assay. The DNA target for these assays, Substrate 2, was synthesized as forward and reverse complementary oligonucleotides with dideoxythymidine at the 3′ ends to block nucleotide addition by TdT. 20 pmol Substrate 2 was end labeled using 20 U T5 Polynucleotide Kinase (NEB) and 20 Ci (5000 Ci/mmole) adenosine 5-[-32P]-triphosphate (GE Healthcare) at 37° C. for 1 hour. Radiolabeled DNAs were cleaned with Bio-Spin P30 columns as described above.

20 pmol of w.t. scaffold RNA or Round 0 RNA were incubated with 20 pmol of either active SpCas9 or an inactive “dead” variant (dCas9; NEB) in a reaction that contained NEB buffer 3.1 supplemented with 0.025% Tween and incubated at 37° C. for 1 hour to enable for RNP formation. Trace amounts of radiolabeled DNA was added to the RNP reaction mixture and incubated at 37° C. for 1. The reaction mix was then supplemented with a TdT mix that consisted of 5×TdT Buffer to a final concentration of 1×, 5 mM CoCl2, 1 mM dATP, and 100 U TdT and allowed to react at 37° C. for 30 minutes. The samples were cleaned through Amicon 30 kDa Ultra-0.5 columns to remove excess nucleotides, and 1 pmol biotinylated Oligo(dT) probe was added to each reaction. After incubation at 37° C. for 15 minutes, excess probe was removed through Amicon 30 kDa Ultra-0.5 columns, and the eluted complexes were added to 2 uL of Streptavidin T1 Dynabeads in NEB Buffer 3.1 supplemented with 0.005% Tween-20 (Sigma) and incubated at room temperature for 1 hour with rotation. Complexes bound to the magnetic beads were sequestered and washed 3× in NEB Buffer 3.1 supplemented with 0.005% Tween-20. Washes were collected and both bead fractions and wash fractions were mixed with Safety-Solve scintillation fluid (Research Products International) and radiation levels were detected using a Tri-Carb 4810 TR scintillation counter (Perkin Elmer).

Selection for Cleavage Capable Variant Guides

Rounds 3-5 of the functional selection were performed by isolating cleavage capable RNA-protein complexes. Substrate 2, which had been synthesized with dideoxythymidine at the 3′ ends to prevent nucleotide addition by TdT, was the target of these rounds. Substrate 2 dsDNA was generated by annealing 1.0 nmol of the forward and reverse oligonucleotides in 10 mM TE at 95° C. for 5 minutes and then snap-cooling on ice. Reactions were desalted and purified with a Qiagen PCR Cleanup Kit and resuspended in TE.

Potential RNPs were formed by incubating the in vitro transcribed gRNA libraries from binding selection Round 2 with SpCas9 at an equimolar ratio of 0.1 nmol at 37° C. for 30 minutes in NEB buffer 3.1. 3 pmol of 3′-end blocked Substrate 2 target DNA was then added to the RNP reaction mix for an additional 30 minute incubation. Following RNP-DNA cleavage complex formation, 100 U of recombinant E. coli TdT (Sigma), 5× Reaction buffer to a final concentration of 1×, 5 mM CoCl₂ and 1 mM dATP was added to the reaction and incubated at 37° C. for 30 minutes. Unincorporated nucleotides were removed with an Amicon 10 kDa Ultra-0.5, using 1×NEB Buffer 3.1 for washes. 1 pmol of a biotinylated Oligo(dT) (Promega) was added to the reaction mix and incubated at 37° C. for 15 minutes with rotation. Unbound probes were removed with an Amicon 30 kDa Ultra-0.5 using 1×NEB buffer 3.1 for washes. The biotinylated TdT-treated RNP-DNA complexes were mixed with 2 uL of Streptavidin T1 Dynabeads in NEB Buffer 3.1 supplemented with 0.005% Tween-20 (Sigma) and incubated at room temperature for 1 hour with rotation. Complexes bound to the magnetic beads were sequestered and washed 3× in NEB Buffer 3.1 supplemented with 0.005% Tween-20. Target DNA was then degraded with DNase I, and the protein bound gRNA library was prepped for subsequent rounds as described above.

TABLE 4
Conditions for gRNA selection. The gRNA SELEX scheme
winnowed variant diversity through standard nitrocellulose
(‘Nitro’) filter-based SELEX for RNP formation and binding
to SpCas9 (Rounds 1-2), selection for binding to the target
DNA (Round 3), and then a functional selection for cleavage
permissive gRNA variants (Rounds 4-5). Concentrations
of components and buffer conditions are as indicated.

| Round | | | Protein | RNA | | DNA | |
|---|---|---|---|---|---|---|---|
| # | Scheme | Target | pM | pM | | pM | Buffer |
| Round | Nitro | Cas9 | .1 pM | 1:00 | | N/A | 1 |
| 1 | | | | PM | | | |
| Round | Nitro | Cas9 | .1 pM | 1:00 | | N/A | 1 |
| 2 | | | | PM | | | |
| Round | DNA | DNA | .1 pM | 1:00 | | 001 pM | 2 |
| 3 | Binding | Target | | PM | | | |
| Round | TDT | Cas9 & | .1 pM | | uM | 001 pM | 2 |
| 4 | Cas9 | DNA | | | | | |
| Round | TDT | Cas9 & | .1 pM | .1 | uM | 001 pM | 2 |

| | | |
|---|---|---|
| 5 | Cas9 | DNA |

Sequencing and Analysis.

Round 2 of the binding selection and Round 5 of the TdT-based selection was PCR amplified with adapter primers and cleaned by Qiagen PCR Cleanup Kit. 500 ng of amplified DNA was submitted for Amplicon EZ sequencing (GeneWiz). The returned sequences were frequency ranked through FastAptamer (Donald Burke Laboratory (Khalid K. Alam, Jonathan L. Chang & Donald H. Burke “FASTAptamer: A Bioinformatic Toolkit for High-Throughput Sequence Analysis of Combinatorial Selections.” Molecular Therapy Nucleic Acids. 2015. 4, e230; DOI: 10.1038/mtna.2015.4)). Sequence alignments and phylogenetic trees were performed using Geneious (BioMatters Ltd.).

In Vitro Cleavage Assay

Selected variant gRNAs were transcribed and purified as described above. For cleavage assays, 5 pmol each variant gRNA was mixed with equimolar amounts of SpCas9 and incubated at room temperature for 15 minutes. 0.5 pmol DNA target Substrate 1 was added to each tube, and the reactions were incubated at 37° C. for 30 minutes. Cleavage reactions were treated with 1 uL of 20 mg/ml proteinase K at 37° C. for 30 minutes and then loaded onto 3% agarose gels stained with SYBR Safe.

Flow Cytometry Assays

Different rounds from the gRNA selection, as well as Round 0 and the w.t. scaffold were transcribed and purified as described above. For flow cytometry assays, 5 pmol each variant gRNA was mixed with equimolar amounts of SpCas9 and incubated at room temperature for 15 minutes. 0.5 pmol DNA target Substrate 2 labeled with Cy5 at the 5′ end was added to each tube, and the reactions were incubated at 37° C. for 30 minutes. Reactions were mixed with 1 uL Streptavidin T1 Dynabeads and incubated at room temperature for 30 minutes. The beads were washed with NEB Buffer 3.1 supplemented with 0.002% and analyzed on a CytoFlex flow cytometer (Beckman Coulter).

RNA/Protein Radiolabeled Nitrocellulose Binding Assay.

100 pmol of RNAs were treated with Calf Intestinal Phosphatase (CIP), of which 3 pmol were subsequently end labeled using 20 U T4 polynucleotide kinase (NEB) and 20 Ci of 5000 Ci/mmol adenosine 5-[-32P]-triphosphate (GE Healthcare). Radiolabeled RNAs were cleaned with Bio-Spin P30 columns (BioRad) and eluted in TE to remove unincorporated nucleotides. The dissociation constants were determined through a double-filter nitrocellulose binding assay. Assay methodology, fraction of bound RNA and non-specific background corrections were conducted and assessed as previously described (Wong and Lohman, “A double-filter method for nitrocellulose-filter binding: application to protein-nucleic acid interaction”. Proc Natl Acad Sci USA. 1993 Jun. 15; 90(12): 5428-5432).

### REFERENCES FOR EXAMPLE 2

1. M. Jinek et al., A programmable dual-RNA-guided DNA endonuclease in adaptive bacterial immunity. Science 337, 816-821 (2012).
2. P. Mali et al., RNA-guided human genome engineering via Cas9. Science 339, 823-826 (2013).
3. L. Cong et al., Multiplex genome engineering using CRISPR/Cas systems. Science 339, 819-823 (2013).
4. M. Jinek et al., RNA-programmed genome editing in human cells. Elife 2, e00471 (2013).
5. J. A. Doudna, The promise and challenge of therapeutic genome editing. Nature 578, 229-236 (2020).
6. B. A. Sullenger, RGEN Editing of RNA and DNA: The Long and Winding Road from Catalytic RNAs to CRISPR to the Clinic. Cell 181, 955-960 (2020).
7. S. B. Thyme, L. Akhmetova, T. G. Montague, E. Valen, A. F. Schier, Internal guide RNA interactions interfere with Cas9-mediated cleavage. Nat Commun 7, 11750 (2016).
8. T. Wang, J. J. Wei, D. M. Sabatini, E. S. Lander, Genetic screens in human cells using the CRISPR-Cas9 system. Science 343, 80-84 (2014).
9. S. Riesenberg, N. Helmbrecht, P. Kanis, T. Maricic, S. Paabo, Improved gRNA secondary structures allow editing of target sites resistant to CRISPR-Cas9 cleavage. Nat Commun 13, 489 (2022).
10. R. E. Martell, J. R. Nevins, B. A. Sullenger, Optimizing aptamer activity for gene therapy applications using expression cassette SELEX. Mol Ther 6, 30-34 (2002).
11. C. Tuerk, L. Gold, Systematic evolution of ligands by exponential enrichment: RNA ligands to bacteriophage T4 DNA polymerase. Science 249, 505-510 (1990).
12. A. D. Ellington, J. W. Szostak, In vitro selection of RNA molecules that bind specific ligands. Nature 346, 818-822 (1990).
13. J. M. Layzer, B. A. Sullenger, Simultaneous generation of aptamers to multiple gamma-carboxyglutamic acid proteins from a focused aptamer library using DeSELEX and convergent selection. Oligonucleotides 17, 1-11 (2007).
14. S. W. Lee, B. A. Sullenger, Isolation of a nuclease-resistant decoy RNA that can protect human acetylcholine receptors from myasthenic antibodies. Nat Biotechnol 15, 41-45 (1997).
15. A. W. Kahsai et al., Conformationally selective RNA aptamers allosterically modulate the beta2-adrenoceptor. Nat Chem Biol 12, 709-716 (2016).
16. C. D. Richardson, G. J. Ray, M. A. DeWitt, G. L. Curie, J. E. Corn, Enhancing homology-directed genome editing by catalytically active and inactive CRISPR-Cas9 using asymmetric donor DNA. Nat Biotechnol 34, 339-344 (2016).
17. R. White et al., Generation of species cross-reactive aptamers using “toggle” SELEX. Mol Ther 4, 567-573 (2001).
18. J. Ishizaki, J. R. Nevins, B. A. Sullenger, Inhibition of cell proliferation by an RNA ligand that selectively blocks E2F function. Nat Med 2, 1386-1389 (1996).

TABLE 2
Exemplary S. pyogenes gRNA sequences.

| Variant | Cleavage | | SEQ ID |
|---|---|---|---|
| ID | Capable | Sequence | NO: |
| w.t. | Yes | GUUUUAGAGCUAGAAAUAGCAAGUUAAAAUAAGGCUA | 584 |
| gRNA | | GUCCGUUAUCAACUUGAAAAAGUGGCACCGAGUCGGUG | |
| | | C | |
| 1 | Yes | GUUUUAGAGCUAGAAAUAGCAUGUUAAAAUCAGACUA | 345 |
| | | GUUCGUUACCAAUUUGCAGAAGUGGCACCGAGUCGGUG | |
| | | C | |
| 2 | Yes | GUUUUACAGCUAGAGAUAGCAAGUUAAAAUAAGGCUA | 346 |
| | | GUUCGUUACCAACGAGAACACGUGGCACCGAGUCGGUG | |
| | | C | |
| 3 | | GGUUAACCGCUCUAAACAGCGAGUGAAUUCGAGGCUUG | 347 |
| | | UGCACAAUCACCCGUUAUCGGUGGCACCGAGUCGGUGC | |
| 4 | Yes | GGUUUAGAGGUAGAAAUACCAAGUUAAAGUAAGGCUA | 348 |
| | | GACCGUUAUUAUCGUGAAUGCGUGGCACCGAGUCGGUG | |
| | | C | |
| 5 | | GCUCGAUAGCUAUUUAGAUAAAGUUAAAUUAAGGCUAC | 349 |
| | | GCGGGUAACAACUCGACCCCGUGGCACCGAGUCGGUGC | |
| 6 | Yes | GUUUUAUAGCCAGAAAUGGCGAGUUAAAAUAGGGCCAG | 350 |
| | | UCCGAUAUCAACUUAAUCCGUGGCACCGAGUCGGUGC | |
| 7 | Yes | GUCUUAGAGCUAGACCUAGCAAGUUAAAAUAAGGCGAG | 351 |
| | | UUCGUUAUCAACCAUUUCGAGUGGCACCGAGUCGGUGC | |
| 8 | Yes | GUUUUCGAGCCAGAAAUGGCAAGUGAAAAUAAGGCAAG | 352 |
| | | UCCGUUAGCGACUGUUCACAGUGGCACCGAGUCGGUGC | |
| 9 | | GCUUAAGUGCAAGAAAGUGCAAAUUAGGACAAGGCUAU | 353 |
| | | CAAGUCCUCAACCUCAACUGGUGGCACCGAGUCGGUGC | |
| 10 | Yes | AUUUUAGGAGUUAGAAAUAACAAGUCUAAAUAAGUCU | 354 |
| | | AGUACGCUAUCAACUGGAACAUGUGGCACCGAGUCGGU | |
| | | GC | |
| 11 | Yes | GUUUAAGAGCCAUAACAAGUAAGUUUAAAUAUGGCAU | 355 |
| | | GUCCGUUAUCAACAUCACACUGUGGCACCGAGUCGGUG | |
| | | C | |
| 12 | | UCUUGGCAGCCGACAGCAGUAAGUUAACUUAUGUUUAG | 356 |
| | | UCUGACCUUACCCUUUACCGGUGGCACCGAGUCGGUGC | |
| 13 | | GCUUUCGAGCCAGUGACGGUAAGUGAAAUCGAGGCUAG | 357 |
| | | UCCGUUAGCCCAUUGAACUGGUGGCACCGAGUCGGUGC | |
| 14 | | GUGGUCGGACUAUACAUAGCUAGUUCCGAUAAGUCUAG | 358 |
| | | AUCGCAGGCAAACUGCUCCGGUGGCACCGAGUCGGUGC | |
| 15 | Yes | GUUUUGGAGCUAGUUUGAGCAAGUCAAAAUAAGGCGA | 359 |
| | | GUCCGUUAUUAACUUGAACAUGUGGCACCGAGUCGGUG | |
| | | C | |
| 16 | Yes | GUUUUAGAGCUAGAAAUAGCAAGUUAAAAUAAGACUA | 360 |
| | | GUCCGUGAGUAACUUGAAGAUUGGGCACCGAGUCGGUG | |
| | | C | |
| 17 | | GUCAACUAGCUAGAACUAACAAGUGAAGAGAAUUCGAG | 361 |
| | | UUAGUUUUCACCGCUAUCCCGUGGCACCGAGUCGGUGC | |
| 18 | Yes | GUUUUAGAGCGUACAUGCGCAAGUUAAAAUAAGGCAAU | 362 |
| | | UCCGUUAACAACUUAACACAGUGGCACCGAGUCGGUGC | |
| 19 | Yes | GUUUUCAAGCUAAAAAUAGCAAGUGAAAAUAAUGCUA | 363 |
| | | GUCAGUAGGCAACUUCCAGCAGUGGCACCGAGUCGGUG | |
| | | C | |
| 20 | | GCGAUCGAGCUAGACAUCGCAAGUUCGCAAAAUACGAG | 364 |
| | | UGCACCAGGCACUUCAGAGGGUGGCACCGAGUCGGUGC | |
| 21 | Yes | GUUUUAGAGUUAGGAAACACAAGUUAAAAUAGGGCUA | 365 |
| | | GUCCGGAAACCGUUAGAACACGUGGCACCGAGUCGGUG | |
| | | C | |
| 22 | Yes | GUUUUAGAGAUCGGAAGAUCAAGUUAAAAUAAGGCUA | 366 |
| | | GUCCCGUUACAACGUGGAACCGUGGCACCGAGUCGGUG | |
| | | C | |
| 23 | Yes | GCUAUAGAGCUAGAAAUAGCAAGUUAUAAUAAGGCAA | 367 |
| | | GACCGUUAUCAAACCGAAAUGUUGGCACCGAGUCGGUG | |
| | | C | |
| 24 | | GUGUUCGAGCUAGGCUUAGCAAGUGAACAUUAGGCGAG | 368 |
| | | UCCGUUAUCAACUUGGAACAGUGGCACCGAGUCGGUGC | |
| 25 | Yes | GUCUUAGAGCUAAUUUUAGCAAGUUAAAAUCAGGCUAG | 369 |
| | | UCCGUUAUCAACUUGAUCAAGUGGCACCGAGUCGGUGC | |
| 26 | | GCCAUAGAGCUAGACAUACCAAGGCAAAACAUUGCCAG | 370 |
| | | UGCGCUACCAACCAAAAGCGGUGGCACCGAGUCGGUGC | |
| 27 | | AUUCUGGUGACAUUAAACGCCAGUUCGCGUUAUGCAAU | 371 |
| | | CCCGUUCCAUACUUGAACCGGUGGCACCGAGUCGGUGC | |
| 28 | Yes | GUUUUAGAGCUAACAAAAGCAAGUUAAAAUAAGGCUA | 372 |
| | | GACCGUUUAUCAACCUUUAAUGGUGGCACCGAGUCGGU | |
| | | GC | |
| 29 | | GUUAUAGUGUUAGGAAUGGCCCCCUAGCAUUACGUGUC | 373 |
| | | UCCGCCAUUAAUUACUACACGGGGCACCGAGUCGGUGC | |
| 30 | | UGUGAGGAUUCAAAAACAUCCAUGUACAUUAAGCCUAG | 374 |
| | | UCAGUUACCAAACCCCUGCGGUGGCACCGAGUCGGUGC | |
| 31 | Yes | GUUUUAGAGUUCAUAAUAACAAGUUAAAAUAAGGCUA | 375 |
| | | GACCGUGAUCAUCCGGACACUGUGGCACCGAGUCGGUG | |
| | | C | |
| 32 | Yes | CUUUGAGAGCUAGAAAUAGCCGGUUCAAAUAAGGCGCG | 376 |
| | | UCCGUUAACAACCUGUCACUGGUGGCACCGAGUCGGUG | |
| | | C | |
| 33 | | GUGUUUGAGCGCGAAAAAGCCAGACAUAAAUCGGCAAG | 377 |
| | | UCGAAUUUCAACCCGUAGCGGUGGCACCGAGUCGGUGC | |
| 34 | | CACUCACAGCUAGAAAUAGCAAGUUGAAUUAAGGGUAG | 378 |
| | | UACGUGAGCAACCUUCAUUGGUGGCACCGAGUCGGUGC | |
| 35 | | GAGAUAGUGCUCCAAAUGGGAGAUCACAAUCAAGUUAG | 379 |
| | | UCGUUUAUCAACCUGCAUGUGUGGCACCGAGUCGGUGC | |
| 36 | | UUGUUCGAGCUAGCAUUAGCAAGUGAAACUAGCGAUUA | 380 |
| | | ACCCUUCUUUCCUCCCACUGGUGGCACCGAGUCGGUGC | |
| 37 | | UGGCUGUAGCAGGAAAUGGCUAAUGCAAGUUAGGGUA | 381 |
| | | GGCCGCGAGCAACUCGAACCGCGGGCACCGAGUCGGUG | |
| | | C | |
| 38 | Yes | GUUUUAGAGGCCACAAUACCGAGUUAAAAUAAGGCUUG | 382 |
| | | UCCGUUAUCAACUUUGCAACGUGGCACCGAGUCGGUGC | |
| 39 | Yes | GUUUUAGGGUUCAAAAUAACAAGUUAAAAUAAGGCUU | 383 |
| | | GUCCGUUAGCAACUUGAAUACGUGGCACCGAGUCGGUG | |
| | | C | |
| 40 | Yes | AUUUUACCGCUCGCAAGAGCAAGUUAAAAUAAGGCUCU | 384 |
| | | CCGAUAUCAACUUGUAACAGUGGCACCGAGUCGGUGC | |
| 41 | | GACUCAACGCUAGAAAUAGCAAAGUCAAAUUUGGCAAG | 385 |
| | | GCAGUCAUGAACCCUAUACGGUGGCACCGAGUCGGUGC | |
| 42 | Yes | GUUUUCGAGCUAGAAAUAGAUAGUGAAAAUAAGCCUU | 386 |
| | | GUGCGUCACCAACAUGAAACAGUGGCACCGAGUCGGUG | |
| | | C | |
| 43 | | AUCUCUGCGCCAGAAUUCGCCAGUGAAAAUAAGCCUAG | 387 |
| | | GUCAGCGCCGAACUAAGACGUGGGCACCGAGUCGGUGC | |
| 44 | Yes | CUUUUAGAGUUAGUAAUAAUCAGUUAAAAUAAGGCAA | 388 |
| | | GUCCGUGAUCAACCGGAAGGGUGUGUCACCGAGUCGGU | |
| | | GC | |
| 45 | | GUUCUAGAGCACGAAAGAGCCUGUUAGAACAGUACACG | 389 |
| | | GCCGUCAUCAACCUUACACGGUGGCACCGAGUCGGUGC | |
| 46 | | CGUACCUACGUAGAAACGGCUAGGAAAAAUUGCGCUAG | 390 |
| | | UGGUGUAUCACCUAUAACAGGGGGCACCGAGUCGGUGC | |
| 47 | Yes | GUUUUAGGGCUAUAAAUAGCGAGUUAGAAUAAGGCUA | 391 |
| | | GUCCGUGAGCAACUUGGCAAGUGUGGCACCGAGUCGGU | |
| | | GC | |
| 48 | | GUUUGAGAGCUACAAGUAGCCAGUUCAAACAUAAAUUG | 392 |
| | | UCCCAUAUCAACUUGAGUCAGUGGCACCGAGUCGGUGC | |
| 49 | | AGCUUGGAGCUAGAAUUAGCAAGCUAAGUUCAAAGACC | 393 |
| | | UUCGGAAAACCCUUCAUUCGGUGGCACCGAGUCGGUGC | |
| 50 | | GUUUUAAAGCUAGCCAAGAACACUAAUUGGCAGGUUAG | 394 |
| | | UACGUGCUCAUCUUGAUGCGUGGGCACCGAGUCGGUGC | |
| 51 | | GGUAUAGAGCUAGAAAUAGCCGCCCAGAAUCAGCCCAG | 395 |
| | | UGCGUUAUUAACCGUUUGCGUGGGCACCGAGUCGGUGC | |
| 52 | | GUUGGCGAUUCACGUAAAGCGCAUUCAGUUAACGUCUG | 396 |
| | | UGGGGUGUCCACUUUAUCACGUGGCACCGAGUCGGUGC | |
| 53 | | GUUUUUGAGCAAGACUUUGUUAGGCAGCCUAAGGGUGG | 397 |
| | | UCCGUUACGACCCUGUCCCGGGGGCACCGAGUCGGUGC | |
| 54 | | GGUGCUGAGGCAGCACUCGAAAGCUUCAGCAAGUCUAG | 398 |
| | | UCGGUAGUGGACCGGAUACCGUGGCACCGAGUCGGUGC | |
| 55 | | GUUCCCGAGGUAGCAGUAGCACUUCAAAAUAAGUAGUU | 399 |
| | | AACGUUAGCCACGCUAUGCGGUGGCACCGAGUCGGUGC | |
| 56 | | GUAUUAGACCUUUCCGUUGGAAACUAGGCGAAAGUUCG | 400 |
| | | CCCGUUAUAAGCUUGCAGUGGUGGCACCGAGUCGGUGC | |
| 57 | | GUUUUAGACAAAUAGAUCAAACGUCGUCCUAAGUCGAG | 401 |
| | | UGCAUCCUAAACACACAAAUGGGGCACCGAGUCGGUGC | |
| 58 | | GUUUCCGCGGAAGAGAUUGCAAGGAUAAGUUAGUGUG | 402 |
| | | GCCCAUUCAAAACCUAGCAGGUGGGCACCGAGUCGGUG | |
| | | C | |
| 59 | | GUUUUCAAUCUAGAAGCGACAUCAUACCCUAAGUCUGG | 403 |
| | | UCCUUCAUAAACUUGCCCCUGCGGCACCGAGUCGGUGC | |
| 60 | | GGUUGGGUACUUGUAAUACCACCCCCAAUCUAAGUUAG | 404 |
| | | ACCGCAAGAACCUAUAAUCGGUGGCACCGAGUCGGUGC | |
| 201 | Yes | GUUUUAGAGCAAGAAAUUGCAAGUUAAAAUAAGGCUA | 405 |
| | | GACCGUUAUCAACGUGACUGUGUGGCACCGAGUCGGUG | |
| | | C | |
| 202 | Yes | GUUUUAUAGCUAGCAAUAGCAAGUUAAAAUAAGGCUA | 406 |
| | | GUCCGUUAUGAACGUGAAACCGUGGCACCGAGUCGGUG | |
| | | C | |
| 203 | Yes | AUUUUAGGAGUUAGAAAUAACAAGUCUAAAUAAGUCU | 407 |
| | | AGUACGCUAUCAACUGGAACAUGUGGCACCGAGUCGGU | |
| | | GC | |
| 204 | | GGUAUAGAGCUAGAAAUAGCCGCCCAGAAUCAGCCCAG | 408 |
| | | UGCGUUAUUAACCGUUUGCGUGGGCACCGAGUCGGUGC | |
| 205 | Yes | GUUUUAGAGCUAGAAGUAGCAAGUUAAAAUAAGGCUA | 409 |
| | | GACCGUCAUCAACCUUCAUGCGUGGCACCGAGUCGGUG | |
| | | C | |
| 206 | Yes | GUUUUAUUGCUAGAAAUAGCAAGUUAAAAUAAGUCUA | 410 |
| | | GUGCGUUAACAACGUGCCCACGUGGCACCGAGUCGGUG | |
| | | C | |
| 207 | Yes | GUUUUAGUGCGAGAAACCGCAAGUUAAAAUAAGACUAG | 411 |
| | | UCCGUUUGCAACUGUGACAUGUGGCACCGAGUCGGUGC | |
| 208 | Yes | GUUUUGCAGCUAAAAUUAGCAUGUCAAAAUAAGGUUCC | 412 |
| | | UCCGGUGACAACGUGAAUACGUGGCACCGAGUCGGUGC | |
| 209 | Yes | UUUUUAGAACUAGAAAUAGCAAGUUAAAAUAAGGCAA | 413 |
| | | GUCCAUGAUCAACGGUGACCGUGUGGCACCGAGUCGGU | |
| | | GC | |
| 210 | Yes | GUUUUGCAGCGAGAAAUCGCAGGUCAAAAUAAGUCUGG | 414 |
| | | UACGCAAUCAACGUGAAAACGUGGCACCGAGUCGGUGC | |
| 211 | | GUUUUCAAUCUAGAAGCGACAUCAUACCCUAAGUCUGG | 415 |
| | | UCCUUCAUAAACUUGCCCCUGCGGCACCGAGUCGGUGC | |
| 212 | Yes | GUUUUAGAGCUAGAAAUAGCAAGUUAAAAUAAGGUUA | 416 |
| | | AUUCGUUAACCAACGAGAAACGCGUGGCACCGAGUCGG | |
| | | UGC | |
| 213 | Yes | GUUUUCAAGCUAAAAAUAGCAAGUGAAAAUAAUGCUA | 417 |
| | | GUCAGUAGGCAACUUCCAGCAGUGGCACCGAGUCGGUG | |
| | | C | |
| 214 | Yes | GUUUUAUACCUAGAAAUAGGAAGUUAAAAUAAGUCUA | 418 |
| | | GUCCGUUACCAACGUGAAUCCGUGGCACCGAGUCGGUG | |
| | | C | |
| 215 | Yes | GUUUUACAGCCAGAAAUGGCAAGUUAAAAUAAGGCCAG | 419 |
| | | UCCGUUAACACUUUUCACCAGUGGCACCGAGUCGGUGC | |
| 216 | Yes | GUUUUCCAGCUAGCAAUAGCAAGUGAAAAUAAAGCUAG | 420 |
| | | UCCGUUCUCACCUUGACACGGGGGCACCGAGUCGGUGC | |
| 217 | | GCUUAAGUGCAAGAAAGUGCAAAUUAGGACAAGGCUAU | 421 |
| | | CAAGUCCUCAACCUCAACUGGUGGCACCGAGUCGGUGC | |
| 218 | | GUGGUCGGACUAUACAUAGCUAGUUCCGAUAAGUCUAG | 422 |
| | | AUCGCAGGCAAACUGCUCCGGUGGCACCGAGUCGGUGC | |
| 219 | Yes | GUUUUAUAGCCAGAAAUGGCGAGUUAAAAUAGGGCCAG | 423 |
| | | UCCGAUAUCAACUUAAUCCGUGGCACCGAGUCGGUGC | |
| 220 | | AUUGUAGUUGCCAGAAACAACAAGUCAAGAUAGCGAGU | 424 |
| | | CCGUCCUCACCCGGUGACCCCGGCACCGAGUCGGUGC | |
| 221 | Yes | GUUUCAGUGCUAGAAUUAGCAAGUUGAAAUAAGGUUA | 425 |
| | | UUCCGUGCCUGCCUGGACAGGGUGGCACCGAGUCGGUG | |
| | | C | |
| 222 | | CGUAUGCAACUAACUAUAGCAUACUAGAGAUUGGCAAG | 426 |
| | | UCGCUACUUACCUAGGUGCCGUGGCACCGAGUCGGUGC | |
| 223 | Yes | AUUUUACCGCUGGAAACAGCAAGUUAAAAUAACGCUAG | 427 |
| | | ACGGUGAUCAGCGUGCAAACGUGGCACCGAGUCGGUGC | |
| 224 | Yes | GCUUUAGCGCUAGAAAUAGCAAGUUAAAGUAAAGCGAG | 428 |
| | | UCUGUGAUCAACGCGAAAACGUGGCACCGAGUCGGUGC | |
| 225 | Yes | GUUUUAGAGCUAGAAGUAGCAAGUUAAAAUAUGGCUA | 429 |
| | | GUCCGUGAGCAACCCGAAGUGGUGGCACCGAGUCGGUG | |
| | | C | |
| 226 | Yes | GUUUUGGACCUAGAAAUAGGACGUCAAAAUAAGCCUAG | 430 |
| | | UGCGUGCUCAACCUGAAAUGGUGGCACCGAGUCGGUGC | |
| 227 | Yes | GUUUUCAUGCUAGGACUAGCAAGUGAAAAUAAGUCUCG | 431 |
| | | UACGUUGUCAACCUGAUCGGGUGGCACCGAGUCGGUGC | |
| 228 | | UUUUUCGAUGUAGAACUACAAAGUGAAAAGAGGUCUA | 432 |
| | | GUCACCUAUCCCCCUGUGCCGGCGGCACCGAGUCGGUG | |
| | | C | |
| 229 | | GCUCGAUAGCUAUUUAGAUAAAGUUAAAUUAAGGCUAC | 433 |
| | | GCGGGUAACAACUCGACCCCGUGGCACCGAGUCGGUGC | |
| 230 | Yes | UUUUUAGAGCCAGAAAGAGCAAGUUAAAAUAAGGCAA | 434 |
| | | GUCCGUUUUCAACGUAACCACGUGGCACCGAGUCGGUG | |
| | | C | |
| 231 | | UGUAUGCAACUAACUAUAGCAUACUAGAGAUUGGCAAG | 435 |
| | | UCGCUACUUACCUAGGUGCCGUGGCACCGAGUCGGUGC | |
| 232 | Yes | GUUUUAGCGCCAAAAAAAGCAAGUUAAAAUAAGGCGAG | 436 |
| | | UCCGCUAUCAACCUGAAACGGUGGCACCGAGUCGGUGC | |
| 233 | | GCGCUAAAGUUAGACUUAGCAAGUUAGGUUCCGUCUGU | 437 |
| | | UCCCGAGUCAACUUGGUGCAGUGGCACCGAGUCGGUGC | |
| 234 | Yes | GUUUUAGAGCCAGCAAUGGCAAGUUAAAAUAGGGCUUG | 438 |
| | | UCCGUGAUCAACUUGAACAAGGGGCACCGAGUCGGUGC | |
| 235 | Yes | GUUUUCGAUCAAGAAAUUGCAAGUGAAAACAAGGCAAU | 439 |
| | | CCCGUACCCAACCUGAAACGGUGGCACCGAGUCGGUGC | |
| 236 | | AGUUACUAAUCGCUCGUGUAACUUAGCGUAACCUUCCG | 440 |
| | | CUCACCUGGUGCCGUGGCACCGAGUCGGUGC | |
| 237 | | GAGUUGAAACAACGAAUAGCAUAUUUCAAUAUGUUUA | 441 |
| | | UUCCGGUGCAACGUUGUACACGUGGCACCGAGUCGGUG | |
| | | C | |
| 238 | | GUCUUAGAGCUAGAAAUAGCAAGUUAAGAUAAGGCUA | 442 |
| | | GUCCAUCGUCCACCGCAGCCGGUGGCACCGAGUCGGUG | |
| | | C | |
| 239 | Yes | GUUUUAGAGCUAGAAAUAGCAAGUUAAAAUACGACUA | 443 |
| | | GUUCAUUAAUAGCAUGAAAACGUGGCACCGAGUCGGUG | |
| | | C | |
| 240 | Yes | GUUUUCGAGCCAGAAAUGGCAAGUGAAAAUAAGGCAAG | 444 |
| | | UCCGUUAGCGACUGUUCACAGUGGCACCGAGUCGGUGC | |
| 241 | | GUCAACUAGCUAGAACUAACAAGUGAAGAGAAUUCGAG | 445 |
| | | UUAGUUUUCACCGCUAUCCCGUGGCACCGAGUCGGUGC | |
| 242 | | GUUUUAGAGCUAAAAAUAGCAAGUUAAAAUAUGGGUG | 446 |
| | | CUCCCAUGUCGUCUCGACUGCGGGGCACCGAGUCGGUG | |
| | | C | |
| 243 | Yes | GUUUGAGAAAGUGAACCUUCAAGUUCAAAUAAGGUUU | 447 |
| | | GUCCGGUAUCAACUGGAAACAGUGGCACCGAGUCGGUG | |
| | | C | |
| 244 | | GUGUUUGAAAUCGCAAUAGCACGAUCACCUAAUGCUAG | 448 |
| | | UCCGCGACAAACGUGGUGCUGCCGCACCGAGUCGGUGC | |
| 246 | | GUUCUAGAUCUAUCAACAGCAAGUUGAAAGAAAGUUAG | 449 |
| | | AACGAUGGGAACUUUCACCCUCGGCACCGAGUCGGUGC | |
| 247 | | GUUUUAGAGCUUUCAGUAGCAAGUUAAAACAUGGCUAG | 450 |
| | | UCCGUUGCCAUCUGGUGCGCGUGGCACCGAGUCGGUGC | |
| 248 | | GGUUUAGACUUAGAUACGUUAAGUUAUAAACCCCCCAG | 451 |
| | | UGCGGUGUGAAGUGGAACGCCUGGGCACCGAGUCGGUG | |
| | | C | |
| 249 | Yes | AUUUUUGCGCUAGUAAUAGCAAGUAAAAAUAAGACUG | 452 |
| | | GUCCGUUACCAACCUGGAAGGGUGGCACCGAGUCGGUG | |
| | | C | |
| 250 | | GCGUGAGCACUCGAACUUGCAAGUAUCAACAAGGUGAG | 453 |
| | | UCCCCUGCCAUCGUGAAACGGUGGCACCGAGUCGGUGC | |
| 251 | Yes | GUUUUGGAGCUAGUUUGAGCAAGUCAAAAUAAGGCGA | 454 |
| | | GUCCGUUAUUAACUUGAACAUGUGGCACCGAGUCGGUG | |
| | | C | |
| 252 | | GUUUUAGAGCGGAAAUCGCAAGUUAAAAUAAGGCUGG | 455 |
| | | UCAAUCCUCAAGGUGCUCGCGUGGCACCGAGUCGGUGC | |
| 253 | | AACGUGGUGCUAGAUAUAAACUGUAAAUAGAAAGUUG | 456 |
| | | GUCUUUGGUGACGCUGUUCUCGCGGCACCGAGUCGGUG | |
| | | C | |
| 254 | Yes | GCUUUAGAGCUAAAAAUUAGCAAGUUAAAGUCAGGCUA | 457 |
| | | GUCCGUGCGGAACGUGCCCCUGUGGCACCGAGUCGGUG | |
| | | C | |
| 255 | | GUGGUCGGACUAUACAUAGCUAGUUCCGAUAAGUCUAG | 458 |
| | | AUCGCAGGCAACUGCUCCGGUGGCACCGAGUCGGUGC | |
| 256 | | UUGUUCGAUCAUGAAACAGCAAGGUAAAACAUCGCAAG | 459 |
| | | UUCGAUAACGGUUACGGUGCGUGGCACCGAGUCGGUGC | |
| 257 | | ACCUUAGAUGCUCGUAGUUGAAACUUCGAGUAGGACGG | 460 |
| | | UGCCCUUGUCACCUUGAGUGGUGGGCACCGAGUCGGUG | |
| | | C | |
| 258 | | CUUGUAGAGAUACGAAUAGCAGUGUAAGUUUGCGCUAG | 461 |
| | | UCAACUCGCAAUUGGUGCCGUGGCACCGAGUCGGUGC | |
| 259 | Yes | GUUUUAGAGCUAGCAAUAGCAAGUUAGAAUAAGGCGA | 462 |
| | | GACCGUUAUCAGCUGGAACCAGUGGCACCGAGUCGGUG | |
| | | C | |
| 301 | Yes | GUUUUAGAGCGCGAAAUCGCAAGUUAAAAUAAGACUAG | 463 |
| | | UGCGUUCACAACUUCAGCAAGUGGCACCGAGUCGGUGC | |
| 302 | Yes | GUUUUAGUGCUAAACUUAGCAAGUUAAAAUAAGGCAA | 464 |
| | | GUCCGUUAUAAACGAGAACCGGUGGCACCGAGUCGGUG | |
| | | C | |
| 303 | Yes | GUUUGAGUGCUAGUAAUAGCAAGUUCAAAUAAGGAUA | 465 |
| | | GACCGCAAACACCGUGAACAGGUGGCACCGAGUCGGUG | |
| | | C | |
| 304 | Yes | GUUUUCGCGCCAGAAACGGCAAGUGAAAAUAAGACUAG | 466 |
| | | UUCGUAAACCACUGGAAACGGUGGCACCGAGUCGGUGC | |
| 305 | Yes | GGUUUAGCGCUGUGAACAGCAAUUGAAACUAAACUUAG | 467 |
| | | UCGGUGACCAACUUGAACGUGGGGCACCGAGUCGGUGC | |
| 306 | Yes | GUUUGAGAACUAGAAAUAGAAAGUUCAAAUAAGGUUA | 468 |
| | | AUCCGUUAUCAACUUGAAACAGUGGCACCGAGUCGGUG | |
| | | C | |
| 307 | Yes | GUUUUAUCGGUAGAAAAACCAUGUUAAAAUAUGGCUA | 469 |
| | | GUCCGGUGACAACGGGAUGCCGUGGCACCGAGUCGGUG | |
| | | C | |
| 308 | Yes | GUUUUAGUGCUCGAAAGAGAAAGUUAAAAUAAGAACA | 470 |
| | | UUUCGCGAUCACCGUUAAUACGUGGCACCGAGUCGGUG | |
| | | C | |
| 309 | Yes | GUUUCACAGCGCGAAAUCGCAAGUUGAAAUAAGACUAG | 471 |
| | | UUCGGUAGCAACAUGACAAUGUGGCACCGAGUCGGUGC | |
| 310 | | AUUUUAGUGCUAGAAUUAGCAAGUUAAAAUUCGGUGA | 472 |
| | | CACCCUGCUCAUCUUGCAGGCGGGGCACCGAGUCGGUG | |
| | | C | |
| 311 | | GUUUUAGUGCUAGAAAUAGCAAGUUAAAAUUGGUCCA | 473 |
| | | GUCCAUGUGCCACGUGAACAUGUGGCACCGAGUCGGUG | |
| | | C | |
| 312 | Yes | AUUUUCUAGCUAGCAAUAGCAUGUGAAAAUAAGGCUAG | 474 |
| | | ACCGAUGUCAACUUGUUCGGGUGGCACCGAGUCGGUGC | |
| 313 | | GUGUUACCACUAGACUUAACAAGUGAAAGUAAUUCGAG | 475 |
| | | UUUGUUACCGGUCCGUAACGGUGGCACCGAGUCGGUGC | |
| 314 | Yes | GUUUUAGAGCGGGAAAACGCAUGUUAAAACAAGACUAG | 476 |
| | | UCCGUUACCACCGUUAAACCGUGGCACCGAGUCGGUGC | |
| 315 | Yes | GUUUUAGCGCUUGAAAAAGCAAGUUAAAAUAAGGCUA | 477 |
| | | GUCCGUUAGUUAACGGAACAUGUGGCACCGAGUCGGUG | |
| | | C | |
| 316 | Yes | AGUUUACAUUUUGGAAUAACAAGUUCAAAUAGGUCUA | 478 |
| | | AACCGUGCACAACUUGCAAGUUGGGCACCGAGUCGGUG | |
| | | C | |
| 317 | Yes | GUUUUAGUGCGAGAAUUCGCAAGUUAAAAUCAGUCAAA | 479 |
| | | UACGUUGUCACCGUGCAAUCGUGGCACCGAGUCGGUGC | |
| 318 | | GUGUUCGAGCUAGGCUUAGCAAGUGAACAUUAGGCGAG | 480 |
| | | UCCGUUAUCAACUUGGAACAGUGGCACCGAGUCGGUGC | |
| 319 | Yes | GUUUUAGUGCUAGAAAUGGCAAGUUAAAAUAAGACCA | 481 |
| | | GUUCGUUAUCUACCUGAGUGCGUGGCACCGAGUCGGUG | |
| | | C | |
| 320 | Yes | GUUUUAGAGAUAGAAAUAUCAAGUUAAAAUAACGUCA | 482 |
| | | GUCCGGUGUCAGCGACAAAGCGUGGCACCGAGUCGGUG | |
| | | C | |
| 321 | Yes | GUUUUCGCAGUAGCAAUACCAAGUGAAAAUAAGAUUAG | 483 |
| | | UCCGAAAUCAACGUGAAACCGUGGCACCGAGUCGGUGC | |
| 322 | | GCUUUACCGCGAGAGAUAGCAAGUUAAAAUACGCUACG | 484 |
| | | UACGGUUGCUAUGUGACAACGUGGCACCGAGUCGGUGC | |
| 323 | Yes | GUAUUCGAGUCAGAAAUGGCACGUGAAUAUAAGACUAG | 485 |
| | | UUCGUACUCAACUGGCAAGCGUGGCACCGAGUCGGUGC | |
| 324 | Yes | GUUUUAGAGCUAGAAAUAGCAAGUUAAAAUAAGACGA | 486 |
| | | GAUCGAUACCAACUUGAGAAUGUGGCACCGAGUCGGUG | |
| | | C | |
| 325 | Yes | GUUUUAGCGCAGAAAACUGUAAGUUAAAAUAAGGCUA | 487 |
| | | GAUCGUUAACAACUGGAAUCAGUGGCACCGAGUCGGUG | |
| | | C | |
| 326 | Yes | GUUUUAGAGCUAGCAAUCGCAAGUUAAAAUAAGGAUCG | 488 |
| | | UCCGUUAUCAACUUGAAAGAGUGGCACCGAGUCGGUGC | |
| 327 | Yes | GAUUUAGAGCUGGAAACAGCAAGUUAAAAUAAGGCUU | 489 |
| | | GUCCGUCAACAACUUGAAAACGUGGCACCGAGUCGGUG | |
| | | C | |
| 328 | | GUUGUAGAGCUAUAAAUAGCAAGUUACAAUAAGGCUA | 490 |
| | | GUCCGUACUAAGCGUUCAUAUGUGGCACCGAGUCGGUG | |
| | | C | |
| 329 | Yes | GUUUUAGAGCUAGAAAUAGCAAGUUAAAAUCAGGCUA | 491 |
| | | GUCCAAGAACAACAUCAACACGUGGCACCGAGUCGGUG | |
| | | C | |
| 330 | Yes | AUCUUAGAGCUAGAAAUAGCAAGUUAACAUAAGGCGAG | 492 |
| | | UCCGUUUACAACUUCCAUACGUGGCACCGAGUCGGUGC | |
| 331 | | UUUUUAGCGCUAGAACUAGCUCGUGUAAAAAUUCCUAG | 493 |
| | | UACGUUAUCAACUUAAUCGAGUGGCACCGAGUCGGUGC | |
| 332 | | GUAUUCGAGCUAGAAAUAGCAAGUGAAUACAAGGCUAA | 494 |
| | | UCCGUUAUCAACACGCCCCGGUGGCACCGAGUCGGUGC | |
| 333 | Yes | GUGUUAGAGCUAGAAAUAGCAAGUUAACGUAAGGCUA | 495 |
| | | GUCCGCUAACAACCUGCAACGGUGGCACCGAGUCGGUG | |
| | | C | |
| 334 | Yes | GUUUUAGAGCCAAAAAUGGCCAGUUAAAAUACGGCAAG | 496 |
| | | UCCAUUAGCAACAUGCACACGUGGCACCGAGUCGGUGC | |
| 335 | Yes | GUUUUAAAGCACAAAAUUGCGAGUUAAAAUAAGCCUAG | 497 |
| | | CUCGUUAUCAACAUGAACCUGUGGCACCGAGUCGGUGC | |
| 336 | Yes | GUUUAAUAGCGAGUAAUCGCAUGUUUAAAUAAGGCUA | 498 |
| | | GACCGGUAACAAAUUGAAUCAGUGGCACCGAGUCGGUG | |
| | | C | |
| 337 | Yes | GUUUUAGGUCUAGAAAUAGCGAGUUAAAAUAAGGACA | 499 |
| | | CUCCGUACGCAACGGCAAAACGUGGCACCGAGUCGGUG | |
| | | C | |
| 338 | Yes | GUUUUAGACCUAGAAAUAGCAAGUUAAAAUAACGCUGG | 500 |
| | | UCCGUUAGGAACUUCAUUCCGUGGCACCGAGUCGGUGC | |
| 339 | | GUUUUCGAGCUAGAAAUAGUAUGUGAAAAAUCGGCUA | 501 |
| | | GUACGGUAUCUACGUUAAGUAGUGGCACCGAGUCGGUG | |
| | | C | |
| 340 | Yes | GUUUUAGAGCUGGAAAAGGCAAGUUAAAAAAGGGCUA | 502 |
| | | GUCCGCAAUCAACAUGAAAACGUGGCACCGAGUCGGUG | |
| | | C | |
| 341 | | GUUUUAGAGCUAGUAAUAGCCAGUUAAAAUAAGUCUG | 503 |
| | | UUCCGUAAUCCACAUGAUUACGUGGCACCGAGUCGGUG | |
| | | C | |
| 342 | | GUUUUACAGAUUGACAUAGCAAGUUAAAACACUGCACG | 504 |
| | | CCCGUUCUCGACUUGUAAACGUGGCACCGAGUCGGUGC | |
| 343 | Yes | AUUUUAUAGUUAGAGACAACAAGUUAAAAUAAGGCUA | 505 |
| | | GUCCGUUACCAACGUGAACAUGGGGCACCGAGUCGGUG | |
| | | C | |
| 344 | Yes | GUUUUAGAGCCAGAAAUGACAAGUUAAAAUAAGGCUA | 506 |
| | | GUCCGCAUUCGACGUGGCAGUGUGGCACCGAGUCGGUG | |
| | | C | |
| 345 | Yes | GUUUUAGAGGUAGUACUACCAAGUUAAAAUAAAGCUA | 507 |
| | | GUCCGUCAACAACAUACAAACGUGGCACCGAGUCGGUG | |
| | | C | |
| 346 | Yes | GUUUUAGAGCGCUGAAGCGUCAGUUAAAAUAAAGCUAG | 508 |
| | | UCCGUUCACAACUUGGCAUAGUGGCACCGAGUCGGUGC | |
| 347 | Yes | AUUUUAGUGCUUGUAAUAGCAAGUUGAAAUAAGGCUA | 509 |
| | | GUCCGUGAACCACCUGAAACGGGGGCACCGAGUCGGUG | |
| | | C | |
| 348 | | GUUUAAGAGCCAAAAAUCGGAAUUUAAAAUAAGGCCAG | 510 |
| | | GCCGGAAUCGUCUAGAAAGAGCGGCACCGAGUCGGUGC | |
| 349 | | GUUUUAGAGCUAGAAAUAGCACGUUAAAAUAAGUCAG | 511 |
| | | GGCCGGUAUCACGUAGUAAGUGUGGCACCGAGUCGGUG | |
| | | C | |
| 350 | Yes | GUUUUAGAGCUAGAAAUAGCCUGUUAAAAUAAGGCUA | 512 |
| | | GAGUGUUACCACCAUGAAGAUGUGGCACCGAGUCGGUG | |
| | | C | |
| 351 | Yes | GUUUUACCGCUAGAAAUAGCAAGUUAAAAUAAGGCUAG | 513 |
| | | ACCGGAAUAACCAUGCAAAUGUGGCACCGAGUCGGUGC | |
| 352 | | GUUUUAUCGCUGGCAACAUCAAGUUAAAAUAACGCUAC | 514 |
| | | UUUCGGGUCACCGUGAACAGGGGGCACCGAGUCGGUGC | |
| 353 | | GUUUCAGCGCGGGCAAGGUCAAGGUAAAAUAAGUCUAG | 515 |
| | | AUCGGUAUCCACAAUUCCCCCUCGCACCGAGUCGGUGC | |
| 354 | Yes | GUUUAAUAGCGAGUAAUCGCAUGUUUAAAUAAGGCUA | 516 |
| | | GACCGGUAACAGAUUGAAUCAGUGGCACCGAGUCGGUG | |
| | | C | |
| 355 | | GUUUUAGAGCGUGUAAGCGCAAGUUAAAAUCUACCUUG | 517 |
| | | GCCGCUAACGACAUGCCGCGGCGGCACCGAGUCGGUGC | |
| 356 | Yes | GUUUUAUAGCUAGAAAUAGCAAGUUAAAAUAAGGCAA | 518 |
| | | AUCCGCUACCAAAAGCAGGCUGUGGCACCGAGUCGGUG | |
| | | C | |
| 357 | Yes | GUUUUAGCGCUAGUAAUAGCAAGUUGAAAUAAGGAUA | 519 |
| | | AUCCGUUACCAUCUGUGCACAGUGGCACCGAGUCGGUG | |
| | | C | |
| 358 | Yes | GUUUUAGAGCUAGAAAUAGCAAGUUAAAAUAAGACAA | 520 |
| | | GUACAGCAAUACCGUUAAAUCGUGGCACCGAGUCGGUG | |
| | | C | |
| 359 | | GUUUUACAGCUAGAAAUUUCAAGUGAAAAUAAGACAA | 521 |
| | | UUACGGGUGGCGCUGGAAACAGUGGCACCGAGUCGGUG | |
| | | C | |
| 360 | Yes | GUUUUAGUGCUCGAAAGAGAAAGUUAAAAUAAGAACA | 522 |
| | | UUUCGCGAUCACCGUUGAUACGUGGCACCGAGUCGGUG | |
| | | C | |
| 361 | Yes | GUUUUAGGUCUAGAAAUAGCGAGUUAAAAUAAGGACA | 523 |
| | | AUCCGUACGCAACGGCAAAACGUGGCACCGAGUCGGUG | |
| | | C | |
| 362 | Yes | GUUUUAGAGCUAGAAAUAGCAAGUUGAAAUAGGGCUU | 524 |
| | | UACCAUGCGCACCGUGAAAACGUGGCACCGAGUCGGUG | |
| | | C | |
| 363 | | GCUUUGGAGCCCUUAUUUGCUAGUUAAAAUAAGGCUCG | 525 |
| | | UCGGUUCCAACCGUGAACACGGGGCACCGAGUCGGUGC | |
| 364 | | CUUUUCGAUCAGAAAAUUGCAAGCUAAAAUAAGGUUCG | 526 |
| | | GACGUCAACAACCGUGACACGGGGCACCGAGUCGGUGC | |
| 365 | Yes | GUUUUGCAGCUAGAAUUAGCAUGUCAAAAUAAGGUUCC | 527 |
| | | UCCGGUGACAACGUGAAUACGUGGCACCGAGUCGGUGC | |
| 366 | | GUUUUAGAGCUAGAAAUAGUAAGUUAAAACAACGCAA | 528 |
| | | GUGGCAUUGUUACUUGAACCCGUGGCACCGAGUCGGUG | |
| | | C | |
| 367 | | GUUUCAGUGCUAGAAAUAGCAAGUUGAAAUAGAGCACU | 529 |
| | | AGGGUUAUCACCUACUGCCCCUGGCACCGAGUCGGUGC | |
| 368 | | UUUGACUACUGGGAUCUACAAAGUUCAAAUAAGGCCAC | 530 |
| | | UUCGUUGCCUACAAGAACGUGUGGCACCGAGUCGGUGC | |
| 369 | Yes | UUUUCAGUGCUAGAAUUAGCAAGUUGAAAUAAGGUUA | 531 |
| | | UUCCGUGCCUGCCUGGACAGGGUGGCACCGAGUCGGUG | |
| | | C | |
| 370 | | GUUUAAGAGUUUAACACAACAAGUUUAAAUACCGAUAU | 532 |
| | | UGGCAUCUACCCAGGACACGGUGGCACCGAGUCGGUGC | |
| 371 | | ACUUGCUGACAUCCCUGCCGAAGUUUUAAUAACGAUAG | 533 |
| | | UCUGUUACCCACCAGAAACAGUGGCACCGAGUCGGUGC | |
| 372 | | GUUUUUCUGACCACCUGGUUAAGUAAAAAUAUCGGUAG | 534 |
| | | UCUGACGUCCGCUGGCCGGGGCGGCACCGAGUCGGUGC | |
| 373 | Yes | GUUUGAGAGCUAAAAAUAGCAAGUUCAAAUAAGGUUA | 535 |
| | | GACCGUAAUUUCGUUGUACAUGUGGCACCGAGUCGGUG | |
| | | C | |
| 374 | | GUUUGAGAGCUAGAAAAAGCAAGUUCAAAUAAUGUAA | 536 |
| | | GUCGGUUAUCGCCAGAAACCUGUAGCACCGAGUCGGUG | |
| | | C | |
| 375 | Yes | UAUUUAGAGGUCGAAAAACCAAGUUAAAAUAAGGUUA | 537 |
| | | AACCGUUAUAACCUGGAACAGUUGGCACCGAGUCGGUG | |
| | | C | |
| 376 | | GUCUAAAUCCUUGUAAUCGCUUGUCAAAAUAAGGAUGG | 538 |
| | | UUCAAUCGGACCCACAAACCGUGGCACCGAGUCGGUGC | |
| 377 | | GUCUUAUACACAGAUUCGCCCAGUGCAAAUAAGGCUAC | 539 |
| | | GCCGCUCUGACCCGCAACAGGGGGCACCGAGUCGGUGC | |
| 378 | | GCUUUAAAGAUCGACAACCUCGAAGCAAAUAAGGCAAG | 540 |
| | | AUCCUGCCUAUCUUGAAAAGGUGGCACCGAGUCGGUGC | |
| 379 | | GUAUUAGAGCUCCAAAGAGCAAGUUAAAAUCAGGCUAG | 541 |
| | | GUGGUUAACGACCGCAUACUGUGGCACCGAGUCGGUGC | |
| 380 | | GUUUUGGAGCUGGAAACAGCAAGUUUGAAGAAGGCGU | 542 |
| | | GUCUGCUGGCAACCUGACAGGGCGGCACCGAGUCGGUG | |
| | | C | |
| 381 | Yes | GUUUUAGUGCUGUAAUCAGCGAGUUAAAAUGAGGCAA | 543 |
| | | UUCUGUUAUCAACCUGUAAUGGUGGCACCGAGUCGGUG | |
| | | C | |
| 382 | | GAUUUGGACCUAGAGAUGCCAAAUCAAAAUAACGAGAG | 544 |
| | | UUCGUUAUCAAGGUGCCAAGGUGGCACCGAGUCGGUGC | |
| 383 | | GUUUUAGAGCUAACCAAAGCACGUUAAAGUAACGUUAA | 545 |
| | | UUCGCUCUUAAUGUGGAACCGUGGCACCGAGUCGGUGC | |
| 384 | | GUUUUAACGCUAGUUAUAGCAUGUUCAAAUAAGGUGA | 546 |
| | | GUACGCGAUUAAGUGGCUGGUGUGGCACCGAGUCGGUG | |
| | | C | |

### Example 3—Staphylococcus aureus Cas9 (saCas9) Guide RNA Scaffold Evolution for Understanding of Structure-Function Relationships and Improvement of Targeting Efficiency

Sp Cas9, derived from Streptococcus pyogenes, is currently the most utilized CRISPR system (11). Unfortunately, due to the large size of the spCAS9 protein, this system could not be packaged into a single AAV vector, the leading gene therapy approach. This led to a search for CRISPR-CAS9 systems in other bacterial species and the discovery of CRISPR S. aureus Cas9 CRISPR-saCAS9. With a smaller CAS9 protein, this is the leading ortholog used in gene therapy (12, 13).

These systems consist of a single guide RNA (sgRNA) and the Cas9 protein coming together to form a DNA targeting/cleaving/editing capable Ribonucleoprotein (RNP) complex (1). The sgRNA consists of a ˜20 nucleotide variable “targeting region” responsible for targeting DNA for editing, followed by an 80 nucleotide “scaffold region” allowing binding and functional activation of the Cas9 protein (1,14).

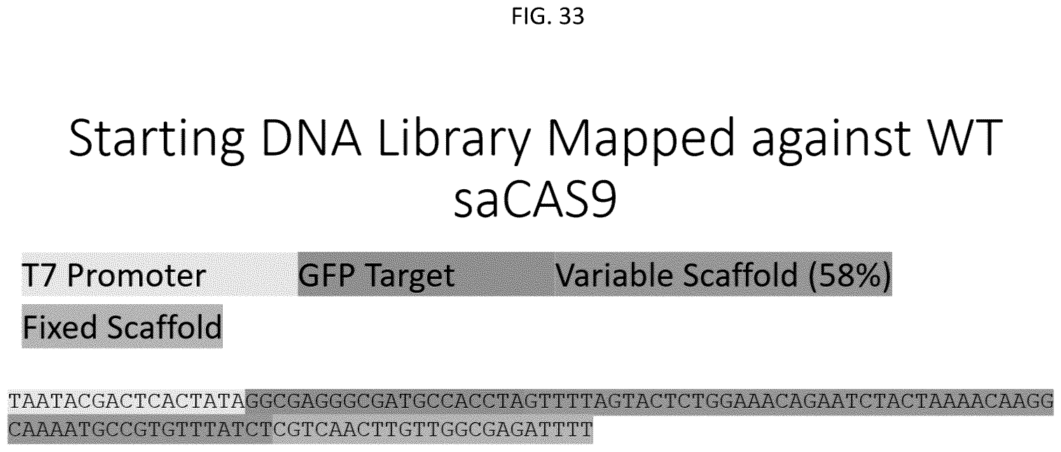

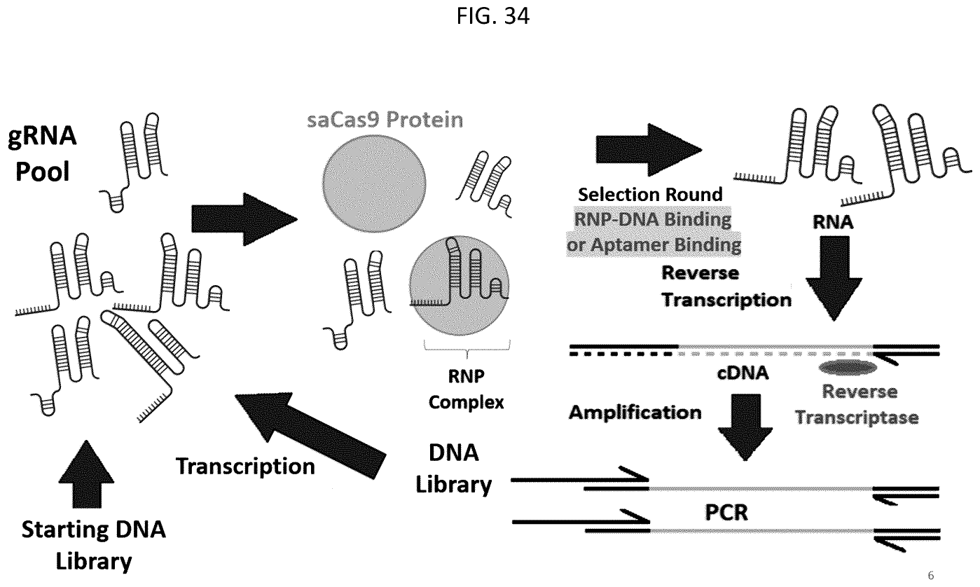

Current engineering efforts have focused on CAS9 protein mutagenesis, leading to improvements for on-target efficiency and reduced off-target effects (13,15). sgRNA variant exploration, on the other hand, has remained largely limited to few variants resulting from rational design (14,16), and improved variants utilizing chemical modification (17-19), which remains expensive for large scale studies. The inventors used a high throughput selection method for functional sgRNA scaffolds utilizing Systematic evolution of ligands by exponential enrichment (SELEX). SELEX consists on the iterative binding and amplification of nucleic acid sequences where each iteration of the cycle is termed a “round” (FIG. 34), allowing enrichment of molecules possible from massively diverse libraries (20).

Unfortunately, CRISPR-Cas9 targeting regions are not created equal, displaying a wide range of DNA cleaving activities (4-8), forcing researchers to spend time testing a multitude of sgRNA target candidates for applications as simple as gene knockout (9,10) More advanced editing modalities involving homologous directed repair, widely used in the generation of animal models, cell lines and therapeutics, suffer far more from inefficient site targeting with targeting efficiency most often determining the success of a gene editing project (21). Making it difficult to progress in precision editing projects based around an inherently low efficiency CRISPR-CAS9 target.

Therefore, the prediction as well as improvement of editing efficiency has been a central topic of CRISPR research (4,5,22). Predictive algorithms have succeeded in correlating editing efficiency to Cas9/sgRNA-DNA complex binding stability (23), with significant editing improvement at some difficult sites achieved through the stabilization of known sgRNA secondary structure features of the sgRNA scaffold by chemical modification (24). Despite this progress, chemically modifying the sgRNA remains prohibitively expensive due to low synthesis efficiency for RNAs with more than 60 bases (25). On the other hand, rational design has still only explored a small fraction of available sequence space within the sgRNA scaffold and therefore has strong potential as an unexplored area of CRISPR biology.

### Experimental Design and Methods

Starting DNA Library Generation

The inventors initially generated a DNA library utilizing the same parameters as the original spCas9 selection, namely the inventors synthesized a DNA library where, the first 40 nucleotides of sequence were fixed, and corresponded to the targeting region preceded by the T7 RNA polymerase promoter. The next 60 nucleotides of sequence were synthesized in a “doped” library fashion, where each nucleotide position maintained a 58% probability of staying true to its wildtype saCAS9 sequence, and a 14% probability of being either of the other 3 possible nucleotides, for a 42% probability of deviating at each position overall. Lastly the final 20 nucleotides of sequence synthesized were once again 100% fixed and this time corresponded to the last stem loop of the saCAS9 sgRNA. The fixed ends of sequence of this design allow for polymerase chain reaction (PCR) amplification at the end of each round. The inventors used the “doped” selection parameters for the variable region as the inventors were unsure if saCAS9 would tolerate significant deviations from its original sequence.

Next to convert the ssDNA pool to dsDNA the inventors set up an annealing reaction by adding 10 ul of 10×NEB Buffer 2, 0.5 nmole library and 1 nmole of C9.deg.Forward primer and then adding nuclease free water to 100 ul. This was followed by incubation at 90° C. for 3 minutes followed by cooling at 25° C. for 10′. Next, the inventors set up Klenow reaction by taking the annealing reaction and adding 10 ul of NEB Buffer 2, 4 ul of 25 mM dNTP and 57 ul of nuclease free water and adding 30 units of NEB Exo-Klenow enzyme. The mixture was incubated at 37° C. for 1.5 hrs and heated to 75 degrees for 20 minutes to inactivate the enzyme.

SELEX Process

6 Rounds of SELEX were carried out consisting of the following steps:
1. RNA Transcription: The inventors used NEB T7 RNA polymerase following manufacturing protocol scaled up 40× for round 0 RNA generation and 20× thereafter. Namely the 1× protocol using 2 ul 10×RNA polymerase buffer, 0.5 mM NTPs, 1 ug of Template DNA, 5 mM fresh DTT and 2 ul of T7 RNA polymerase.
2. Purification: Transcriptions were run on a 12% denaturing Urea acrylamide gel and gel extracted by UV shadowing. Gel fragments were incubated overnight in TE buffer, filtered through a 200 micron filter and further purified using an Amicon Ultra 15 3 Kda filter.
3. RNP-DNA Binding Selection:
   a. RNP formation: For all rounds 20 ug RNA pools (621.8 μmol) were incubated with 62 pmol NEB Engen SaCAS9 for 30 minutes at room temperature with gentle rotation in NEB buffer 3.1 (100 mM NaCl, 50 mM Tris-HCl, 10 mM MgCl2, 100 μg/ml BSA) with pH adjusted to 8.4.
   b. Generating the DNA reagent: A 656 bp synthesized piece of DNA bearing the saCAS9 target region and PAM sequence was PCR amplified using a 5′ biotinilated reverse primer (corresponding to the PAM proximal end of the DNA molecule) and an unlabeled forward primer.
   c. RNP-DNA Complex Formation: 30 pmol of PAM proximal biotin labeled DNA reagent was added to the RNP formation mix and incubated for 1 hour at 37 degrees.
   d. RNP-DNA Magnetic Complex Pulldown: 1 ul of Thermofisher MyOne C1 1 micron beads bearing streptavidin were added to the RNP-DNA Complex mixture and incubated with rotation for 15 minutes at room temperature to allow binding to biotin. Subsequently utilizing a magnet, beads were pelleted and the RNP-DNA complex mix was discarded. Beads were resuspended with the ph modified NEB buffer 3.1 described in 3a, addition of final 0.006% Tween 20 detergent concentration, and re-pelleted down with a magnet. This wash step was repeated 5 times.
   e. Nucleic acid Purification from Magnetic Beads: Pelleted beads were resuspended in 800 ul of Phenol-Cholorform-Isoamyl solution (Thermo cat 15593031), vortexed vigorously, and spun down at 21000 RCF for 5 minutes. The phase containing RNA and DNA was collected and ethanol precipitated using linear acrylamide. DNAse was used to break down DNA in the sample and once again Phenol Chloroform extracted and ethanol precipitated.
4. Reverse Transcription and Amplification: Samples were reverse transcribed using MMLV reverse transcriptase per manufacturer protocol. Then samples were PCR amplified using a forward primer (complementary to the targeting region preceded by a T7 polymerase promoter) and a reverse primer complementary to the fixed saCAS9 scaffold region using platinum Taq polymerase per manufacturer protocols.

The samples were analyzed by Next Generation Sequencing: DNA library Samples were sent to Genewiz where library preparation was carried out prior to sequencing. Data was preprocessed for high sequencing quality using useGalaxy.org and subsequently analyzed in R.

Cellular Genome Editing Assays

Next, the inventors tested the in vitro active sgRNAs from process 1 in HEK293 cells. 14000 Cells were plated per well in 96 well plates 24 hours prior to transfection. This was carried out via Lipofectamine 2000 transfection of RNP complexes. Briefly 1 picomole of sgRNA and Cas9 protein were complexed for 10 minutes at room temperature in 25 μl of Optimem medium, then 111.1 of lipofectamine 2000 was added to the mix and incubated for 20 minutes prior to addition to each well. GFP Knockdown was measured via flow cytometry 5 days post transfection to eliminate noncleaving sgRNA silencing effects. For the top guides performing best in the knockdown assays, genomic DNA was extracted from >100000 cells, and a 1000 bp fragment around the expected cut site was amplified via PCR and submitted for Sanger Sequencing. Utilizing Synthego's ICE algorithm (35), trace decomposition analysis of the trace files compared to an unedited control genome trace file was carried out to estimate genome editing efficiency.

### Results

The inventors initially generated a DNA library utilizing the same parameters as the spCas9 selection described in Example 1 and 2, namely the inventors synthesized a DNA library where the first 40 nucleotides of sequence were fixed and corresponded to the targeting region preceded by the T7 RNA polymerase promoter. The next 60 nucleotides of sequence were synthesized in a “doped” library fashion, where each nucleotide position maintained a 58% probability of staying true to its wildtype saCAS9 sequence, and a 14% probability of being either of the other 3 possible nucleotides, for a 42% probability of deviating at each position overall. Lastly the final 20 nucleotides of sequence synthesized were once again 100% fixed and this time corresponded to the last stem loop of the saCAS9 sgRNA (FIG. 23A). The fixed ends of sequence of this design allow for polymerase chain reaction (PCR) amplification at the end of each round. The inventors used the “doped” selection parameters for the inner region as the inventors were unsure if saCAS9 would tolerate significant deviations from its original sequence as spCAS9. Furthermore, the inventors also followed this method to reduce the possible sequence space and so that the inventors could cover a larger percentage of it with the size of the synthesized pool.

The Starting DNA library was used to transcribe an RNA library which was taken through the SELEX process with either RNP-DNA binding or Aptamer Binding rounds (FIG. 34). The RNA that was recovered was subjected to reverse transcription to cDNA using Moroney murine leukemia virus (MMLV) Reverse transcriptase. This cDNA was then PCR amplified using primers containing the T7 promoter and binding to the constant ends, effectively reforming a DNA library that could be transcribed once again to RNA (FIG. 34).

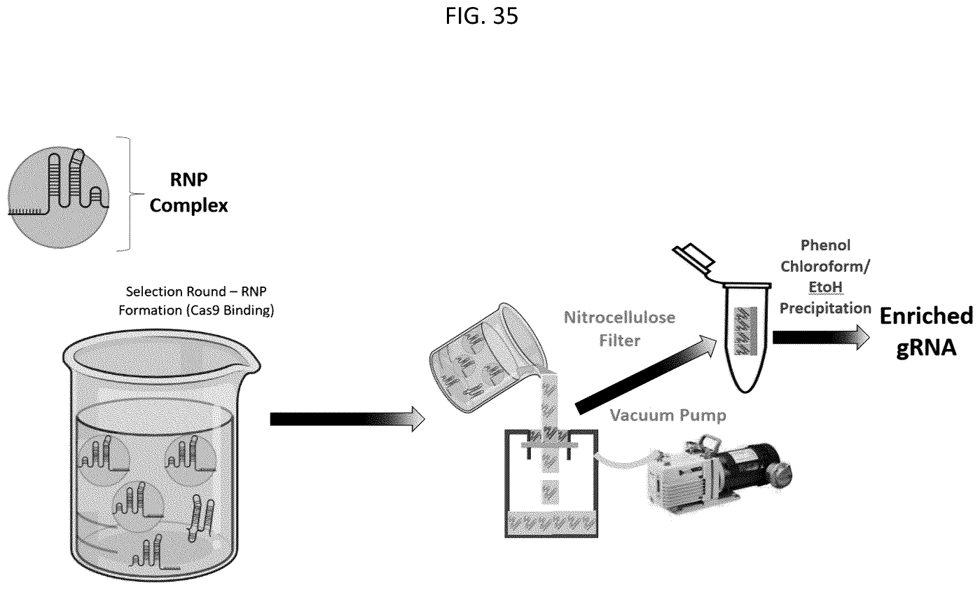

Aptamer binding relies on the classic aptamer approach of RNA binding to protein to form RNPs, then these are passed through a Nitrocellulose filter and washed, finally the inventors extract the RNA from the nitrocellulose with Phenol chloroform extraction and ethanol precipitation, after which the inventors can proceed to reverse transcription step from the previous SELEX slide (FIG. 35).

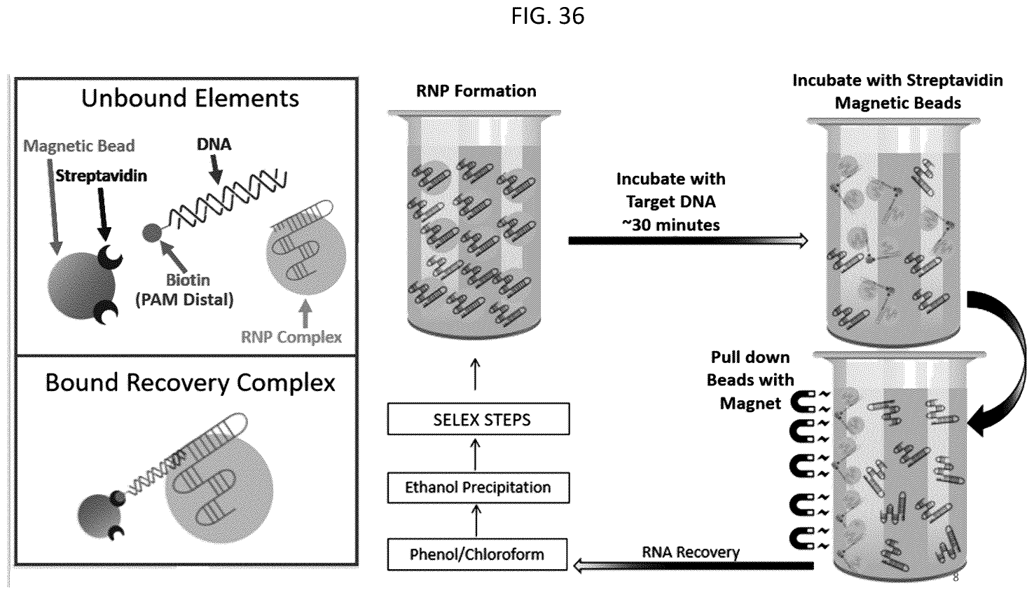

The method that ended up being used exclusively however was RNP-DNA binding, where the inventors generate DNA fragments with Biotin on either the Proximal or distal end. The biotin will bind strongly to Magnetic beads that are coated with streptavidin. This DNA strand will harbor the cutsite for the saCAS9, and based on the binding of saCAS9 RNP to the DNA, it will allow us to pull down the protein alongside with the guide RNA that is bound to that protein (FIG. 36). Interestingly, the literature indicated pulling DNA from the PAM proximal instead of PAM distal is mechanistically more likely to include cleaving saCAS9 variants.

The inventors tested how much 32 P radiolabeled gRNA was pulled down using the RNP-DNA binding assay: the inventors formed RNPs using saCAS9 protein with either WT gRNA or our unenriched pool gRNA (where the inventors expect the pool to be mostly non-binding) and incubated them with target DNA with a biotin attached to either the proximal end exclusively or the distal end of the DNA strand. The inventors proceeded to incubate this with magnetic beads bearing streptavidin and use a magnet to pull down the complexes. The inventors observed the PAM proximal biotin labeled DNA had higher pull-down percentages and that a middle level of detergent was optimal for PAM proximal pull down (FIG. 37).

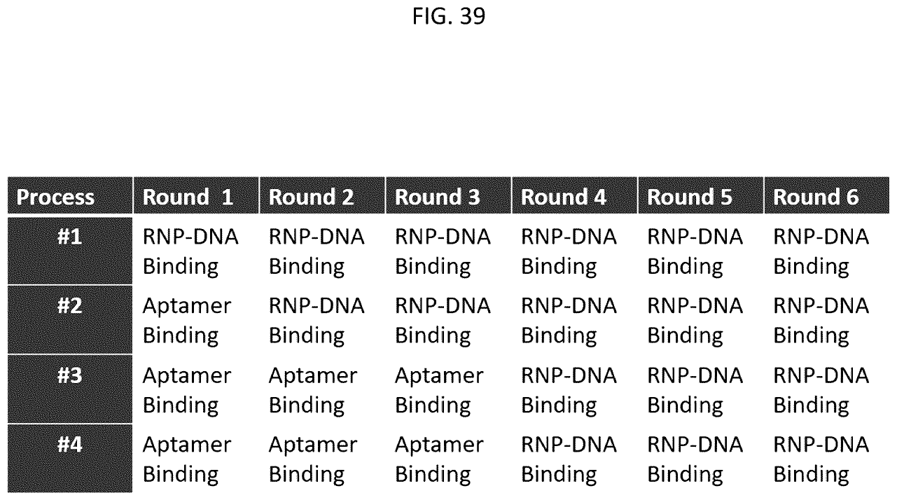

The inventors tested various processes combining aptamer binding or RNP-DNA binding rounds (where process #4 had a targeting region with an extra G at the end that produced better initial WT cleaving). Ultimately process 1 was the only one to yield functional variant gRNA (FIG. 39).

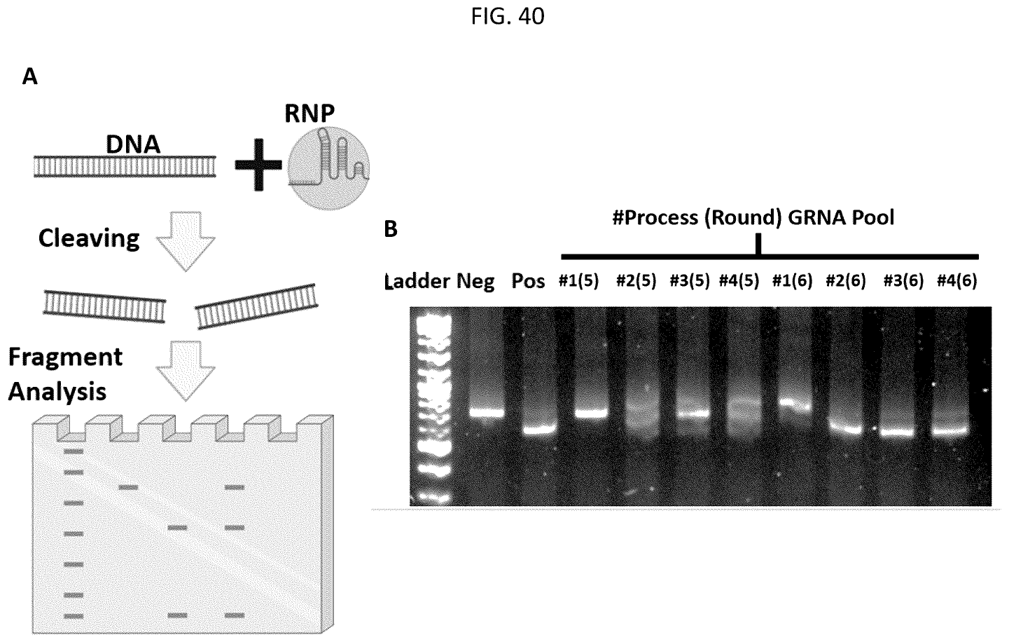

After 5-6 rounds, all processes produced in vitro cleaving pools when complexed to saCAS9 RNPs (FIG. 40B). Note process 1 was the only one that ultimately lead to the generation of variant sequences. In the other processes, wildtype-like sequences likely were responsible for the cleaving activity observed.

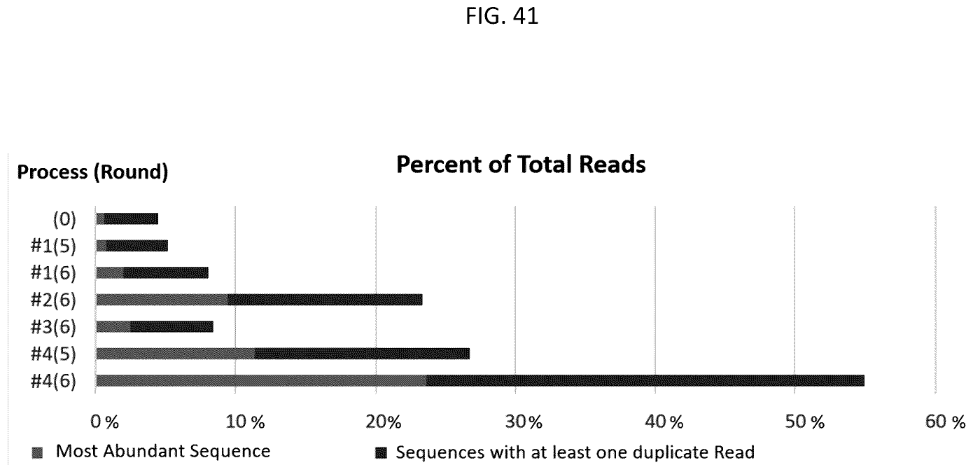

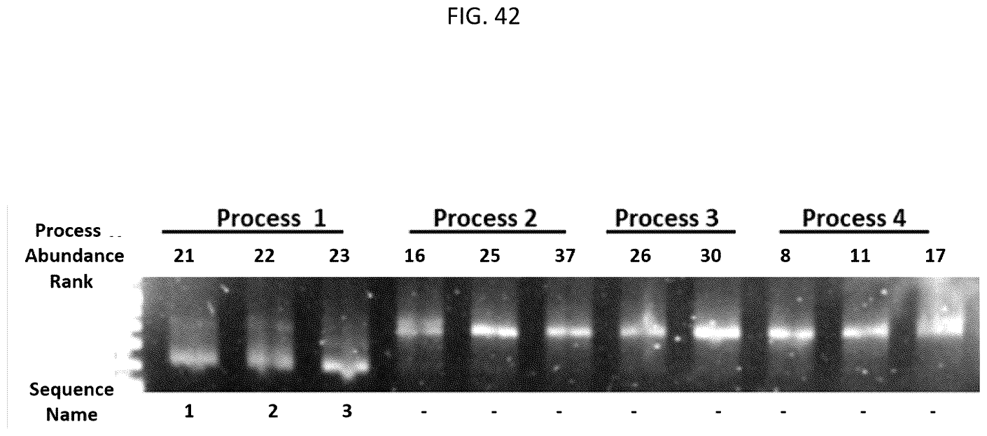

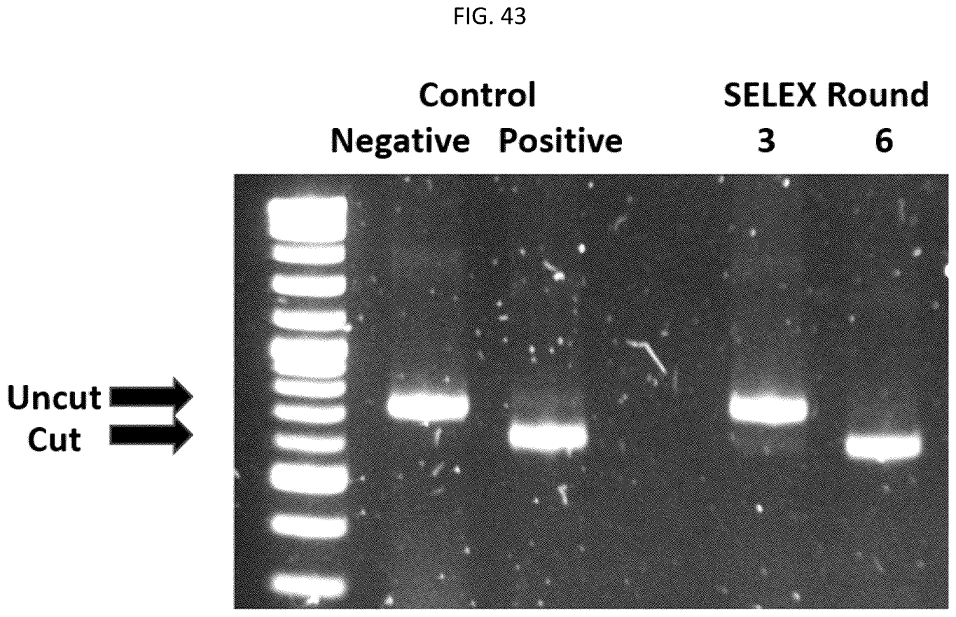

All processes were enriched relative to round 0 in most abundant sequence # of total reads and % of sequences that had at least one duplicate read by next generation sequencing (FIG. 41). Testing gRNAs individually for cleaving, the inventors selected gRNAs that had at least 10% mutations, i.e., greater than 8 mutations, relative to the entire 80 nucleotide gRNA scaffold. Only process 1 variants were seen to produce strong cleaving of DNA (FIG. 42). The results of Process 1 were examined between rounds 3 and 6 and the inventors noted improved cleaving activity of the pool in the 6th round (FIG. 43).

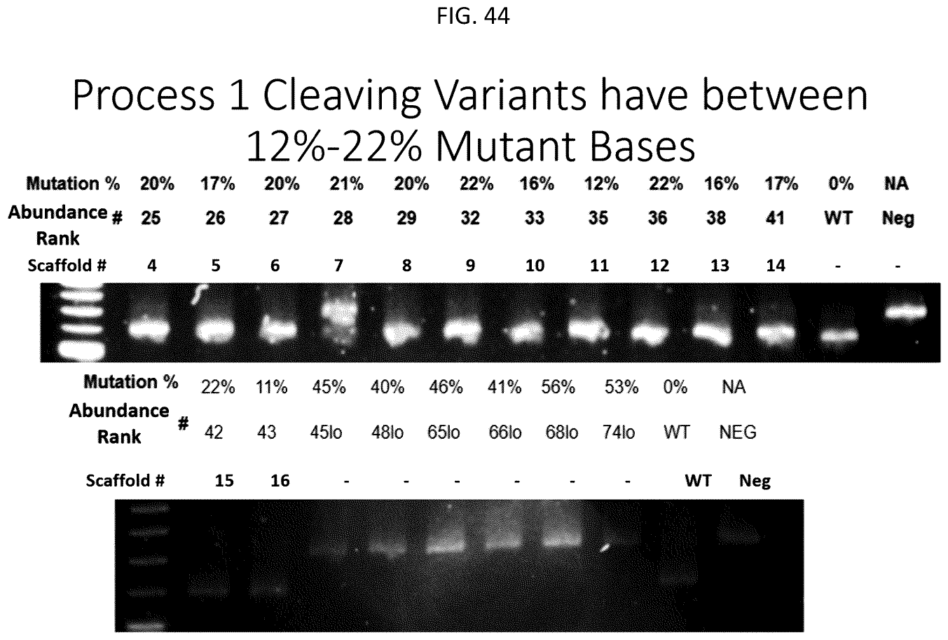

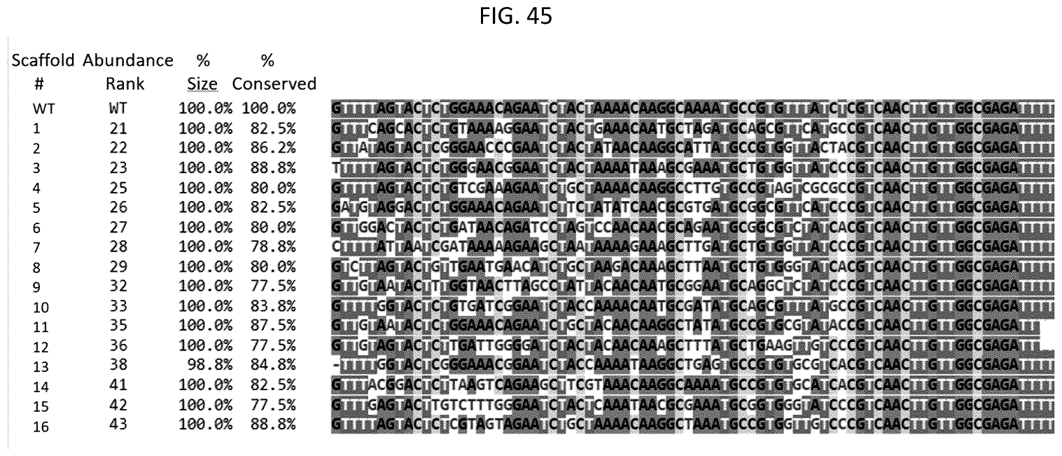

The inventors tested other variant sequences with between 12% and 22% mutations and observed near complete cleaving for most samples. The inventors tested gRNAs with more mutations irrespective of their abundance ranks in the selection and observed these high mutation (40-56% mutation) were unable to cleave (FIGS. 44 and 45).

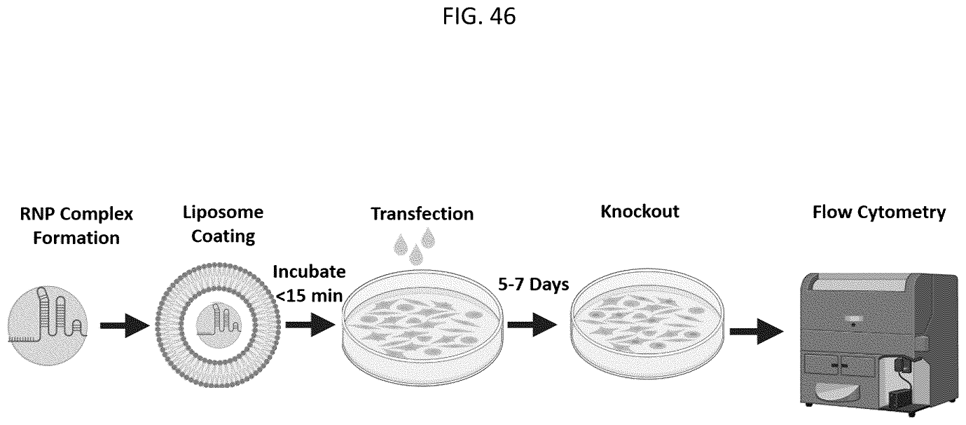

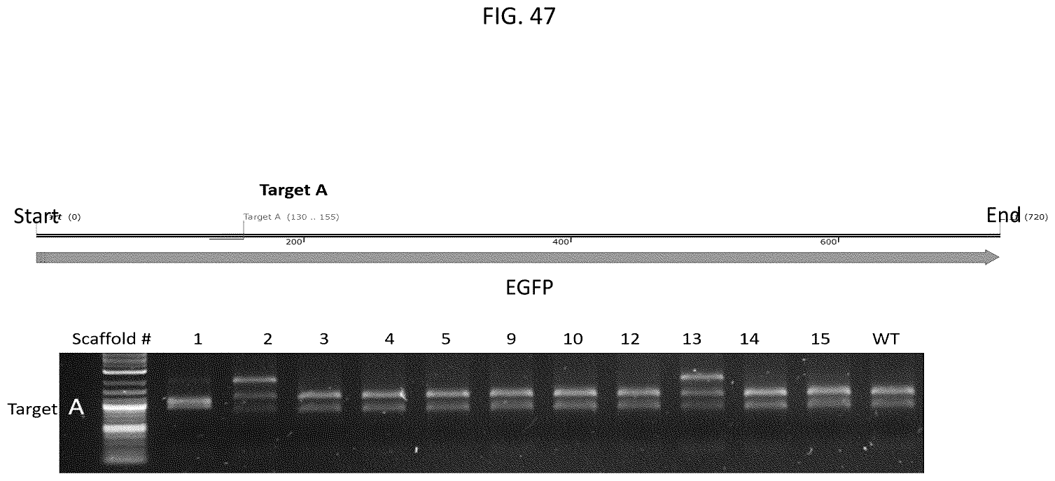

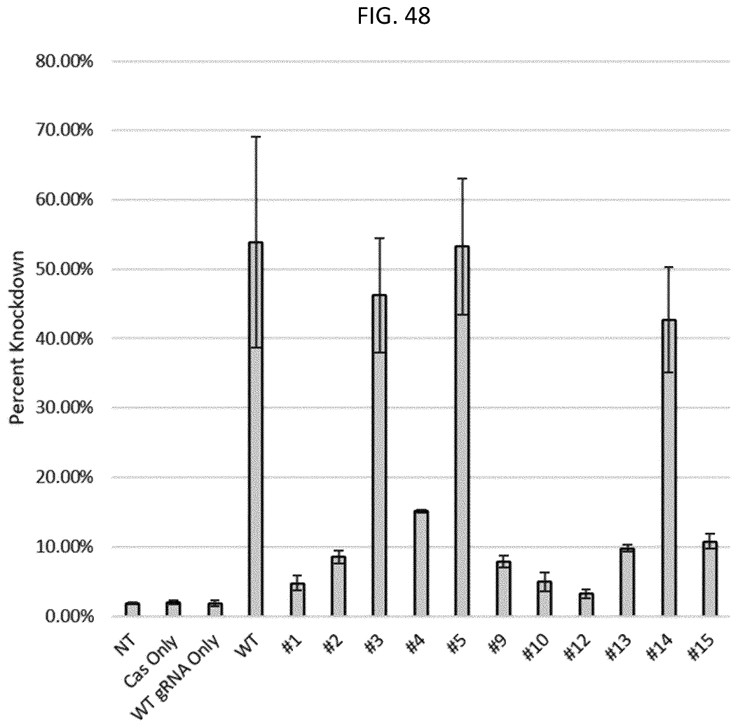

Novel gRNA scaffolds 1 and 2-15 were capable of cleaving target DNA in vitro (FIG. 47). When the novel gRNAs were introduced into a cell expressing target DNA (GFP) using the method shown in FIG. 46, gRNAs 3, 5, and 14 showed comparable knockdown levels to the wild type gRNA (FIG. 48).

In summary, the inventors generated variant saCas9 gRNA scaffolds with the capacity for targeting saCas9 for cleavage using process 1. Importantly, the selection of saCas9 gRNAs only utilized a binding step to select for variants. By contrast, selection of functional spCas9 variant gRNAs required selection of variants gRNAs capable of binding to spCas9 and catalyzing cleavage using the novel TdT selection method, as selection by binding produced no functional spCas9 gRNA variants.

TABLE 3
WT and novel variant S. aureus gRNAs. See FIG. 47-48.

| Sa | | | SEQ | | |
|---|---|---|---|---|---|
| Scaffold | Abundance | | ID | In vitro | In cell |
| # | Rank | Sequence | NO: | activity | activity |
| wt | NA | GTTTTAGTACTCTGGAAACAGAATCTAC | 547 | >90% | >50% |
| | | TAAAACAAGGCAAAATGCCGTGTTTATC | | | |
| | | TCGTCAACTTGTTGGCGAGATTTT | | | |
| 1 | 21 | GTTTCAGCACTCTGTAAAAGGAATCTAC | 548 | >90% | <10% |
| | | TGAAACAATGCTAGATGCAGCGTTCATG | | | |
| | | CCGTCAACTTGTTGGCGAGATTTT | | | |
| 2 | 22 | GTTATAGTACTCGGGAACCCGAATCTAC | 549 | >95% | <10% |
| | | TATAACAAGGCATTATGCCGTGGTTACT | | | |
| | | ACGTCAACTTGTTGGCGAGATTTT | | | |
| 3 | 23 | TTTTTAGTACTCTGGGAACGGAATCTAC | 550 | >95% | >40% |
| | | TAAAATAAAGCGAAATGCTGTGGTTATC | | | |
| | | CCGTCAACTTGTTGGCGAGATTTT | | | |
| 4 | 25 | GTTTTAGTACTCTGTCGAAAGAATCTGC | 551 | >95% | >10% |
| | | TAAAACAAGGCCTTGTGCCGTAGTCGCG | | | |
| | | CCGTCAACTTGTTGGCGAGATTTT | | | |
| 5 | 26 | GATGTAGGACTCTGGAAACAGAATCTTC | 552 | >95% | >50% |
| | | TATATCAACGCGTGATGCGGCGTTCATC | | | |
| | | CCGTCAACTTGTTGGCGAGATTTT | | | |
| 6 | 27 | GTTGGACTACTCTGATAACAGATCCTAG | 553 | >95% | N/A |
| | | TCCAACAACGCAGAATGCGGCGTCTATC | | | |
| | | ACGTCAACTTGTTGGCGAGATTTT | | | |
| 7 | 28 | CTTTTATTAATCGATAAAAAGAAGCTAA | 554 | <20% | N/A |
| | | TAAAAGAAAGCTTGATGCTGTGGTTATC | | | |
| | | CCGTCAACTTGTTGGCGAGATTTT | | | |
| 8 | 29 | GTCTTAGTACTGTTGAATGAACATCTGC | 555 | >95% | N/A |
| | | TAAGACAAAGCTTAATGCTGTGGGTATC | | | |
| | | ACGTCAACTTGTTGGCGAGATTTT | | | |
| 9 | 32 | GTTGTAATACTTTGGTAACTTAGCCTATT | 556 | >95% | <10% |
| | | ACAACAATGCGGAATGCAGGCTCTATCC | | | |
| | | CGTCAACTTGTTGGCGAGATTTT | | | |
| 10 | 33 | GTTTTGGTACTCTGTGATCGGAATCTACC | 557 | >95% | <10% |
| | | AAAACAATGCGATATGCAGCGTTTATGC | | | |
| | | CGTCAACTTGTTGGCGAGATTTT | | | |
| 11 | 35 | GTTGTAATACTCTGGAAACAGAATCTGC | 558 | >95% | N/A |
| | | TACAACAAGGCTATATGCCGTGCGTATA | | | |
| | | CCGTCAACTTGTTGGCGAGATTTT | | | |
| 12 | 36 | GTTGTAGTACTCTTGATTGGGGATCTACT | 559 | >95% | <10% |
| | | ACAACAAAGCTTTATGCTGAAGTTGTCC | | | |
| | | CGTCAACTTGTTGGCGAGATTTT | | | |
| 13 | 38 | TTTTGGTACTCGGGAAACGGAATCTACC | 560 | >95% | <10% |
| | | AAAATAAGGCTGAGTGCCGTGTGCGTCA | | | |
| | | CGTCAACTTGTTGGCGAGATTTT | | | |
| 14 | 41 | GTTTACGGACTCTTAAGTCAGAAGCTTC | 561 | >95% | >40% |
| | | GTAAACAAGGCAAAATGCCGTGTGCATC | | | |
| | | ACGTCAACTTGTTGGCGAGATTTT | | | |
| 15 | 42 | GTTTGAGTACTTGTCTTTGGGAATCTACT | 562 | >95% | >10% |
| | | CAAATAACGCGAAATGCGGTGGGTATCC | | | |
| | | CGTCAACTTGTTGGCGAGATTTT | | | |
| 16 | 43 | GTTTTAGTACTCTCGTAGTAGAATCTGCT | 563 | >95% | N/A |
| | | AAAACAAGGCTAAATGCCGTGGTTGTCC | | | |
| | | CGTCAACTTGTTGGCGAGATTTT | | | |

### References for Example 3

1. Jinek, Martin, et al. “A Programmable Dual-RNA—Guided DNA Endonuclease in Adaptive Bacterial Immunity.” Science, vol. 337, no. 6096, 2012, pp. 816-821., https://doi.org/10.1126/science.1225829.
2. Adli, M. The CRISPR tool kit for genome editing and beyond. Nat Commun 9, 1911 (2018). https://doi.org/10.1038/s41467-018-04252-2
3. Brandt, Katelyn, and Rodolphe Barrangou. “Applications of CRISPR Technologies across the Food Supply Chain.” Annual Review of Food Science and Technology, vol. 10, no. 1, 2019, pp. 133-150., https://doi.org/10.1146/annurev-food-032818-121204.
4. Moreno-Mateos, Miguel A, et al. “Crisprscan: Designing Highly Efficient Sgrnas for CRISPR-Cas9 Targeting in Vivo.” Nature Methods, vol. 12, no. 10, 2015, pp. 982-988., https://doi.org/10.1038/nmeth.3543.
5. Labun, Kornel, et al. “CHOPCHOP V2: A Web Tool for the next Generation of CRISPR Genome Engineering.” Nucleic Acids Research, vol. 44, no. W1, 2016, https://doi.org/10.1093/nar/gkw398.
6. Doench, John G, et al. “Optimized Sgrna Design to Maximize Activity and Minimize off-Target Effects of CRISPR-Cas9.” Nature Biotechnology, vol. 34, no. 2, 2016, pp. 184-191., https://doi.org/10.1038/nbt.3437.
7. Doench, John G, et al. “Rational Design of Highly Active Sgrnas for CRISPR-Cas9— Mediated Gene Inactivation.” Nature Biotechnology, vol. 32, no. 12, 2014, pp. 1262-1267., https://doi.org/10.1038/nbt.3026.
8. Xu, Han, et al. “Sequence Determinants of Improved CRISPR Sgrna Design.” Genome Research, vol. 25, no. 8, 2015, pp. 1147-1157., https://doi.org/10.1101/gr.191452.115.
9. Cradick, Thomas J., et al. “CRISPR/Cas9 Systems Targeting β-Globin and CCRS Genes Have Substantial off-Target Activity.” Nucleic Acids Research, vol. 41, no. 20, 2013, pp. 9584-9592., https://doi.org/10.1093/nar/gkt714.
10. Hall B, Cho A, Limaye A. et al. Genome editing in mice using CRISPR/Cas9 technology. Curr Protoc Cell Biol 2018;81:e57
11. Xu Y., Li Z. CRISPR-Cas systems: Overview, innovations and applications in human disease research and gene therapy. Comput. Struct. Biotechnol. J. 2020; 18:2401-2415. doi: 10.1016/j.csbj.2020.08.031.
12. Uddin, Fathema, et al. “CRISPR Gene Therapy: Applications, Limitations, and Implications for the Future.” Frontiers in Oncology, vol. 10, 2020, https://doi.org/10.3389/fonc.2020.01387.
13. Tan, Yuanyan, et al. “Rationally Engineered Staphylococcus Aureus cas9 Nucleases with High Genome-Wide Specificity.” Proceedings of the National Academy of Sciences, vol. 116, no. 42, 2019, pp. 20969-20976., https://doi.org/10.1073/pnas.1906843116.
14. Nishimasu, Hiroshi, et al. “Crystal Structure of Staphylococcus Aureus Cas9.” Cell, vol. 162, no. 5, 2015, pp. 1113-26, https://doi.org/10.1016/j.ce11.2015.08.007.
15. Karthik Murugan, Shravanti K Suresh, Arun S Seetharam, Andrew J Severin, Dipali G Sashital, Systematic in vitro specificity profiling reveals nicking defects in natural and engineered CRISPR-Cas9 variants, Nucleic Acids Research, Volume 49, Issue 7, 19 Apr. 2021, Pages 4037-4053, https://doi.org/10.1093/nar/gkab163
16. Zhang, Dong, et al. “Unified Energetics Analysis Unravels SpCas9 Cleavage Activity for Optimal GRNA Design.” Proceedings of the National Academy of Sciences, vol. 116, no. 18, 2019, p. 201820523, https://doi.org/10.1073/pnas.1820523116.
17. Yin H, et al. Structure-guided chemical modification of guide RNA enables potent non-viral in vivo genome editing. Nat Biotechnol. 2017; 35(12):1179-1187. doi: 10.1038/nbt.4005.
18. Hendel, A., Bak, R., Clark, J. et al. Chemically modified guide RNAs enhance CRISPR-Cas genome editing in human primary cells. Nat Biotechnol 33, 985-989 (2015). https://doi.org/10.1038/nbt.3290
19. Chen, Qiubing, et al. “Recent Advances in Chemical Modifications of Guide RNA, Mrna and Donor Template for CRISPR-Mediated Genome Editing.” Advanced Drug Delivery Reviews, vol. 168, 2021, pp. 246-258., https://doi.org/10.1016/j.addr.2020.10.014.
20. Komarova N., Kuznetsov A. Inside the Black Box: What Makes SELEX Better? Molecules. 2019; 24:3598. doi: 10.3390/molecules24193598.
21. Xu, Kun, et al. “Editorial: Precise Genome Editing Techniques and Applications.” Frontiers in Genetics, vol. 11, 2020, https://doi.org/10.3389/fgene.2020.00412.
22. Xiang, X., Corsi, G.I., Anthon, C. et al. Enhancing CRISPR-Cas9 gRNA efficiency prediction by data integration and deep learning. Nat Commun 12, 3238 (2021). https://doi.org/10.1038/s41467-021-23576-0
23. Xu, X., Duan, D. & Chen, S J. CRISPR-Cas9 cleavage efficiency correlates strongly with target-sgRNA folding stability: from physical mechanism to off-target assessment. Sci Rep 7, 143 (2017). https://doi.org/10.1038/s41598-017-00180-1
24. Riesenberg, S., Helmbrecht, N., Kanis, P. et al. Improved gRNA secondary structures allow editing of target sites resistant to CRISPR-Cas9 cleavage. Nat Commun 13, 489 (2022). https://doi.org/10.1038/s41467-022-28137-7
25. Flamme, Marie, et al. “Chemical Methods for the Modification of RNA.” Methods, vol. 161, 2019, pp. 64-82., https://doi.org/10.1016/j.ymeth.2019.03.018.
26. Buddai, SK; Layzer, J M; Lu, G; Rusconi, CP; Sullenger, BA; Monroe, DM; Krishnaswamy, S, An anticoagulant RNA aptamer that inhibits proteinase-cofactor interactions within prothrombinase., The Journal of Biological Chemistry, vol 285 no. 8 (2010), pp. 5212-5223 [10.1074/jbc.M109.049833] [abs].
27. Wang, J; Wakeman, TP; Lathia, JD; Hjelmeland, AB; Wang, X-F; White, RR; Rich, JN; Sullenger, BA, Notch promotes radioresistance of glioma stem cells., Stem Cells, vol 28 no. 1 (2010), pp. 17-28 [10.1002/stem.261] [abs].
28. Mi, J; Liu, Y; Rabbani, ZN; Yang, Z; Urban, JH; Sullenger, BA; Clary, BM, In vivo selection of tumor-targeting RNA motifs., Nat Chem Biol, vol 6 no. 1 (2010), pp. 22-24 [10.1038/nchembio.277] [abs].
29. Oney, S; Lam, RTS; Bompiani, KM; Blake, CM; Quick, G; Heidel, JD; Liu, JY-C; Mack, BC; Davis, ME; Leong, KW; Sullenger, BA, Development of universal antidotes to control aptamer activity., Nat Med, vol 15 no. 10 (2009), pp. 1224-1228 [10.1038/nm.1990] [abs].
30. Blake, CM; Sullenger, BA; Lawrence, DA; Fortenberry, YM, Antimetastatic potential of PAI-1-specific RNA aptamers., Oligonucleotides, vol 19 no. 2 (2009), pp. 117-128 [10.1089/oli.2008.0177] [abs].
31. Long, SB; Long, MB; White, RR; Sullenger, BA, Crystal structure of an RNA aptamer bound to thrombin., Rna, vol 14 no. 12 (2008), pp. 2504-2512 [10.1261/rna.1239308] [abs].
32. Dollins, CM; Nair, S; Boczkowski, D; Lee, J; Layzer, J M; Gilboa, E; Sullenger, BA, Assembling OX40 aptamers on a molecular scaffold to create a receptor-activating aptamer., Chemistry & Biology, vol 15 no. 7 (2008), pp. 675-682 [10.1016/j.chembio1.2008.05.016] [abs].
33. Biolabs, New England. “In Vitro Digestion of DNA with cas9 Nuclease, S. Pyogenes (M0386).” NEB, https://www.neb.com/protocols/2014/05/01/in-vitro-digestion-of-dna-with-cas9-nuclease-s-pyogenes-m0386.
34. Invitrogen. “Lipofectamine™ CRISPRMAX™ Cas9 Transfection Reagent.” Thermo Fisher Scientific—US, https://www.thermofisher.com/order/catalog/product/CMAX00001.
35. Synthego Performance Analysis, ICE Analysis. 2019. v3.0. Synthego.
36. Addgene.“Lentivirus Production.” Addgene, 26 Aug. 2016, https://www.addgene.org/protocols/lentivirus-production/?gclid=CjOKCQiAoY-PBhCNARIsABcz770ggkRUH.
37. Merten O.-W., Hebben M., Bovolenta C. Production of lentiviral vectors. Mol. Ther.-Methods Clin. Dev. 2016; 3:16017. doi: 10.1038/mtm.2016.17. Transfection Reagent for High-Titer Lentivirus (2007) Clontechniques XXII(4):8
38. Joung, Julia, et al. “Protocol: Genome-Scale CRISPR-Cas9 Knockout and Transcriptional Activation Screening.” Nature Protocols, vol. 12, 2016, pp. 828-863, https://doi.org/10.1101/059626.

1. A method for generating guide nucleic acids that bind a Cas protein, the method comprising:
(a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′portion and an invariant 3′ end,
(b) partitioning candidate guide nucleic acids having an increased binding affinity to the Cas protein from candidate guide nucleic acids having a reduced binding affinity to the Cas protein; and
(c) amplifying the candidate guide nucleic acids having the increased binding affinity to the Cas protein to generate a candidate mixture enriched for candidate guide nucleic acids having binding affinity for the Cas protein.

2. The method of claim 1, wherein the Cas protein is a Cas nickase or catalytically dead Cas (dCas).

3. A method for generating guide nucleic acids that allow cleavage of a double-stranded nucleic acid target when in complex with a Cas protein, the method comprising:
(a) contacting a Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′portion and an invariant 3′ end, thereby forming one or more Cas protein-candidate guide nucleic acid complexes;
(b) partitioning candidate guide nucleic acids having an increased Cas complex cleavage activity by selecting the Cas protein-candidate guide nucleic acid complexes having a free single-stranded DNA 3′ end from candidate guide nucleic acids having a reduced Cas complex cleavage activity; and
(c) amplifying the candidate guide nucleic acids having the increased Cas complex cleavage activity to generate a candidate mixture enriched for candidate guide nucleic acids having Cas complex cleavage activity.

4. The method of claim 3, wherein the Cas protein is further contacted with a polymerase and a labeled nucleotide and the partitioning step comprises labeling the free PAM-distal non-target strand with the labeled nucleotide.

5. The method of claim 4, wherein the polymerase is a terminal deoxynucleotidyl transferase (TdT) and/or the labeled nucleotide is biotin-16-aminoallyl-2′-dATP.

6. (canceled)

7. The method of claim 1, wherein the candidate mixture is enriched for candidate guide nucleic acids having binding affinity for the Cas protein, the method comprising:
(i) contacting the Cas protein with the candidate guide nucleic acids and the target nucleic acid,
(ii) partitioning candidate guide nucleic acids of step (i) having an increased binding affinity to the Cas protein from candidate guide nucleic acids having a reduced binding affinity to the Cas protein; and
(iii) amplifying the candidate guide nucleic acids of step (i) having the increased binding affinity to the Cas protein to generate the candidate mixture enriched for candidate guide nucleic acids having binding affinity for the Cas protein.

8. (canceled)

9. (canceled)

10. The method of claim 1, wherein the Cas9 protein is a Cas9 endonuclease, and the endonuclease is Streptococcus pyogenes Cas9 endonuclease or functional variant thereof or a Staphylococcus aureus Cas9 endonuclease or functional variant thereof and the cleaved double-stranded target nucleic acid further comprises a second label.

11. (canceled)

12. A method for generating a guide nucleic acid having miRNA activity or miRNA modulated activity, the method comprising the methods according to claim 1 and identifying an amplified candidate guide nucleic acid having the miRNA domain, and optionally isolating or purifying the amplified candidate guide nucleic acid having the miRNA domain and wherein the candidate guide nucleic acids comprise a template-conserved miRNA domain.

13. (canceled)

14. (canceled)

15. (canceled)

16. The method of claim 1, wherein the method comprises identifying an amplified candidate guide nucleic acid having Cas complex cleavage activity greater than the template, and optionally isolating or purifying the amplified candidate guide nucleic acid.

17. (canceled)

18. A guide nucleic acid comprising a template-conserved target complementary region and a template-randomized scaffold, wherein the template-conserved target complementary region is configured to hybridize to a double-stranded nucleic acid target proximate to a protospacer adjacent motif (PAM) and wherein the template-randomized region has binding affinity for a Cas protein, wherein the guide nucleic acid comprises any one of the RNAs according to Table 1, Table 2, or Table 3.

19. The guide nucleic acid of claim 18, wherein the guide nucleic acid comprises a functional site, wherein the functional site is optionally a miRNA domain or a miRNA binding domain.

20. (canceled)

21. (canceled)

22. (canceled)

23. The guide nucleic acid of claim 18, wherein the Cas protein the guide nucleic acid binds to is a Cas9 endonuclease, and optionally wherein the Cas9 endonuclease is Streptococcus pyogenes Cas9 endonuclease or Staphylococcus aureus Cas9 endonuclease or functional variants thereof.

24. A mixture comprised of a polymerase, a labeled nucleotide and more than one candidate guide nucleic acid, the candidate guide nucleic acids having a common template-conserved target complementary region and each candidate guide nucleic acid having a distinct template-randomized scaffold, wherein the template-conserved target complementary region is configured to hybridize to a double-stranded DNA proximate to a protospacer adjacent motif (PAM) and wherein the template-randomized scaffold has binding affinity for a Cas protein.

25. (canceled)

26. The mixture of claim 24, wherein the polymerase is a terminal deoxynucleotidyl transferase (TdT) and wherein the labeled nucleotide is biotin-16-aminoallyl-2′-dATP.

27. (canceled)

28. (canceled)

29. The mixture of claim 24, wherein the mixture was made by the method comprising:
(a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′portion and an invariant 3′ end
(b) partitioning candidate guide nucleic acids having an increased binding affinity to the Cas protein from candidate guide nucleic acids having a reduced binding affinity to the Cas protein; and
(c) amplifying the candidate guide nucleic acids having the increased binding affinity to the Cas protein to generate a candidate mixture enriched for candidate guide nucleic acids having binding affinity for the Cas protein.

30. The mixture of claim 24, for use in the method comprising:
(a) contacting the Cas protein with candidate guide nucleic acids and a target nucleic acid, the candidate guide nucleic acids having a template-conserved target complementary region and a template-randomized scaffold, wherein the template-conserved target complementary region is configured to hybridize to a double-stranded DNA proximate to a protospacer adjacent motif (PAM) and wherein the template-randomized scaffold comprises a degenerate nucleic acid 5′portion and an invariant 3′ end,
(b) partitioning candidate guide nucleic acids having an increased binding affinity to the Cas protein from candidate guide nucleic acids having a reduced binding affinity to the Cas protein; and
(c) amplifying the candidate guide nucleic acids having the increased binding affinity to the Cas protein to generate a candidate mixture enriched for candidate guide nucleic acids having binding affinity for the Cas protein.

31. The mixture of claim 24, wherein at least one of the candidate guide nucleic acids is selected from any one of the RNAs according to Table 1, Table 2, or Table 3.

32. A Cas complex comprising:
(a) a Cas protein,
(b) a candidate guide nucleic acid, the candidate guide nucleic acid comprising a template-conserved target complementary region and a template-randomized scaffold having binding affinity for the Cas protein; and
(c) a cleaved target nucleic acid, the cleaved target nucleic acid comprising a free single-stranded labeled 3′ end.

33. (canceled)

34. The Cas complex of claim 32, wherein the Cas protein is a Cas9 endonuclease and wherein the Cas9 endonuclease is Streptococcus pyogenes Cas9 endonuclease, Staphylococcus aureus Cas9 endonuclease or a functional variant thereof and wherein the free single-stranded labeled 3′ end of the target nucleic acid is biotinylated and wherein the cleaved target nucleic acid further comprises a second label.

35. (canceled)

36. (canceled)

37. The Cas complex of claim 32, wherein the candidate guide nucleic comprises one or more candidate guide nucleic acids according to Table 1, Table 2, or Table 3.
